(19) United States
(12) Patent Application Publication
(10) Pub. No.: US 2025/0130225 A1
(43) Pub. Date: Apr. 24, 2025

(54) SUBSTRATES FOR IMAGING GLUCOCEREBROSIDASE ACTIVITY

(71) Applicant: SIMON FRASER UNIVERSITY, Burnaby (CA)

(72) Inventors: David Vocadlo, Burnaby (CA); Matthew Deen, Burnaby (CA); Sha Zhu, Burnaby (CA)

(73) Assignee: SIMON FRASER UNIVERSITY, Burnaby, BC (CA)

(21) Appl. No.: 18/714,811

(22) PCT Filed: Dec. 3, 2022
(86) PCT No.: PCT/IB2022/061738

§ 371 Date: May 30, 2024

Related U.S. Application Data
(60) Provisional application No. 63/285,996, filed on Dec. 3, 2021.

(51) Int. Cl. G01N 33/542; C07H 15/222; C07H 23/00; G01N 21/64; G01N 33/573

(52) U.S. Cl. CPC G01N 33/542; C07H 15/222; C07H 23/00; G01N 21/6428; G01N 33/573; G01N 2021/6432; G01N 2333/924

(57) ABSTRACT

The application relates to fluorescence-quenched substrates of Formula (I) for an acid β-glucosidase (GCase) and their use in a method for determining GCase activity in a cell or tissue, a method for localizing GCase activity within a cell, a method for determining the effect of a GCase modulator in a cell, a method for determining the efficacy if a GCase-directed therapy, and a method of screening for a GCase activity enhancer.

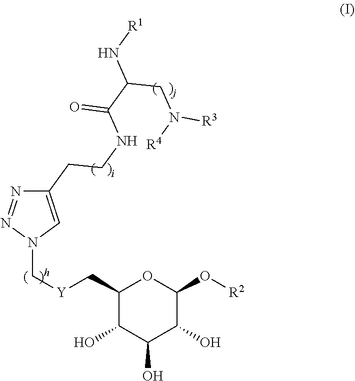

### FIELD OF THE INVENTION

The application relates to fluorescence-quenched glucocerebrosidase substrates and uses thereof.

### BACKGROUND OF THE INVENTION

Acid β-glucosidase (GCase, EC 3.2.1.45) is a membrane associated glycoprotein that cleaves glucosylceramide (GlcCer) into glucose and ceramide, and is a lysosomal lipid hydrolase that is a member of CAZy glycoside hydrolase family GH30.¹ Homozygous loss-of-function mutations^{2,3} in the gene encoding GCase (GBA1) cause Gaucher disease (GD), a lysosomal storage disorder with a recessive pattern of inheritance. There are greater than 280 known mutations,⁴ most of which lead to single amino acid changes and the resulting mutant enzymes typically have some residual activity. Decreased GCase activity in humans results in the detrimental accumulation of glucosylceramide within the lysosomes of affected tissues, which is a key pathological feature of GD. GD has been classified into three types;⁵ non-neuronopathic (type 1 GD) is the most common of these types, affecting 1 in 10,000 within the general population. Neuronopathic GD includes both types 2 and 3, which are fatal. Type 2 is more severe than type 3, exhibiting central nervous system (CNS) involvement that manifests within a year of birth. Type 3 GD is milder and manifests later in life but still usually leads to death by the age of 30.

Two different types of treatments have been introduced for type 1 GD. One is enzyme replacement therapy (ERT),⁶ which is based on chronic intravenous administration of recombinant wild type GCase. The other is substrate reduction therapy (SRT),^{6,7} which is based on chronic oral administration of an inhibitor of glucosylceramide synthase, which is the enzyme catalyzing the formation of glucosylceramide. Over the past decade, an alternative experimental approach called chaperone therapy has gained attention.⁸⁻¹¹

Mutations in GBA1 (also sometimes defined as GBA) have also been implicated in Lewy body diseases (LBDs) such as Parkinson's disease (PD),¹²⁻¹⁴ emerging as a major genetic risk factor for PD¹⁵⁻¹⁷ and exacerbating the progression of disease.¹⁸⁻²¹ Furthermore, PD patients, regardless of whether they carry mutations in GCase, appear to have lower GCase activity in the central nervous system.^{18,22} Transgenic overexpression of GCase in a PD mouse model reduced Lewy body pathology and has suggested that loss of function of GCase contributes to α-synuclein toxicity.^{23,24}

Most mutations in the GBA1 gene lead to expression of unstable mutant forms of GCase, which do not reach the lysosome and instead undergo endoplasmic reticulum associated degradation (ERAD). As a result, levels of active GCase within lysosomes are diminished in those individuals carrying such mutant GBA1 alleles. However, small molecule ligands of GCase that are inhibitors can stabilize mutant GCase, serving as chaperones and helping the mutant enzyme reach lysosomes. After diffusion of the small molecule inhibitor out of the cell, mutant GCase within lysosomes is stable and functional. A benefit of this approach is that, unlike recombinant enzyme delivered intravenously, such small molecule inhibitors are generally brain penetrant.

Generally, the efficiency of inhibitory chaperones for GCase is established by monitoring increases in the levels of this enzyme by immunoblotting of cell lysates,²⁵ or measuring the total activity of GCase in cell lysates using in vitro enzyme assays.²⁵⁻²⁷ The same is true for approaches including enzyme replacement and gene therapy.^{28,29} There are however few methods to visualize and quantify the effects of pharmacological chaperones directly in cells.

Activity-based probes (ABPs) have been used to monitor total levels of active GCase enzyme in cell lysates and image functional GCase within fixed cells.³⁰ One limitation to using this approach to monitor GCase activity, however, is that these probes lead to complete inactivation of GCase over time, even in the presence of enzyme modulators including competitive inhibitors.

Development of fluorescence-quenched substrates to measure the activity of glycoside hydrolases has seen slow growth and has provided limited utility for monitoring the endogenous activity of glycoside hydrolases in mammalian cells or tissue. Fluorogenic substrates to monitor abundantly-expressed marker glycosidases, such as Escherichia coli and senescence-associated β-galactosidase, have been described.³¹⁻³³ However, signal intensity and assay to signal intensity is still limited. Thus, initial efforts have yielded probes that also covalently label, and in turn inactivate, the target enzyme by the formation of a transient reactive quinine methide intermediate within the enzyme active site.³⁴ For example, fluorogenic substrates for GCase have been described and used to quantify GCase activity but have not proven useful for direct, accurate, and selective monitoring of active enzyme by live cell imaging.³⁵ A compound that uses Forster resonance energy transfer was reported to be able to measure GCase activity in live cells³⁵.

### SUMMARY OF THE INVENTION

The present invention provides, in part, fluorescence-quenched substrate compounds for GCase, uses of the compounds, methods for monitoring GCase activity within cells or tissue, methods for localization of GCase activity within lysosomes, methods for monitoring the effects of a GCase inhibitor in cells or tissue, methods for monitoring the effects of a GCase chaperone in cells or tissue, methods for monitoring the effects of a GCase activator in cells or tissue, methods for monitoring the effects of enzyme replacement or augmentation using exogenous GCase in cells or tissue, methods for monitoring the effects of gene therapy approaches to augmenting GCase activity within cells or tissue, methods for detecting increased or decreased levels of GCase that may be associated with disease, including for example, Parkinson's disease, methods for monitoring GCase activity within cells or tissue as a biomarker for a GCase-directed therapy, or methods for conducting a cell-based library screen to identify a GCase activity enhancer. In alternative aspects, such compounds may be useful in identifying GCase chaperones studying the trafficking and regulation of GCase within cells, screening for endogenous protein modifiers of GCase activity, as well as screening for activators that function within cells to influence GCase activity.

In one aspect, the invention provides a compound of Formula (I), or an acceptable salt thereof:

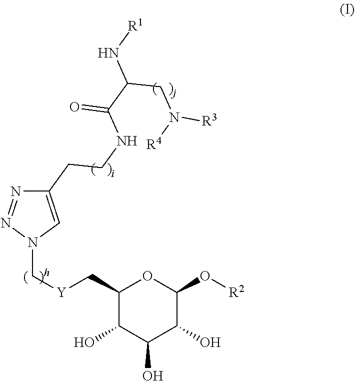

where
R¹ may be a suitable fluorophore or a suitable quencher;
R² may be

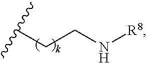

where k may be an integer from 1 to 10, and when R¹ is a suitable fluorophore, R⁸ may be a suitable quencher; or when R¹ is a suitable quencher, R⁸ may be a suitable fluorophore, or
R² may be

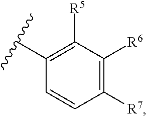

where R⁵, R⁶ and R⁷ may each independently be H, F, NO₂, or a suitable quencher;
R³ and R⁴ may each independently be H, an alkyl chain of one to 4 carbons, or a peptide of between 1 and 10 amino acids in length that is covalently linked using an amide bond, or another suitable bioconjugation reaction, or using a suitable click reaction;
h may be an integer from 1 to 5;
i may be an integer from 1 to 5;
j may be a n integer from 1 to 5; and
Y may be O, S, NH or CH₂.

In some embodiments, the invention provides a compound of Formula (II), or an acceptable salt thereof:

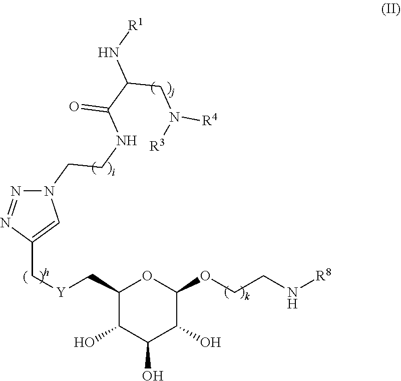

where R¹ may be a suitable fluorophore and R⁸ may be a suitable quencher; or R¹ may be a suitable quencher and R⁸ may be a suitable fluorophore; R³ and R⁴ may each independently be hydrogen (H), an alkyl chain of one to 4 carbons, or a peptide of between 1 and 10 amino acids in length that is covalently linked using an amide bond, or another suitable bioconjugation reaction, or using a suitable click reaction; h may be an integer from 1 to 5; i may be an integer from 1 to 5; j may be a n integer from 1 to 5; k may be an integer from 1 to 10; Y may be an O or S or NH or CH₂.

In some embodiments, the invention provides a compound of Formula (IIa), or an acceptable salt thereof:

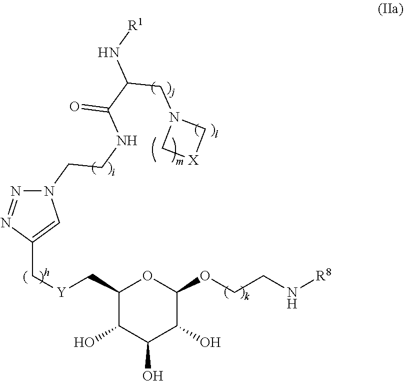

where R¹ may be a suitable fluorophore and R⁸ may be a suitable quencher; or R¹ may be a suitable quencher and R⁸ may be a suitable fluorophore; h may be an integer from 1 to 5; i may be an integer from 1 to 5; j may be a n integer from 1 to 5; k may be an integer from 1 to 10; m may be an integer from 1 to 4; 1 may be an integer from 1 to 4; and X may be O or S or NH or CH₂ or NR⁹ where R⁹ may be a short alkyl chain of 1 to 4 carbon atoms; and Y may be O or S or NH or CH₂.

In some embodiments, the invention provides a compound of Formula (IIb), or an acceptable salt thereof:

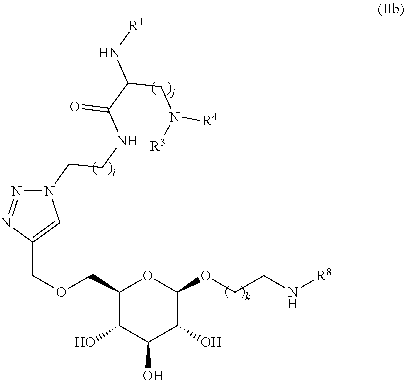

where R¹ may be a suitable fluorophore and R⁸ may be a suitable quencher; or R¹ may be a suitable quencher and R⁸ may be a suitable fluorophore; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; i may be an integer from 1 to 5; j may be an integer from 1 to 5; k may be an integer from 1 to 10.

In some embodiments, the invention provides a compound of Formula (IIc), or an acceptable salt thereof

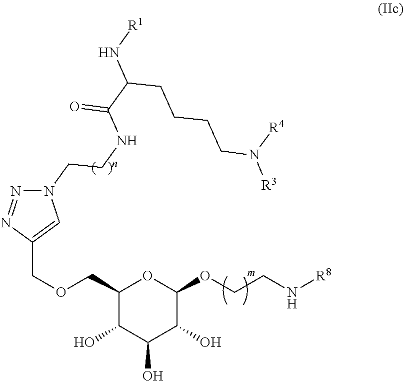

where R¹ may be a suitable fluorophore and R⁸ may be a suitable quencher; or R¹ may be a suitable quencher and R² may be a suitable fluorophore; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; n may be an integer from 1 to 10; and m may be an integer from 1 to 5.

In some embodiments, the invention provides a compound of Formula (IId), or an acceptable salt thereof.

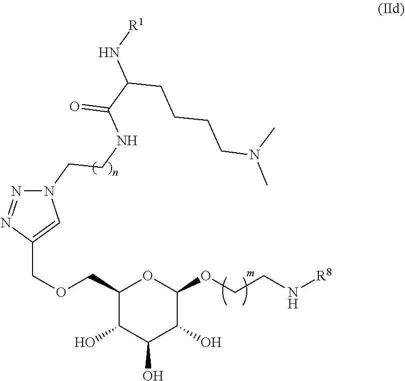

where R¹ may be a suitable fluorophore and R⁸ may be a suitable quencher; or R¹ may be a suitable quencher and R⁸ may be a suitable fluorophore; n may be an integer from 1 to 10; and m may be an integer from 1 to 5.

In some embodiments, the invention provides a compound of Formula (IIe), or an acceptable salt thereof

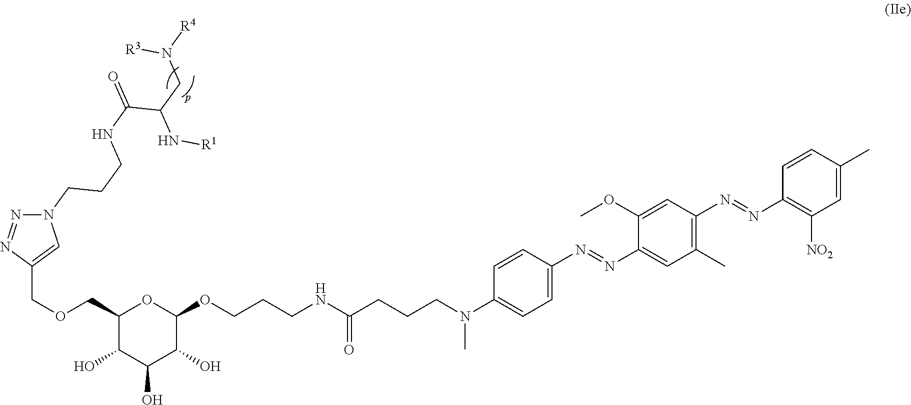

where R¹ may be a suitable fluorophore; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; n may be an integer from 1 to 10; and m may be an integer from 1 to 5; p may be an integer from 01 to 5.

In some embodiments, the invention provides a compound of Formula (IIf), or an acceptable salt thereof.

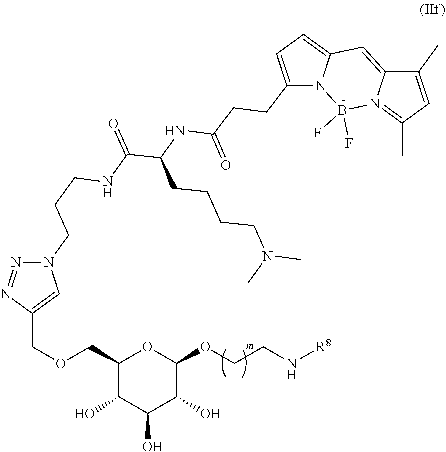

where R⁸ may be a suitable quencher; m may be an integer from 1 to 5.

In some embodiments, the invention provides a compound of Formula (IIg), or an acceptable salt thereof:

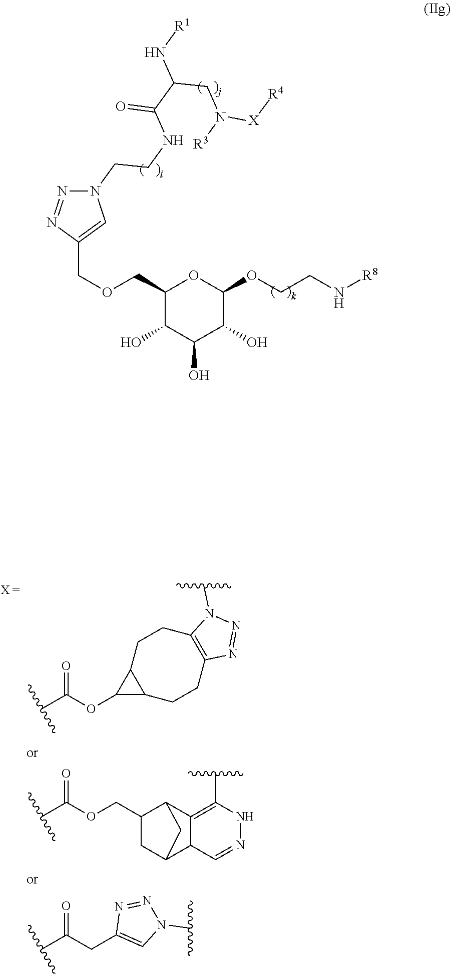

where R¹ may be a suitable fluorophore and R⁸ may be a suitable quencher; or R¹ may be a suitable quencher and R⁸ may be a suitable fluorophore; R³ may selected from a list of hydrogen, an alkyl chain of one to 4 carbons; and R⁴ may be a peptide between 1 and 10 amino acid residues in length; X may be the product of a click reaction, for example, as indicated herein; i may be an integer from 1 to 5; j may be a n integer from 1 to 5; k may be an integer from 1 to 10.

In some embodiments, the invention provides a compound of Formula (IIh), or an acceptable salt thereof:

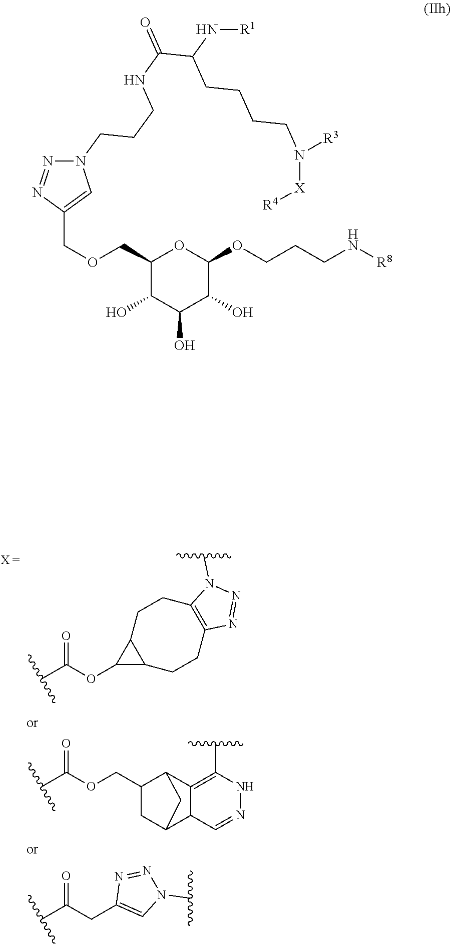

where R¹ may be a suitable fluorophore and R⁸ may be a suitable quencher; or R¹ may be a suitable quencher and R⁸ may be a suitable fluorophore; R³ may selected from a list of hydrogen, an alkyl chain of one to 4 carbons; R⁴ may be a peptide between 1 and 10 amino acid residues in length; X may be the product of a click reaction, for example, as indicated herein.

In some embodiments, the invention provides a compound of Formula (IIi), or an acceptable salt thereof:

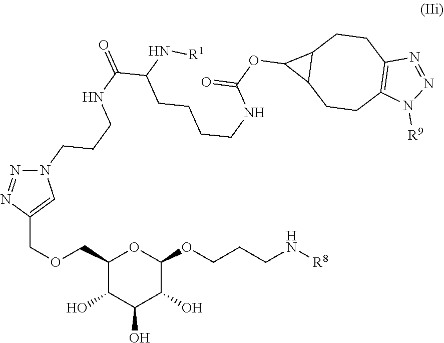

where R¹ may be a suitable fluorophore and R⁸ may be a suitable quencher; or R¹ may be a suitable quencher and R⁸ may be a suitable fluorophore; R⁹ may be an oligopeptide between 1 and 10 amino acid residues in length.

In some embodiments, the invention provides a compound of Formula (IIj), or an acceptable salt thereof:

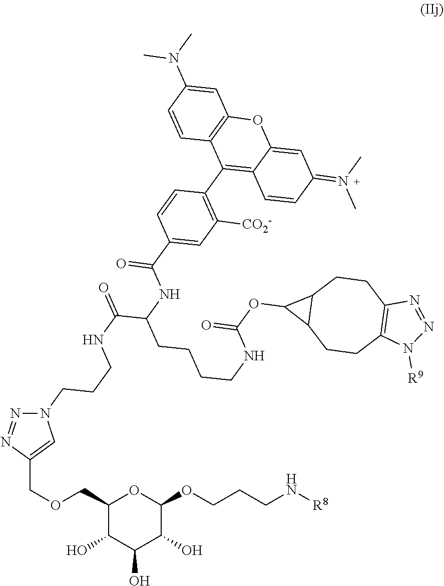

where R² may be a suitable quencher; R⁹ may be an oligopeptide between 1 and 10 amino acids in length.

In some embodiments, the invention provides a compound of Formula (IIk), or an acceptable salt thereof:

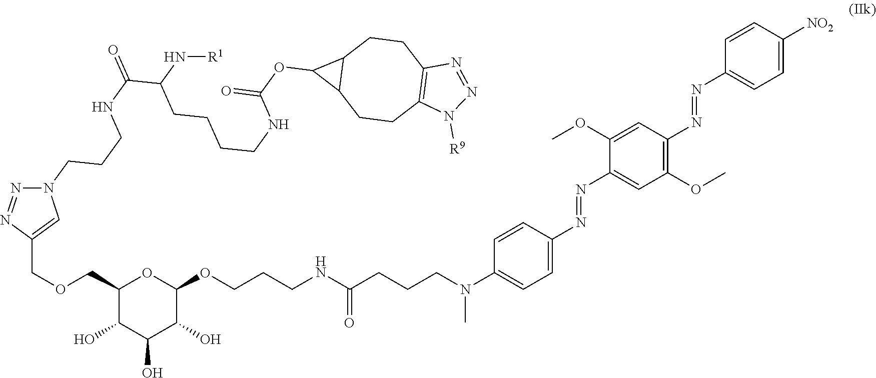

where R¹ may be a suitable fluorophore; R⁹ may be an oligopeptide between 1 and 10 amino acid residues in length.

In some embodiments, the invention provides a compound of Formula (IIl), or an acceptable salt thereof.

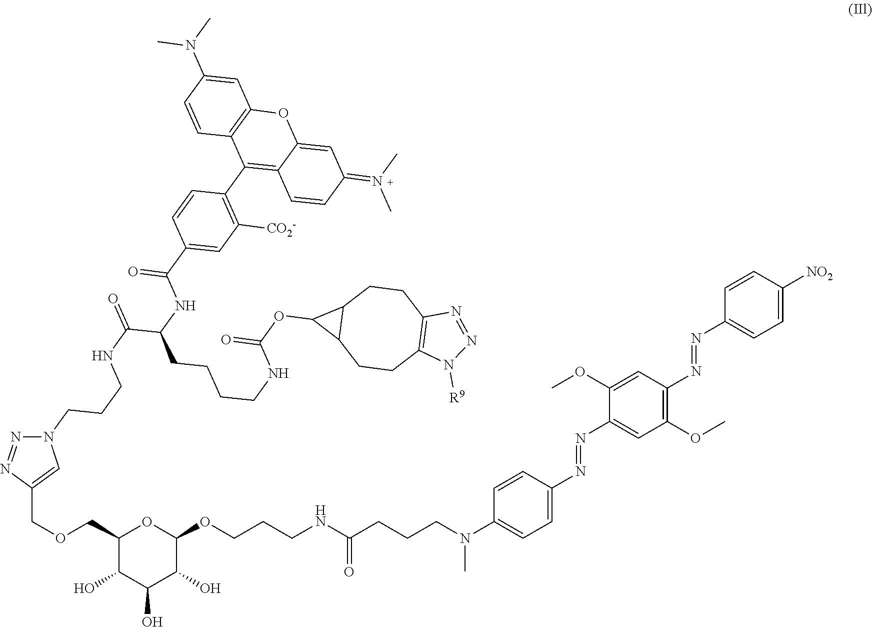

where R⁹ may be an oligopeptide between 1 and 10 amino acid residues in length.

In some embodiments, the invention provides a compound of Formula (III), or an acceptable salt thereof.

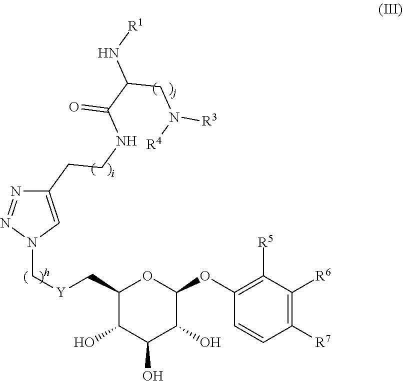

where R¹ may be a suitable fluorophore; R³ and R⁴ may each independently be H, an alkyl chain of one to 4 carbons or a peptide of between 1 and 10 amino acids in length that is covalently linked using an amide bond, or another suitable bioconjugation reaction, or using a suitable click reaction; R⁵, R⁶ and R⁷ may each independently be H, F, NO₂, or a suitable quencher; h may be an integer from 1 to 5; i may be an integer from 1 to 5; j may be a n integer from 1 to 5; Y may be O, S, NH or CH₂.

In some embodiments, the invention provides a compound of Formula (IIIa), or an acceptable salt thereof.

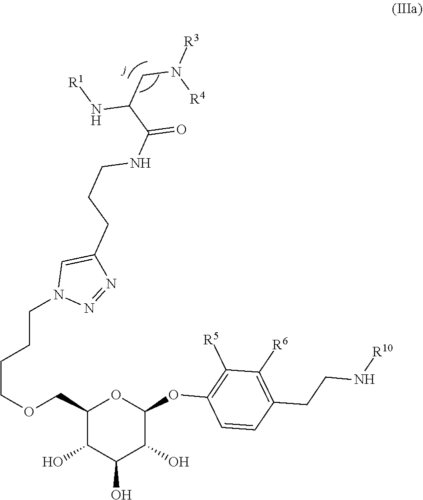

where R¹ may be a suitable fluorophore; R³ and R⁴ may each independently be H, an alkyl chain of one to 4 carbons or a peptide of between 1 and 10 amino acids in length that is covalently linked using an amide bond, or another suitable bioconjugation reaction, or using a suitable click reaction; R⁵ and R⁶ may each independently be H, F, or NO₂; R¹⁰ may be a suitable quencher; j may be a n integer from 1 to 5.

In some embodiments, the invention provides a compound of Formula (IIIb), or an acceptable salt thereof.

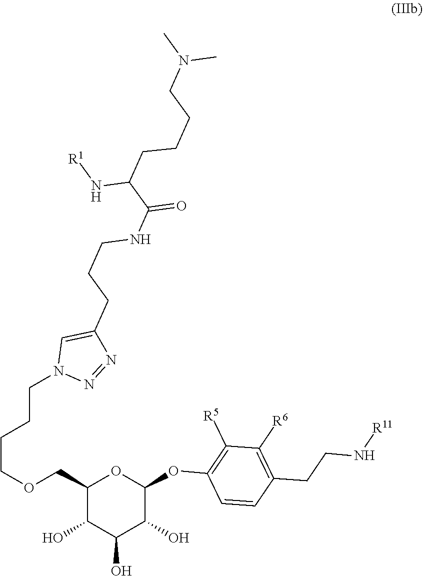

where R¹ may be a suitable fluorophore; R⁵ and R⁶ may each independently H, F, or NO₂; R¹¹ may be a suitable quencher.

In some embodiments, the invention provides a compound of Formula (IIIc), or an acceptable salt thereof.

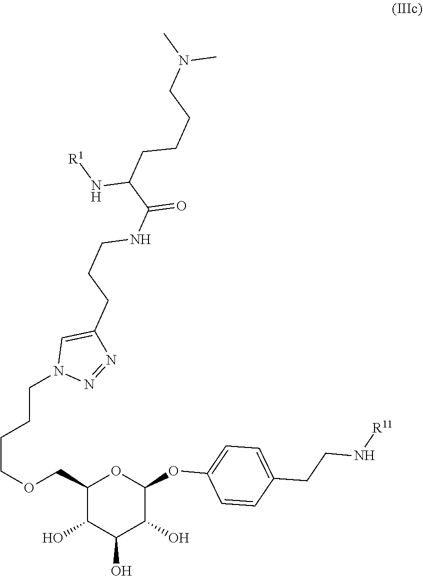

where R¹ may be a suitable fluorophore and R¹¹ may be a suitable quencher.

In some embodiments, the invention provides a compound of Formula (IIId), or an acceptable salt thereof.

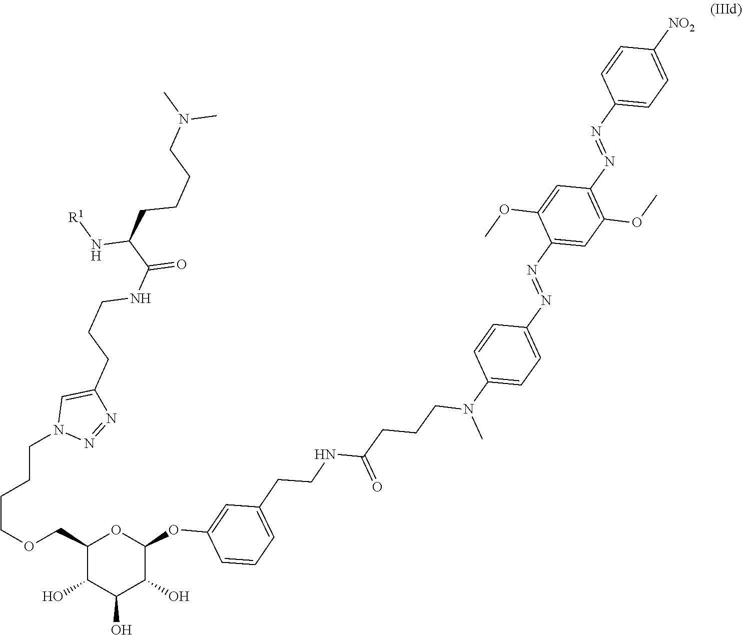

where R¹ may be a suitable fluorophore.

In some embodiments, the invention provides a compound of Formula (IIIe), or an acceptable salt thereof

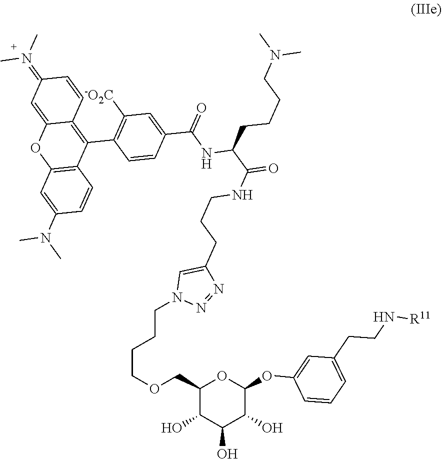

where R¹¹ may be a suitable quencher.

In alternative aspects, the invention provides a method for determining GCase activity within lysosomes of a cell, the method comprising: (i) providing a test cell and a control cell; (ii) contacting the test cell with a compound as described herein, for example, a compound of Formula (I), (II), (IIa)-(IIl), (III), (IIIa)-(IIIe), or an acceptable salt thereof and (iii) determining fluorescence intensity in the test cell and the control cell, wherein an increase in fluorescence intensity of the test cell when compared to the control cell is indicative of GCase activity. The cells may be derived from a tissue. The tissue may be a skin punch or blood.

In alternative aspects, the invention provides a method for localizing GCase activity within a cell, the method comprising: (i) providing a test cell and a control cell; (ii) contacting the test cell with a compound as described herein, for example, a compound of Formula (I), (II), (IIa)-(IIl), (III), (IIIa)-(IIIe), or an acceptable salt thereof, (iii) visualizing fluorescence intensity in the test cell and the control cell, wherein an increase in fluorescence intensity in a location in the test cell when compared to the fluorescence intensity in a corresponding location in the control cell is indicative of GCase activity. The location may be the endoplasmic reticulum, Golgi apparatus or lysosomal compartment. The cells may be derived from a tissue. The tissue may be a skin punch or blood.

In alternative aspects, the invention provides a method for determining the effect of a GCase modulator of GCase activity within lysosomes of a cell, the method comprising: (i) providing a test cell and a control cell; (ii) contacting the test cell with a GCase modulator; (iii) contacting the test cell and the control cell with a compound as described herein, for example, a compound of Formula (I), (II), (IIa)-(IIl), (III), (IIIa)-(IIIe), or an acceptable salt thereof, and (iv) determining fluorescence intensity in the test cell and the control cell, wherein a difference in fluorescence intensity of the test cell when compared to the control cell is indicative of GCase modulation. The GCase modulator may be a GCase inhibitor, or a GCase activator, or a GCase chaperone. The cells may be derived from a tissue. The tissue may be a skin punch or blood.

In alternative aspects, the invention provides a method for determining the efficacy of a GCase-directed therapy, the method comprising: (i) providing a test cell, wherein the test cell is obtained from a subject treated with a GCase-directed therapy, and a control cell, wherein the control cell is obtained from a subject not treated with a GCase-directed therapy; (ii) contacting the test cell and the control cell with a compound as described herein, for example, a compound of Formula (I), (II), (IIa)-(IIl), (III), (IIIa)-(IIIe), or an acceptable salt thereof, and (iii) measuring fluorescence intensity in the test cell and the control cell, wherein a difference in fluorescence intensity of the test cell when compared to the control cell is representative of the efficacy of the GCase-directed therapy. The GCase directed therapy may be an enzyme replacement or augmentation therapy, a gene therapy, or a cell therapy that is expected to lead to increased levels of GCase in cells. The cells may be derived from a tissue. The tissue may be a skin punch or blood.

In alternative aspects, the invention provides a method for screening for a GCase activity enhancer, the method comprising: (i) providing a test cell and a control cell; (ii) contacting the test cell with a test compound; (iii) contacting the test cell and the control cell with a compound as described herein, for example, a compound of Formula (I), (II), (IIa)-(IIl), (III), (IIIa)-(IIIe), or an acceptable salt thereof, and (iv) determining fluorescence intensity in the test cell and the control cell, wherein an increase in fluorescence intensity in the test cell when compared to the control cell indicates that the test compound is a GCase activity enhancer.

In alternative aspects, the invention provides a method for determining GCase activity within lysosomes of a cell, the method comprising: (i) providing a test cell and a control cell; (ii) contacting the test cell with a compound of Formula (I), (II), (IIa)-(IIl), (III), (IIIa)-(IIIe), or an acceptable salt thereof, and (iii) determining fluorescence intensity in the test cell and the control cell, wherein an increase in fluorescence intensity of the test cell when compared to the control cell is indicative of GCase activity. In this aspect, when the compound is for example the compound of Formula (II), the difference in fluorescence intensity between the test cell and the control cell, due to either the compound of Formula (IV) or the compound of Formula (V) in the test cell, is indicative of GCase activity. The cells may be derived from a tissue. The tissue may be a skin punch or blood.

In alternative aspects, the invention provides a method for localizing GCase activity within a cell, the method comprising: (i) providing a test cell and a control cell; (ii) contacting the test cell with a compound of Formula (I), (II), (IIa)-(IIl), (III), (IIIa)-(IIIe), or an acceptable salt thereof; and (iii) visualizing fluorescence intensity in the test cell and the control cell, wherein an increase in fluorescence intensity in a location in the test cell when compared to the fluorescence intensity in a corresponding location in the control cell is indicative of GCase activity. In this aspect, when the compound is for example the compound of Formula (II), the increased fluorescence intensity in a location in the test cell when compared to the fluorescence intensity in a corresponding location in the control cell, due to either the compound of Formula (IV) or the compound of Formula (V) in the test cell, is indicative of GCase activity. The location may be the endoplasmic reticulum, Golgi apparatus or lysosomal compartment. The cells may be derived from a tissue. The tissue may be a skin punch or blood.

In alternative aspects, the invention provides a method for determining the effect of a GCase modulator of GCase activity within lysosomes of a cell, the method comprising: (i) providing a test cell and a control cell; (ii) contacting the test cell with a GCase modulator; (iii) contacting the test cell and the control cell with a compound of Formula (I), (II), (IIa)-(IIl), (III), (IIIa)-(IIIe), or an acceptable salt thereof; and (iv) determining fluorescence intensity in the test cell and the control cell, wherein a difference in fluorescence intensity of the test cell when compared to the control cell is indicative of GCase modulation. In this aspect, when the compound is for example the compound of Formula (II), the difference in fluorescence intensity between the test cell and the control cell, due to a difference in concentration of either the compound of Formula (IV) or the compound of Formula (V) between the test cell and the control cell, is indicative of GCase modulation. The GCase modulator may be a GCase inhibitor, or a GCase activator, or a GCase chaperone. The cells may be derived from a tissue. The tissue may be a skin punch or blood.

In alternative aspects, the invention provides a method for determining the efficacy of a GCase-directed therapy, the method comprising: (i) providing a test cell, wherein the test cell is obtained from a subject treated with a GCase-directed therapy, and a control cell, wherein the control cell is obtained from a subject not treated with a GCase-directed therapy; (ii) contacting the test cell and the control cell with a compound of Formula (I), (II), (IIa)-(IIl), (III), (IIIa)-(IIIe), or an acceptable salt thereof, and (iii) measuring fluorescence intensity in the test cell and the control cell, wherein a difference in fluorescence intensity of the test cell when compared to the control cell is representative of the efficacy of the GCase-directed therapy. In this aspect, when the compound is for example the compound of Formula (II), the difference in fluorescence intensity between the test cell and the control cell, due to a difference in concentration of either the compound of Formula (IV) or the compound of Formula (V) between the test cell and the control cell, is representative of the efficacy of the GCase-directed therapy. The GCase directed therapy may be an enzyme replacement or augmentation therapy, a gene therapy, or a cell therapy that is expected to lead to increased levels of GCase in cells. The cells may be derived from a tissue. The tissue may be a skin punch or blood.

In alternative aspects, the invention provides a method for screening for a GCase activity enhancer, the method comprising: (i) providing a test cell and a control cell; (ii) contacting the test cell with a test compound; (iii) contacting the test cell and the control cell with a compound of Formula (I), (II), (IIa)-(IIl), (III), (IIIa)-(IIIe), or an acceptable salt thereof; and (iv) determining fluorescence intensity in the test cell and the control cell, wherein an increase in fluorescence intensity in the test cell when compared to the control cell indicates that the test compound is a GCase activity enhancer. In this aspect, when the compound is for example the compound of Formula (II), an increase in fluorescence intensity in the test cell compared to the control cell, due to an increase in concentration of either the compound of Formula (IV) or the compound of Formula (V) in the test cell compared to the control cell, indicates that the test compound is a GCase activity enhancer.

In alternative aspects, the invention provides a method for determining GCase activity within lysosomes of a cell, the method comprising: (i) providing a test cell and a control cell; (ii) contacting the test cell with a compound of Formula (I), (II), (IIa)-(IIl), (III), (IIIa)-(IIIe), or an acceptable salt thereof; and (iii) determining fluorescence intensity in the test cell and the control cell, wherein an increase in fluorescence intensity of the test cell when compared to the control cell is indicative of GCase activity. In this aspect, when the compound is for example the compound of Formula (III), the difference in fluorescence intensity between the test cell and the control cell, due to either the compound of Formula (VI) or the compound of Formula (VII) in the test cell, is indicative of GCase activity. The cells may be derived from a tissue. The tissue may be a skin punch or blood.

In alternative aspects, the invention provides a method for localizing GCase activity within a cell, the method comprising: (i) providing a test cell and a control cell; (ii) contacting the test cell with a compound of Formula (I), (II), IIa)-(IIl), (III), (IIIa)-(IIIe), or an acceptable salt thereof; and (iii) visualizing fluorescence intensity in the test cell and the control cell, wherein an increase in fluorescence intensity in a location in the test cell when compared to the fluorescence intensity in a corresponding location in the control cell is indicative of GCase activity. In this aspect, when the compound is for example the compound of Formula (III), the increased fluorescence intensity in a location in the test cell when compared to the fluorescence intensity in a corresponding location in the control cell, due to either the compound of Formula (VI) or the compound of Formula (VII) in the test cell, is indicative of GCase activity. The location may be the endoplasmic reticulum, Golgi apparatus or lysosomal compartment. The cells may be derived from a tissue. The tissue may be a skin punch or blood.

In alternative aspects, the invention provides a method for determining the effect of a GCase modulator of GCase activity within lysosomes of a cell, the method comprising: (i) providing a test cell and a control cell; (ii) contacting the test cell with a GCase modulator; (iii) contacting the test cell and the control cell with a compound of Formula (I), (II), IIa)-(IIl), (III), (IIIa)-(IIIe), or an acceptable salt thereof; and (iv) determining fluorescence intensity in the test cell and the control cell, wherein a difference in fluorescence intensity of the test cell when compared to the control cell is indicative of GCase modulation. In this aspect, when the compound is for example the compound of Formula (III), the difference in fluorescence intensity between the test cell and the control cell, due to a difference in concentration of either the compound of Formula (VI) or the compound of Formula (VII) between the test cell and the control cell, is indicative of GCase modulation. The GCase modulator may be a GCase inhibitor, or a GCase activator, or a GCase chaperone. The cells may be derived from a tissue. The tissue may be a skin punch or blood.

In alternative aspects, the invention provides a method for determining the efficacy of a GCase-directed therapy, the method comprising: (i) providing a test cell, wherein the test cell is obtained from a subject treated with a GCase-directed therapy, and a control cell, wherein the control cell is obtained from a subject not treated with a GCase-directed therapy; (ii) contacting the test cell and the control cell with a compound of Formula (I), (II), IIa)-(IIl), (III), (IIIa)-(IIIe), or an acceptable salt thereof and (iii) measuring fluorescence intensity in the test cell and the control cell, wherein a difference in fluorescence intensity of the test cell when compared to the control cell is representative of the efficacy of the GCase-directed therapy. In this aspect, when the compound is for example the compound of Formula (III), the difference in fluorescence intensity between the test cell and the control cell, due to a difference in concentration of either the compound of Formula (VI) or the compound of Formula (VII) between the test cell and the control cell, is representative of the efficacy of the GCase-directed therapy. The GCase directed therapy may be an enzyme replacement or augmentation therapy, a gene therapy, or a cell therapy that is expected to lead to increased levels of GCase in cells. The cells may be derived from a tissue. The tissue may be a skin punch or blood.

In alternative aspects, the invention provides a method for screening for a GCase activity enhancer, the method comprising: (i) providing a test cell and a control cell; (ii) contacting the test cell with a test compound; (iii) contacting the test cell and the control cell with a compound of Formula (I), (II), IIa)-(IIl), (III), (IIIa)-(IIIe), or an acceptable salt thereof; and (iv) determining fluorescence intensity in the test cell and the control cell, wherein an increase in fluorescence intensity in the test cell when compared to the control cell indicates that the test compound is a GCase activity enhancer. In this aspect, when the compound is for example the compound of Formula (III), an increase in fluorescence intensity in the test cell compared to the control cell, due to an increase in concentration of either the compound of Formula (VI) or the compound of Formula (VII) in the test cell compared to the control cell, indicates that the test compound is a GCase activity enhancer.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 is a graph showing the fluorescence spectrum of substrate 1 (also referred to as MDFL1-dML).

FIG. 2 is a graph showing the determination of quenching efficiency of substrate 1 (also referred to as MDFL1-dML).

FIG. 3 is a graph showing the in vitro GCase assay of substrate 1 (also referred to as MDFL1-dML).

FIG. 4 is a graph showing that GCase fluorescence quenched substrate 1 (also referred to as MDFL1-dML) shows time dependent increases in fluorescence upon incubation with SK-N-SH cells. From the images, the total mean integrated intensity was measured for the FITC channel and divided by the number of cells within the imaging site as determined by the DAPI staining. Measurements at various time points during incubation of various concentrations of substrate 1 (also referred to as MDFL1-dML) reveals a linear increase from 30 minutes to 3 hours. Error bars represent the standard deviation obtained from triplicates (3 separate wells, 9 sites imaged in each well).

FIG. 5 is a graph showing that GCase fluorescence quenched substrate 1 (also referred to as MDFL1-dML) shows dose dependent increase in fluorescence upon incubation with SK-N-SH cells. Fluorescence intensity per cell at various concentrations of substrate 1 (also referred to as MDFL1-dML) reveals a linear increase in fluorescence as a function of substrate concentration. Error bars represent the standard deviation obtained from triplicates (3 separate wells, 9 sites imaged in each well).

FIGS. 6A-B show that processing of fluorescence quenched substrate 1 (also referred to as MDFL1-dML) is GCase dependent. Treatment of SK-N-SH cells with the selective GCase inhibitor AT3375 leads to decreased fluorescence associated with processing of fluorescence quenched substrate 1 (also referred to as MDFL1-dML) suggesting specific turnover. (A) Fluorescence images obtained in the absence of AT3375 and presence of AT3375; (B) Quantitative relative fluorescence intensity per cell for cells treated with substrate 1 (also referred to as MDFL1-dML) and substrate 1 (also referred to as MDFL1-dML) plus AT3375. Error bars represent the standard deviation obtained from triplicates (3 separate wells, 9 sites imaged in each well). The white scale bars represent 100 μm.

FIGS. 7A-B show that processing of fluorescence quenched substrate 1 occurs in lysosomes. Fluorescence was measured for the FITC channel (BODIPY® Fl fluorescence) and the TRITC channel (LysoTracker® Red DND-99). (A) The fluorescent signal from fluorescence quenched substrate 1 (also referred to as MDFL1-dML) is co-localized with LysoTracker® Red. The merged image channels show significant co-localization between signal in the FITC (substrate 1 (also referred to as MDFL1-dML)) and TRITC (LysoTracker® Red) channels with a (B) Pearson's correlation coefficient value of 0.72 suggesting substrate 1 (also referred to as MDFL1-dML) is turned over by GCase localized within lysosomes. Representative images of the 9 sites imaged for each well in a 96-well plate. The white scale bars represent 100 μm.

FIG. 8 is a graph showing the determination of the IC₅₀ value of the selective GCase inhibitor AT3375 in SK-N-SH cells using fluorescence quenched substrate 1 (also referred to as MDFL1-dML).

FIGS. 9A-B show the improved signal intensity of fluorescence quenched substrate 1 (also referred to as MDFL1-dML) over fluorescence quenched substrate 9b. (A) The fluorescence images were obtained by treatment of SK-N-SH cells with substrate 9b (10 μM) in the presence or absence of AT3375 or by treatment of SK-N-SH cells with substrate 1 (10 μM) (also referred to as MDFL1-dML) in the presence or absence of AT3375. (B) Quantitative relative fluorescence intensity per cell for cells treated with substrate 1 (also referred to as MDFL1-dML) and substrate 1 (also referred to as MDFL1-dML) plus AT3375 and substrate 9b and substrate 9b plus AT3375. Error bars represent the standard deviation obtained from triplicates (3 separate wells, 9 sites imaged in each well). The white scale bars represent 100 μm.

FIGS. 10A-B show the improved cellular retention of fluorescence quenched substrate 1 (also referred to as MDFL1-dML) over fluorescence quenched substrate 9b. (A) Quantitative relative fluorescence intensity per cell for cells treated with substrate 9b which after a 2-hour incubation were washed and read in a media which contained a high concentration of AT3375 (10 μM) to stop hydrolysis by GCase. The cells were then imaged at time points from 0 minutes to 3 hours. (B) Quantitative relative fluorescence intensity per cell for cells treated with substrate 1 (also referred to as MDFL1-dML) which after a 2-hour incubation were washed and read in media which contained a high concentration of AT3375 (10 μM) to stop hydrolysis of substrate 1 (also referred to as MDFL1-dML). The cells were then imaged at time points from 0 minutes to 3 hours.

FIG. 11 is a graph showing a comparison of Michaelis-Menten plots of hydrolysis of fluorescence quenched substrate 3 as compared to fluorescence quenched substrate 9b by GCase.

FIG. 12 is a graph showing that the fluorescence quenched substrate 4 has improved signal intensity as compared to fluorescence quenched substrate 9b.

FIG. 13 is a graph showing that fluorescence quenched substrate 4 shows time dependent increase in fixed SK-N-SH cells. Error bars represent the standard deviation obtained from triplicates (3 separate wells, 9 sites imaged in each well).

FIG. 14 shows processing of fluorescence quenched substrate 4 occurs in lysosomes as determined by co-localization with immunocytochemistry-stained LAMP-2 positive vesicles. Fluorescence is measured for the TRITC channel (TAMRA fluorescence) and the FITC channel (LAMP-2 antibody). The merged image of the FITC and TRITC channels show significant co-localization with a Pearson's correlation coefficient value of 0.55, suggesting substrate 1 (also referred to as MDFL1-dML) is turned over by GCase localized within lysosomes. Error bars represent the standard deviation obtained from triplicates (3 separate wells, 9 sites imaged in each well).

FIGS. 15A-B show that the signal from fluorescence quenched substrate 4 in fixed SK-N-SH cells is stable 6 days after paraformaldehyde treatment. (A) Representative images from the 9 sites imaged for each well in a 96-well plate. The white scale bars represent 100 μm. (B) Quantitative relative fluorescence intensity per cell for cells treated with substrate 4. Error bars represent the standard deviation obtained from triplicates (3 separate wells, 9 sites imaged in each well).

FIG. 16 is a graph showing that fluorescence quenched substrate 4 can quantitatively measure the IC₅₀ value of AT3375 in fixed SK-N-SH cells and replicates values measured in live cells. Error bars represent the standard deviation obtained from triplicates (3 separate wells, 9 sites imaged in each well). Representative images from the 9 sites imaged for each well in a 96-well plate.

FIG. 17 is a graph showing that MDFL1 shows selectivity for hydrolysis by GCase. Comparison of the Michaelis-Menten kinetics of MDFL1 with recombinant GCase (20 nM) and GBA2 (20 nM). Error bars represent standard deviation (n=3).

FIGS. 18A-E shows the investigation of the signal stability of the glucosidase substrate PFB-FDGlu. (A) The chemical structure of PFB-FDGlu; (B) Dose response of PFB-FDGlu in SK-N-SH cells with an incubation time of 1.5 hours; (C) Representative microscopy images of the dose response of PFB-FDGlu in SK-N-SH cells; (D) Time dependent fluorescence signal plot of SK-N-SH cells treated with 500 μM PFB-FDGlu and stopped with 10 μM AT3375; (E) Representative microscopy images of the signal stability experiment at selected time points. Error bars represent the standard error of the mean (SEM) (n=4).

FIGS. 19A-B show the comparison of time and dose response of substrate 1 (also referred to as MDFL1-dML) in trypsinized SK-N-SH cells. (A) Dose response of substrate 1 (also referred to as MDFL1-dML) after a two-hour incubation in trypsinized SK-N-SH cells; (B) Time response of 10 μM Substrate 1 (also referred to as MDFL1-dML) in trypsinized SK-N-SH cells. Error bars represent standard error of the mean (n=3).

FIGS. 20A-B show the IC₅₀ determination of AT3375 in SK-N-SH cells using flow cytometry. (A) Scatter plot of gated SK-N-SH cells at different concentrations of AT3375; (B) Dose response curve of AT3375 measured in SK-N-SH cells using median fluorescence intensity (MFI). Error bars represent standard deviation (n=3).

FIGS. 21A-D show the in vitro characterization of iPSC derived NPCs. (A) Karyotype analysis shows normal chromosome number (46, XY) and absence of large genomic rearrangements, deletions, or duplications; (B) flow cytometry analysis demonstrates the expression of the pluripotency marker protein TRA-1-60; (C) Immunocytochemistry analysis shows the expression of the pluripotency marker protein OCT4; (D) Immunocytochemistry analysis demonstrates the differentiation to three germ layers.

FIGS. 22A-B show the time and dose response of substrate 1 (also referred to as MDFL1-dML) in iPSC derived monocytes and neural progenitor cells. (A) substrate 1 (also referred to as MDFL1-dML) shows linear time and dose response in a microscopy assay of neural progenitor cells; (B) substrate 1 (also referred to as MDFL1-dML) shows linear time and dose response in a flow cytometry assay of monocytes. Error bars represent standard error of the mean (n=3).

FIGS. 23A-B show the in vitro characterization of iPSC derived NPCs. (A) GCase activity in the lysates of NPCs measured using substrate 4MU-Glc. *** p<0.001; ns: not significant; n=3; (B) GCase protein levels in NPCs measured using immunoblot. Densitometry of GCase bands was quantified and normalized to that of β-actin (loading control). Normalized GCase protein level from each line was then divided that of PGPC1 to yield relative GCase level, while relative GCase level of PGPC1 was set arbitrarily as 1. Error bars represent standard error of the mean (n=3).

FIG. 24 shows the ¹H NMR spectrum of compound MDFL1.

FIG. 25 shows the ¹³C NMR spectrum of compound MDFL1.

FIG. 26 shows the 2-D COSY NMR spectrum of compound MDFL1.

FIG. 27 shows the ¹H NMR spectrum of compound substrate 1 (also referred to as MDFL1-dML).

FIG. 28 shows the ¹³C NMR spectrum of compound substrate 1 (also referred to as MDFL1-dML).

FIG. 29 shows the 2-D COSY NMR spectrum of compound substrate 1 (also referred to as MDFL1-dML).

### DETAILED DESCRIPTION

The disclosure provides, in part, fluorescence-quenched β-glucocerebrosidase (GCase) substrates and uses thereof. In some aspects, such compounds may be useful in methods for monitoring GCase activity within cells or tissue, methods for localization of GCase activity within lysosomes, methods for monitoring the effects of a GCase inhibitor in cells or tissue, methods for monitoring the effects of a GCase chaperone in cells or tissue, methods for monitoring the effects of enzyme replacement or augmentation using exogenous GCase, methods for monitoring the effects of gene therapy approaches to augment GCase activity within lysosomes and cells, methods for detecting increased or decreased levels of GCase that may be associated with disease, including for example, Parkinson's disease, methods for monitoring the effects of a GCase activator in cells or tissue, methods for monitoring GCase activity within cells or tissue as a biomarker for a GCase-directed therapy, or methods for conducting a cell-based library screen to identify a GCase activity enhancer. In alternative aspects, such compounds may be useful in identifying GCase chaperones, studying the trafficking and regulation of GCase within cells, screening for endogenous protein modifiers of GCase activity, as well as screening for activators that function within cells to influence GCase activity.

### β-Glucocerebrosidases and Substrates Thereof

By a “β-glucocerebrosidase” or “GCase” is meant an enzyme with glucosylceramidase activity (EC 3.2.1.45) that catalyzes the hydrolytic cleavage of the beta-glucosidic linkage of the glycolipid glucocerebroside (also known as glucosylceramide). Alternative names for a GCase include: acid beta-glucosidase, beta-GC, glucosylceramidase, GlcCerase, D-glucosyl-N-acylsphingosine glucohydrolase, GBA, GBA1, GBA2, and GBA3. In some embodiments, the GCase may be a mammalian GCase, such as a rat, mouse or human GCase. The GCase may be a wild-type GCase or a mutant GCase. In some embodiments, the GCase may be a wild-type mammalian GCase, such as a rat, mouse or human wild-type GCase. In some embodiments, the GCase may be a mutant mammalian GCase, such as a rat, mouse or human mutant GCase. In some embodiments, the GCase may be a human lysosomal GCase. In some embodiments, the GCase may be a human non-lysosomal GCase. In some embodiments, the GCase may be a human cytosolic GCase. In some embodiments, the GCase may have a sequence as set forth in any one of the following Accession numbers: P04062, Q9HCG7, Q9H227, P17439, P97265, Q69ZF3, Q5M868, Q70KH2, Q2KHZ8, Q5R8E3, or Q9BDT0. In alternative embodiments, the GCase may be encoded by a sequence as set forth in any one of the following Accession numbers: NG_009783.1, NP_065995.1, NP_066024.1, NP_001121904.1, NP_001264154.1, NP_766280.2, NP_001121111.1, NP_001013109.2, NP_001005730.1, NM_001046421.2, NM_001134016.1, or NM_001008997.1. In alternative embodiments, the human GCase may have the sequence set forth below:

```
(SEQ ID NO: 1)
                10 20 30 40 50 60
MEFSSPSREE CPKPLSRVSI MAGSLTGLLL LQAVSWASGA RPCIPKSEGY SSVVCVCNAT
                70 80 90 100 110 120
YCDSFDPPTF PALGTFSRYE STRSGRRMEL SMGPIQANHT GTGLLLTLQP EQKFQKVKGF
              130 140 150 160 170 180
GGAMTDAAAL NILALSPPAQ NLLLKSYFSE EGIGYNIIRV PMASCDFSIR TYTYADTPDD
              190 200 210 220 230 240
FQLHNFSLPE EDTKLKIPLI HRALQLAQRP VSLLASPWTS PTWLKINGAV NGKGSLKGQP
              250 260 270 280 290 300
GDIYHQTWAR YFVKFLDAYA EHKLQFWAVT AENEPSAGLL SGYPFQCLGF TPEHQRDFIA
              310 320 330 340 350 360
RDLGPTLANS THHNVRLLML DDQRLLLPHW AKVVLTDPEA AKYVHGIAVH WYLDFLAPAK
              370 380 390 400 410 420
ATLGETHRLF PNTMLFASEA CVGSKFWEQS VRLGSWDRGM QYSHSIITNL LYHVVGWTDW
              430 440 450 460 470 480
NLALNPEGGP NWVRNFVDSP IIVDITKDTF YKQPMFYHLG HFSKFIPEGS QRVGLVASQK
              490 500 510 520 530
NDLDAVALMH PDGSAVVVVL NRSSKDVPLT IKDPAVGFLE TISPGYSIHT YLWRRQ
```

In alternative embodiments, the human GCase may have the nucleic acid sequence of a nucleic acid molecule encoding the sequence set forth in SEQ ID NO: 1.

Examples of mutant human GCase may be mutant enzymes bearing the N370S allele (mutant GCase sequence including:

SEQ ID NO: 2)

QSVRLGSWDRGMQYSHSIITSLLYHVVGWTDWNLALNPEGG;,

allele (mutant GCase sequence including:

SEQ ID NO: 3)

SKFIPEGSQRVGLVASQKNDPDAVALMHPDGSAVVVVLNRS;,

the F213I allele (mutant GCase sequence including:

SEQ ID NO: 4)

GKGSLKGQPGDIYHQTWARYIVKFLDAYAEHKLQFWAVTAE;,

the G202R allele (mutant GCase sequence including:

SEQ ID NO: 5)

PTWLKTNGAVNGKGSLKGQPRDIYHQTWARYFVKFLDAYAE;,

or other mutant alleles.

By a “substrate” or a “GCase substrate” or a “GCase substrate molecule” is meant a molecule containing a beta-glucosidic linkage that can be hydrolytically cleaved by a GCase. By “hydrolytically cleaved” or “hydrolytic cleavage” is meant enzymatic hydrolysis of an ORB group at the anomeric position of a GCase substrate molecule, for example as shown in Scheme A, where ORB is an organic group that may be enzymatically hydrolyzed by a GCase, and RA is an organic group that does not prevent the substrate molecule from being hydrolytically cleaved by a GCase.

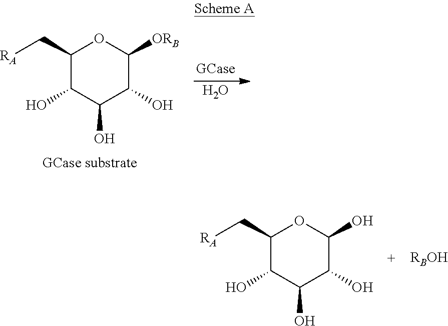

Accordingly, in some embodiments, by “hydrolytically cleaved” is meant enzymatic hydrolysis of a GCase substrate molecule as for example shown in Scheme B:

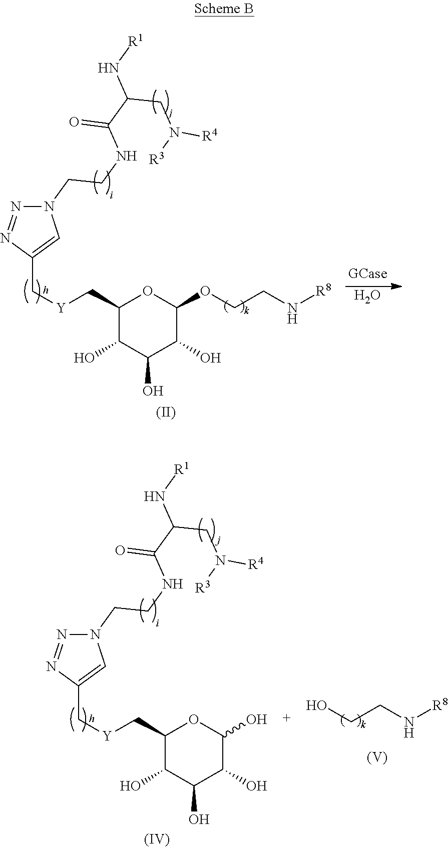

where R¹ may be a suitable fluorophore and R⁸ may be a suitable quencher; R³ and R⁴ may be the same or different and selected from a list of hydrogen, or an alkyl chain of one to 4 carbons or a peptide of between 1 and 10 amino acids in length that is covalently linked using an amide bond, or another suitable bioconjugation reaction, or using a suitable click reaction; or R¹ may be a suitable quencher and R² may be a suitable fluorophore; h may be an integer from 1 to 5; i may be an integer from 1 to 5; j may be a n integer from 1 to 5; k may be an integer from 1 to 10; Y may be an O or S or NH or CH₂. It is to be understood that compounds as described herein, for example, compounds of Formula II, may be similarly hydrolytically cleaved to yield compounds (V) and (VI).

By a “fluorophore” is meant a chemical group that exhibits fluorescence. By “exhibits fluorescence” is meant that the chemical group absorbs light of a specific wavelength (the excitation or absorption wavelength) and re-emits light at a longer wavelength (the emission wavelength). A fluorophore may exhibit an absorption (or excitation) spectrum and an emission spectrum. The wavelength of maximum excitation or absorption for a fluorophore may be any value between about 150 nm to about 800 nm, or in the range of about 300 nm to about 600 nm, or in the range of about 400 to about 600 nm, or any specific value within any of these ranges, such as 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, or 600 nm. In some embodiments, the excitation or absorption wavelength may be 503 nm. The wavelength of maximum emission for a fluorophore may be any value between about 200 nm to about 900 nm, or in the range of about 300 nm to about 700 nm, or in the range of about 400 to about 600 nm, or any specific value within any of these ranges, such as 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630, nm, 640 nm, 650 nm, 660 nm, 670 nm, 680 nm, 690, or 700 nm. In some embodiments, the emission wavelength may be 512 nm.

In some embodiments, a “suitable” fluorophore may be a fluorophore that can be conjugated to an exo-glycosidase substrate, for example as indicated in Scheme C, Scheme D or Scheme E:

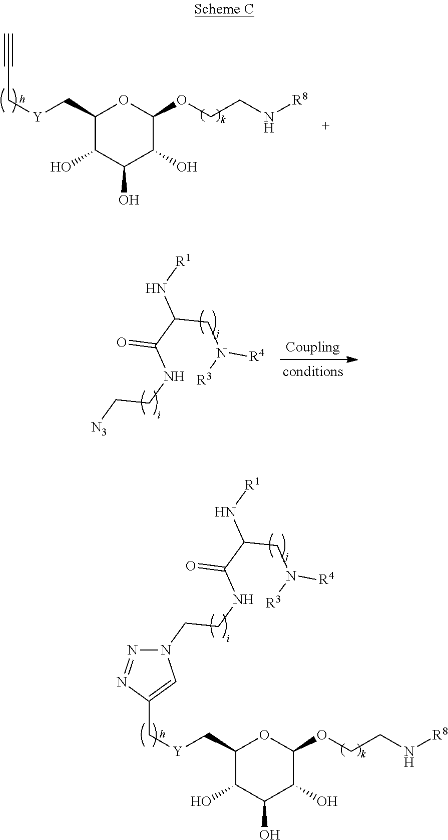

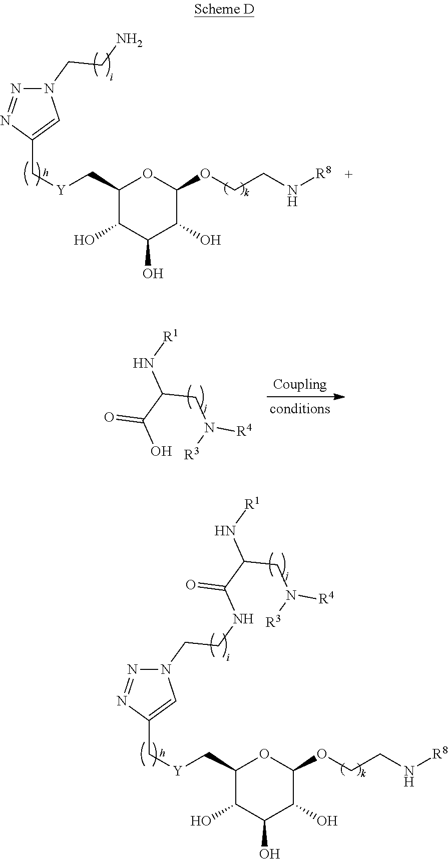

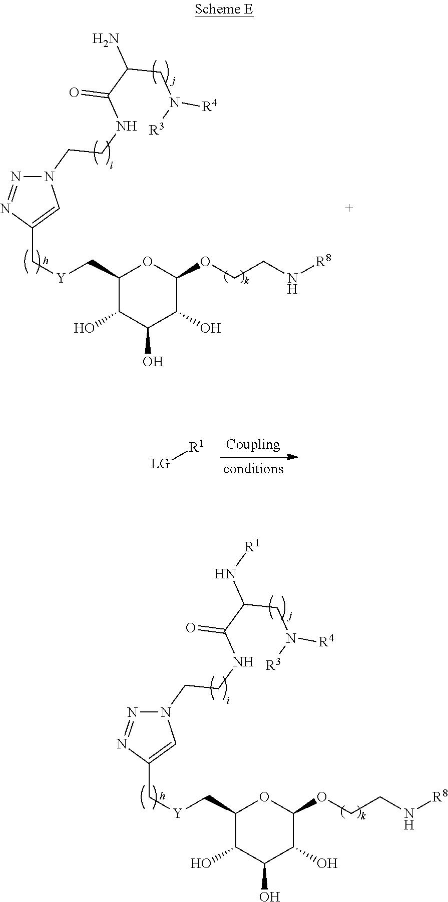

where R¹ may be a suitable fluorophore and R⁸ may be a suitable quencher; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; or R¹ may be a suitable quencher and R² may be a suitable fluorophore; h may be an integer from 1 to 5; i may be an integer from 1 to 5; j may be a n integer from 1 to 5; k may be an integer from 1 to 10; Y may be an O or S or NH or CH₂.

In some embodiments, a “suitable” fluorophore may be a fluorophore having a boron-dipyrromethene (BODIPY®) or a rhodamine core at the R¹ position as indicated in Table 1.

In some embodiments, a suitable fluorophore may include one or more of the following: 5-sulfonaphthalen-1-yl, 5-[(2-azidoethyl)amino] naphthalene-1-sulfonic acid (EDANS), 3-(5,5-difluoro-7-(1H-pyrrol-2-yl)-5H-dipyrrolo[1,2-c:2\l′-f] [1,3,2]diazaborinin-4-ium-5-uid-3-yl)propanoyl, Carboxytetramethylrhodamine (TAMRA), boron-dipyrromethene (BODIPY®) 576/589, BODIPY® FL, BODIPY® R6G, BODIPY® TMR-X, BODIPY® 581/591, BODIPY® TR-X, BODIPY® 630/665-X, FAM, TET, HEX, JOE, VIC, NED, TMR, ROX, TAMRA, CAL Fluor Gold 540, CAL Fluor Orange 560, CAL Red, CAL Orange, CAL Gold, Cy3, Cy3.5, Cy5, Cy5.5, Quasar 570, Quasar 670, Pulsar-650, Oyster 556, Oyster 645, CAL Fluor Red 590, CAL Fluor Red 635, CAL Fluor Red 610, CAL Fluor Red 610, Texas red, Janelia Fluor® 525, Janelia Fluor® 549, Janelia Fluor® 585, Janelia Fluor® 635, Janelia Fluor® 646, Janelia Fluor® 669, LC red 610, LC red 610, LC red 640, LC red 670, LC red 705, Oregon Green 488, Oregon Green 514, Rhodamine Green, Yakima Yellow, Rhodamine Red-X, or Redmond Red, including fluorophores described herein or known in the art.

By a “quencher” is meant a chemical group that absorbs light at, or close to, the emission wavelength of the fluorophore in the fluorescence-quenched substrate, as described herein. In some embodiments, a quencher may be a complementary quencher, that is, a quencher that absorbs energy at, or close to, the emission wavelength of the fluorophore. In some embodiments, a quencher may be a dark quencher, that is, a chemical group that absorbs energy at, or close to, the emission wavelength of the fluorophore and dissipates the energy as heat (i.e. non-radiatively). In some embodiments, a quencher may be a fluorescent quencher, that is, a chemical group that absorbs energy at, or close to, the emission wavelength of the fluorophore and dissipates the energy as light (i.e. the fluorescent quencher absorbs light at the emission wavelength of a suitable fluorophore and re-emits light at a longer wavelength). In some embodiments, a “suitable” quencher may be a quencher that can be conjugated to a GCase substrate, for example as indicated in Scheme F or Scheme G:

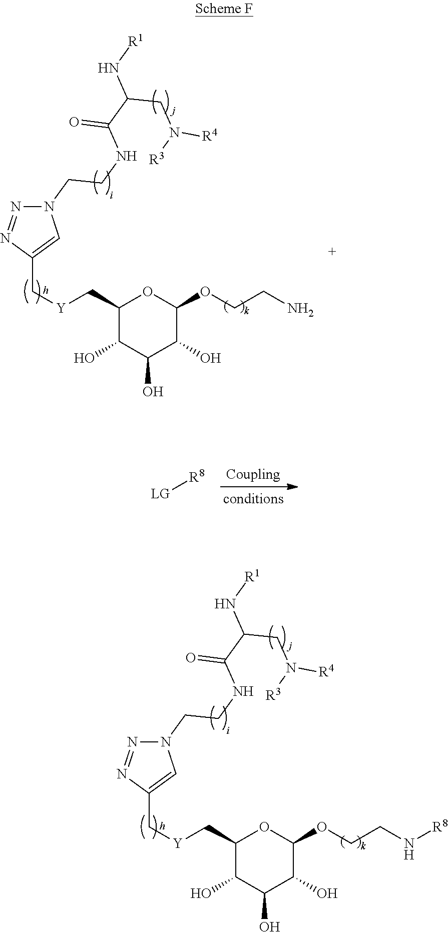

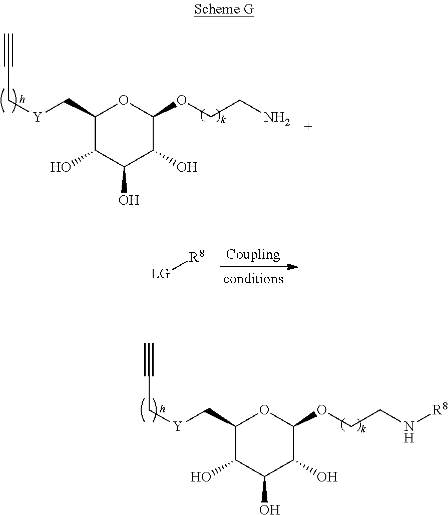

where R¹ may be a suitable fluorophore and R⁸ may be a suitable quencher; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; or R¹ may be a suitable quencher and R⁸ may be a suitable fluorophore; h may be an integer from 1 to 5; i may be an integer from 1 to 5; j may be a n integer from 1 to 5; k may be an integer from 1 to 10; Y may be an O or S or NH or CH₂.

In some embodiments, a “suitable” quencher may a 4-((4-((E)-(2-methoxy-5-methyl-4-((E)-(4-methyl-2-nitrophenyl)diazenyl)phenyl)diazenyl)phenyl)(methyl)amino)butanoyl (BHQ®1)or a 4-((4-((E)-(2,5-dimethoxy-4-((E)-(4-nitrophenyl)diazenyl)phenyl)diazenyl)phenyl)(methyl)amino)butanoyl (BHQ®2) group at the R² position, as indicated in Table 1.

In some embodiments, a “suitable quencher” may include one or more of the following: dimethylaminoazobenzenesulfonic acid, (DABCYL) 4-([4-(dimethylamino)phenyl]azo)-benzoic acid (DABCYL), (E)-4-((4-(dimethylamino)phenyl)diazenyl)benzoyl, 4-((4-((E)-(2,5-dimethoxy-4-((E)-(4-nitrophenyl)diazenyl)phenyl)diazenyl)phenyl)(methyl)amino)-butanoyl, Black Hole Quencher® 2 (BHQ®2), DDQ-I, DDQ-II, Eclipse, ElleQuencher, Iowa Black FQ, Iowa Black RQ, BHQ®0, BHQ®1, BHQ®3, QSY-7, QSY 9, QSY-21, or QSY 35, including quenchers described herein or known in the art. A discussion of fluorophore and quencher groups may be found, for example, in “The Molecular Probes Handbook, A Guide to Fluorescent Probes and Labeling Technologies” I. Johnson and M.T.Z. Spence (eds.), Eleventh Edition (Life Technologies, 2010).

By “not substantially fluorescent” is meant a molecule that exhibits efficient internal quenching. By “efficient internal quenching” or “efficient quenching” or “internal quenching” is meant decreased intensity of fluorescent emission due to the presence of the quencher within the substrate molecule. The decrease in fluorescent emission may be a decrease by any value between about 10% and about 100%, or of any value between about 30% and about 60%, or about 100%, or a decrease by about 1-fold, 2-fold, 5-fold, 10-fold, 100-fold, 1000-fold, 10,000-fold, 100,000-fold, or more, or by about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, 99.99990%, or more in comparison to a reference sample or compound, or in comparison to a cleavage product molecule such as, for example, either a compound of Formula (I) or a compound of Formula (II). It is to be understood that the decrease in fluorescent emission does not require full absence of fluorescent emission. Examples of a suitable reference compound or control is, for example, BODIPY® FL.

A cleavage product molecule for a fluorescence-quenched substrate such as, for example, a compound of Formula (III)-(VI) may exhibit significant fluorescent emission. By “significant fluorescent emission” is meant a fluorescent intensity in the range of about 0.1 to about 1×10 9 relative fluorescence units (RFU), or in the range of about 10 RFU to about 1×10 8 RFU, or in the range of about 100 RFU to about 1×10 6 RFU, or in the range of about 100 RFU to about 5,000 RFU, or any specific fluorescent intensity within any of these ranges, such as 50 RFU, 60 RFU, 70 RFU, 80 RFU, 90 RFU, 100 RFU, 110 RFU, 120 RFU, 130 RFU, 140 RFU, 150 RFU, 160 RFU, 170 RFU, 180 RFU, 190 RFU, 200 RFU, 300 RFU, 400 RFU, 500 RFU, 600 RFU, 700 RFU, 800 RFU, 900 RFU, 1,000 RFU, 2,000 RFU, 3,000 RFU, 4,000 RFU, 5,000 RFU, 10,000 RFU, 50,000 RFU, 100,000 RFU, 500,000 RFU, 1×10 6 RFU, 0.5×10 7 RFU, 1×10 7 RFU, 0.5×10 8 RFU, 1×10 8 RFU, 0.5×10 9 RFU, 1×10 9 RFU, or any value within or about the described range. The fluorescent intensity may be measured based on concentration of cleavage product molecule, number of cells, amount of tissue, or any other suitable unit for measuring fluorescent intensity.

In alternative embodiments, one or more of the compounds according to the disclosure may be specifically cleaved by one isoform of an exo-glycosidase, for example the human lysosomal GBA1 isoform. In alternative embodiments, one or more of the compounds according to the disclosure may be specifically cleaved by the human lysosomal GBA1 isoform over the human non-lysosomal GBA2 isoform and/or the human cytosolic GBA3 isoform. By “specifically hydrolytically cleaved” or “specifically cleaved” is meant a compound that is hydrolytically cleaved by an exo-glycosidase but is not substantially hydrolytically cleaved by other enzymes in a sample, such as a lactase, a sucrase, an isomaltase, an alpha-glucosidase II, a glycogen phosphorylase, an acid alpha-glucosidase, an alpha-galactosidase, a beta-galactosidase, a beta-hexosaminidase, an O-GlcNAcase, or another exo-glycosidase isoform. By “not substantially hydrolytically cleaved” is meant a substrate specificity in the range of about 2-fold to about 100,000-fold, or about 10-fold to about 100,000-fold, or in the range of about 100-fold to about 100,000-fold, or in the range of about 1000-fold to about 100,000-fold, or at least about 2-fold, 3-fold, 4-fold, 5-fold, 10-fold, 20-fold, 50-fold, 100-fold, 200-fold, 500-fold, 1000-fold, 1500-fold, 2000-fold, 2500-fold, 3000-fold, 3500-fold, 4000-fold, 4500-fold, 5000-fold, 6000-fold, 7000-fold, 10,000-fold, 25,000-fold, 50,000-fold, 75,000-fold, or any value within or about the described range, where “substrate specificity” means the ratio of the respective k_{cat}/Kₘ constants, that is,

$$[ {k}_{c a t ( e x o - g l u cosidase )} / {K}_{m ( e x o - g l u c o s i d a s e )} ] / [ {k}_{c a t ( other enzyme )} / {K}_{m ( o ther enzyme )} ]$$

where “k_{cat(exo-glucosidase)}” is the rate constant for cleavage of a substrate molecule by an exo-glucosidase, “K_{m(exo-glucosidase)}” is the Michaelis constant for a substrate molecule and an exo-glycosidase, “k_{cat(other enzyme)}” is the rate constant for cleavage of a substrate molecule by another enzyme, and “K_{m(other enzyme)}” is the Michaelis constant for a substrate molecule and another enzyme.

“Alkyl” refers to a straight (linear) or branched hydrocarbon chain group consisting solely of carbon and hydrogen atoms, containing no unsaturation and including, for example, from one to ten carbon atoms, such as 1, 2, 3, 4, 5, 6, 7, 8, 9 or 10 carbon atoms, and which is attached to the rest of the molecule by a single bond.

A “peptide” is generally two or more amino acids, including naturally occurring or non-naturally occurring amino acids or amino acid analogues, regardless of post-translational modification (e.g., glycosylation or phosphorylation). As used herein, a peptide may include a single amino acid. A peptide may include abnormal linkages, cross links and end caps, non-peptidyl bonds or alternative modifying groups. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the core peptidic structure).

Suitable bioconjugation reactions are known in the art, as for example, set out in McKay et al. (41). It is to be understood that any suitable bioconjugation reaction may be used and the reactions set forth in McKay et al. are for the purpose of exemplification.

Suitable click reactions are known in the art, as for example, set out in Lang et al. (42). It is to be understood that any suitable click reaction may be used and the reactions set forth in reference 42 are for the purpose of exemplification.

In specific embodiments, the disclosure provides compounds described generally by Formula (I), (II), (IIa)-(IIl), (III), (IIIa)-(IIIe) and salts thereof.

As set forth in Formula (I):

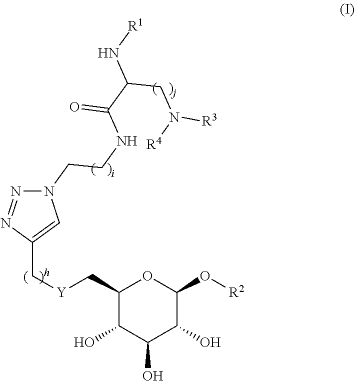

R¹ may be a suitable fluorophore or a suitable quencher;
R² may be

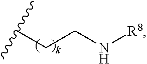

where k may be an integer from 1 to 10, and when R¹ is a suitable fluorophore, R¹ may be a suitable quencher; or when R¹ is a suitable quencher, R⁸ may be a suitable fluorophore, or
R² may be

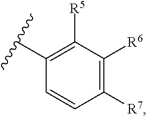

where R⁵, R⁶ and R⁷ may each independently be H, F, NO₂, or a suitable quencher;
R³ and R⁴ may each independently be H, an alkyl chain of one to 4 carbons, or a peptide of between 1 and 10 amino acids in length that is covalently linked using an amide bond, or another suitable bioconjugation reaction, or using a suitable click reaction;
h may be an integer from 1 to 5;
i may be an integer from 1 to 5;
j may be a n integer from 1 to 5; and
Y may be O, S, NH or CH₂.

As set forth in Formula (II):

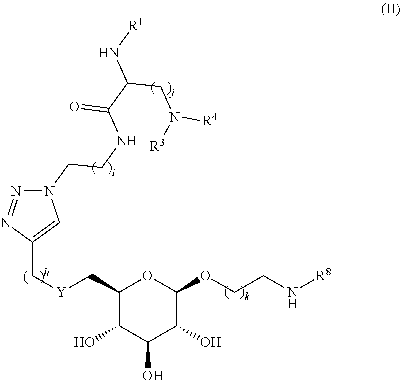

R¹ may be a suitable fluorophore and R⁸ may be a suitable quencher; or R¹ may be a suitable quencher and R⁸ may be a suitable fluorophore; R³ and R⁴ may be the same or different and selected from a list of hydrogen, an alkyl chain of one to 4 carbons, or a peptide of between 1 and 10 amino acids in length that is covalently linked using an amide bond, or another suitable bioconjugation reaction, or using a suitable click reaction; h may be an integer from 1 to 5; i may be an integer from 1 to 5; j may be a n integer from 1 to 5; k may be an integer from 1 to 10; Y may be an O or S or NH or CH₂.

In some embodiments, Y as set forth in Formula (II) may be O or S or NH or CH₂.

In some embodiments, Y may be O.

In some embodiments, R¹ as set forth in Formula (II) may be a suitable fluorophore and R⁸ as set forth in Formula (II) may be a suitable quencher.

In some embodiments, R¹ as set forth in Formula (II) may be a suitable quencher and R⁸ as set forth in Formula (II) may be a suitable fluorophore.

In some embodiments, R³ as set forth in Formula (II) may be H or an alkyl chain of one to 4 carbons or a peptide of between 1 and 10 amino acids in length that is covalently linked using an amide bond, or another suitable bioconjugation reaction, or using a suitable click reaction. In some embodiments, R³ may be CH₃.

In some embodiments, R⁴ as set forth in Formula (II) may be H or an alkyl chain of one to 4 carbons or a peptide of between 1 and 10 amino acids in length that is covalently linked using an amide bond, or another suitable bioconjugation reaction, or using a suitable click reaction. In some embodiments, R⁴ may be CH₃.

In some embodiments, h as set forth in Formula (II) may be an integer from 1 to 5. In some embodiments, h may be 1, 2, 3, 4, or 5. In some embodiments, h may be 1.

In some embodiments, i as set forth in Formula (II) may be an integer from 1 to 5.

In some embodiments, i may be 1, 2, 3, 4, or 5. In some embodiments, i may be 2.

In some embodiments, j as set forth in Formula (II) may be an integer from 1 to 5.

In some embodiments, j may be 1, 2, 3, 4, or 5. In some embodiments, j may be 4.

In some embodiments, k as set forth in Formula (II) may be an integer from 1 to 10. In some embodiments, k may be 1, 2, 3, 4, 5, 6, 7, 8, 9, or 10. In some embodiments, k may be 2.

In specific embodiments, compounds according to Formula (II) include compounds 1,2, and 4 described in Table 1.

In alternative embodiments, the disclosure provides a compound of Formula (IIa), or an acceptable salt thereof:

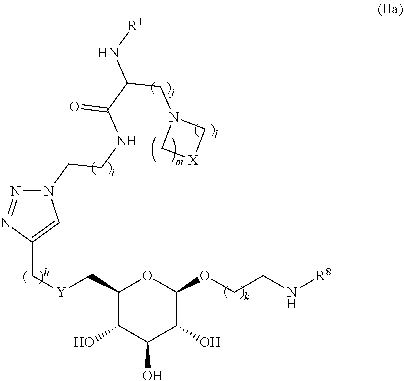

where R¹ may be a suitable fluorophore and R⁸ may be a suitable quencher; or R¹ may be a suitable quencher and R⁸ may be a suitable fluorophore; h may be an integer from 1 to 5; i may be an integer from 1 to 5; j may be a n integer from 1 to 5; k may be an integer from 1 to 10; m may be an integer from 1 to 4; 1 may be an integer from 1 to 4; and X may be O or S or NH or CH₂ or NR⁹ where R⁹ may be a short alkyl chain of 1 to 4 carbon atoms; and Y may be O or S or NH or CH₂.

In alternative embodiments, the disclosure provides a compound of Formula (IIb), or an acceptable salt thereof:

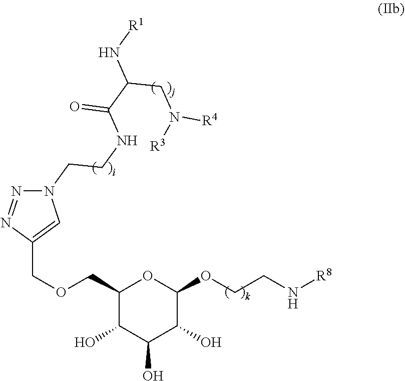

where R¹ may be a suitable fluorophore and R⁸ may be a suitable quencher; or R¹ may be a suitable quencher and R⁸ may be a suitable fluorophore; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; i may be an integer from 1 to 5; j may be an integer from 1 to 5; k may be an integer from 1 to 10.

In alternative embodiments, the disclosure provides a compound of Formula (IIc), or an acceptable salt thereof.

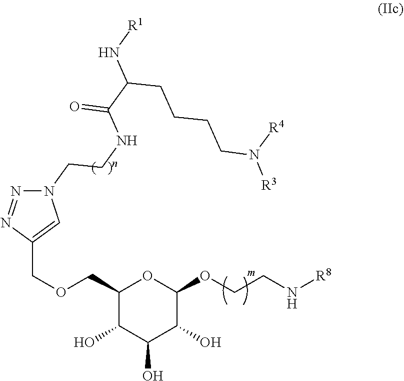

where R¹ may be a suitable fluorophore and R⁸ may be a suitable quencher; or R¹ may be a suitable quencher and R² may be a suitable fluorophore; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; n may be an integer from 1 to 10; and m may be an integer from 1 to 5.

In alternative embodiments, the disclosure provides a compound of Formula (IId), or an acceptable salt thereof.

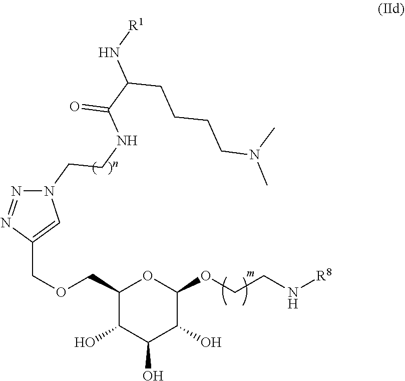

where R¹ may be a suitable fluorophore and R⁸ may be a suitable quencher; or R¹ may be a suitable quencher and R⁸ may be a suitable fluorophore; n may be an integer from 1 to 10; and m may be an integer from 1 to 5.

In alternative embodiments, the disclosure provides a compound of Formula (IIe), or an acceptable salt thereof.

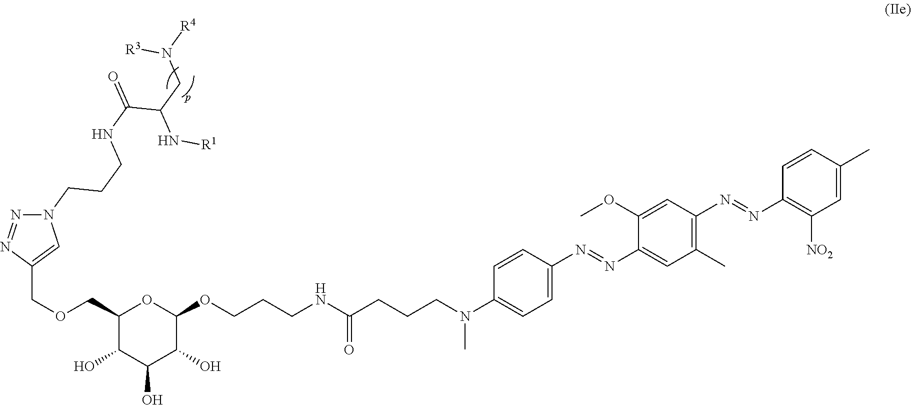

where R¹ may be a suitable fluorophore; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; n may be an integer from 1 to 10; and m may be an integer from 1 to 5; p may be an integer from 1 to 5.

In alternative embodiments, the disclosure provides a compound of Formula (IIf), or an acceptable salt thereof

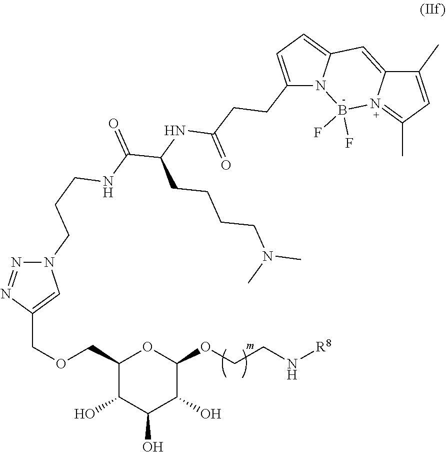

where R⁸ may be a suitable quencher; m may be an integer from 1 to 5.

In alternative embodiments, the disclosure provides a compound of Formula (IIg), or an acceptable salt thereof:

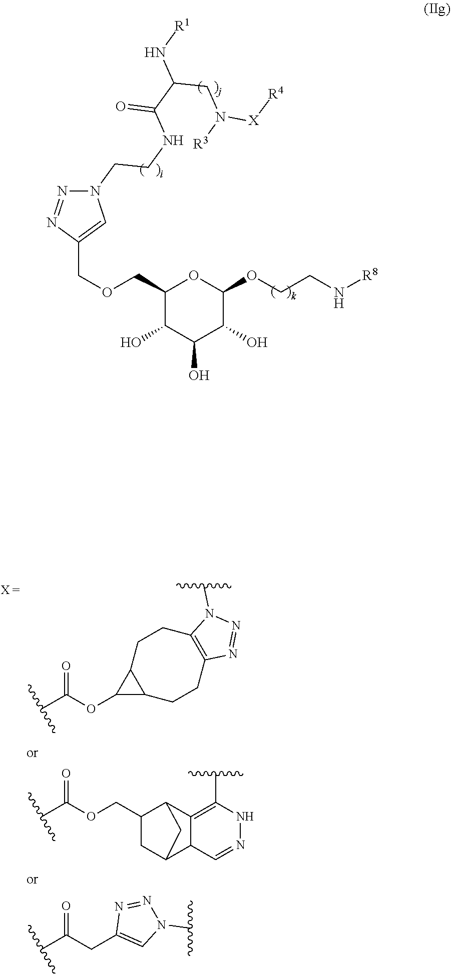

where R¹ may be a suitable fluorophore and R⁸ may be a suitable quencher; or R¹ may be a suitable quencher and R⁸ may be a suitable fluorophore; R³ may be selected from a list of hydrogen, an alkyl chain of one to 4 carbons; and R⁴ may be a peptide between 1 and 10 amino acid residues in length; X may be the product of a click reaction; i may be an integer from 1 to 5; j may be a n integer from 1 to 5; k may be an integer from 1 to 10.

In alternative embodiments, the disclosure provides a compound of Formula (IIh), or an acceptable salt thereof:

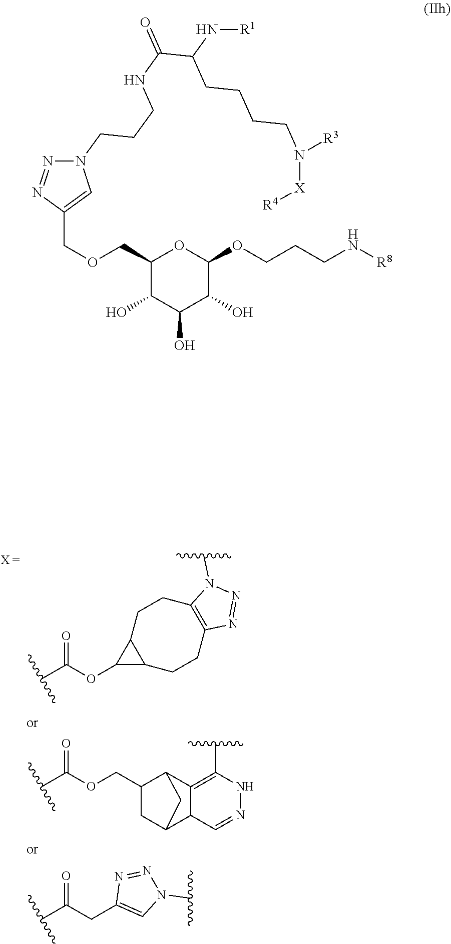

where R¹ may be a suitable fluorophore and R⁸ may be a suitable quencher; or R¹ may be a suitable quencher and R⁸ may be a suitable fluorophore; R³ may be selected from a list of hydrogen, an alkyl chain of one to 4 carbons; R⁴ may be a peptide between 1 and 10 amino acid residues in length; X may be the product of a click reaction.

In alternative embodiments, the disclosure provides a compound of Formula (IIi), or an acceptable salt thereof:

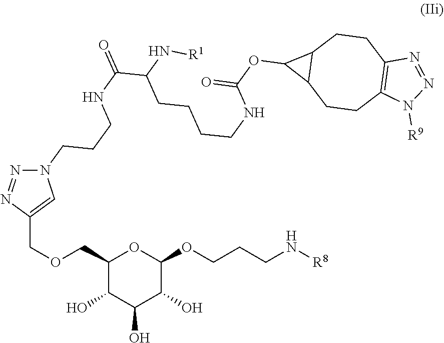

where R¹ may be a suitable fluorophore and R⁸ may be a suitable quencher; or R¹ may be a suitable quencher and R⁸ may be a suitable fluorophore; R⁹ may be an oligopeptide between 1 and 10 amino acid residues in length.

In alternative embodiments, the disclosure provides a compound of Formula (IIj), or an acceptable salt thereof:

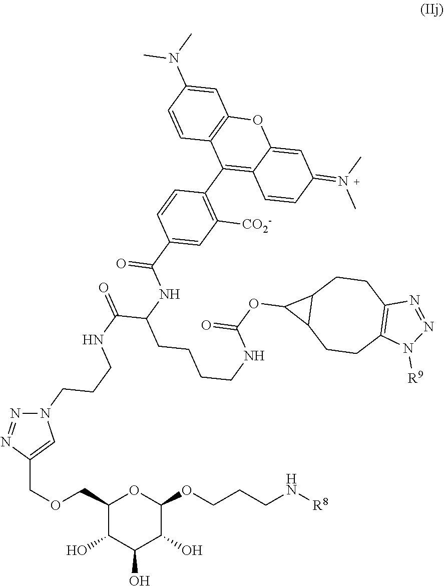

where R² may be a suitable quencher; R⁹ may be an oligopeptide between 1 and 10 amino acids in length.

In alternative embodiments, the disclosure provides a compound of Formula (IIk), or an acceptable salt thereof:

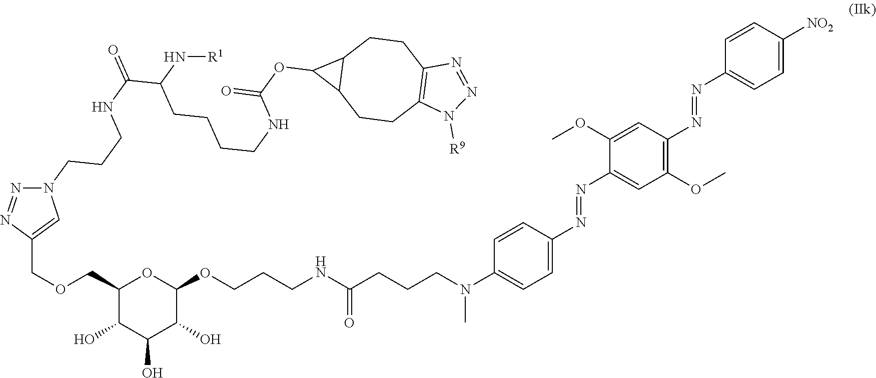

where R¹ may be a suitable fluorophore; R⁹ may be an oligopeptide between 1 and 10 amino acid residues in length.

In alternative embodiments, the disclosure provides a compound of Formula (IIl), or an acceptable salt thereof:

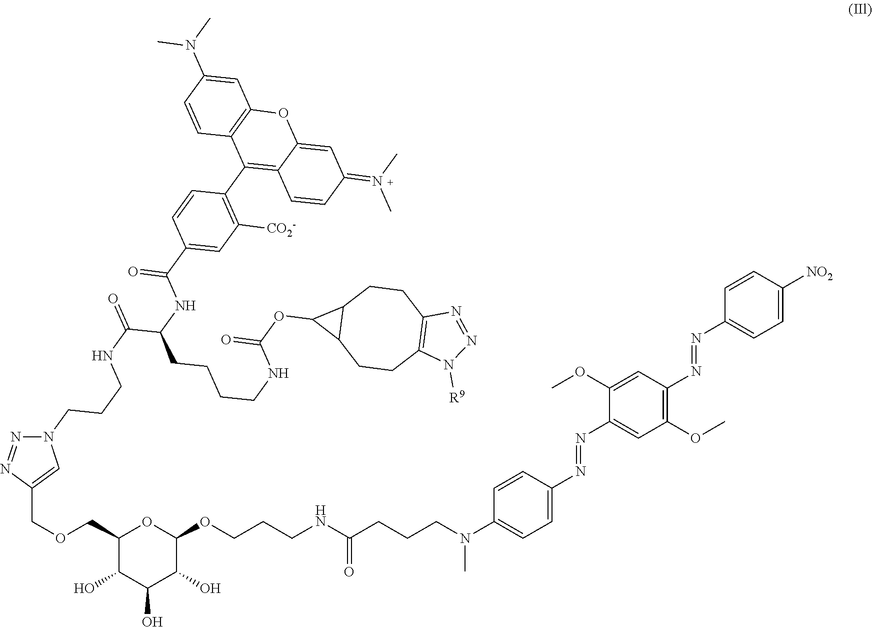

where R⁹ may be an oligopeptide between 1 and 10 amino acid residues in length.

In alternative embodiments, the disclosure provides a compound as set forth in Formula (III):

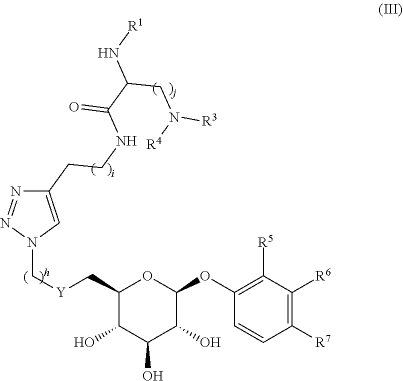

R¹ may be a suitable fluorophore; R³ and R⁴ may be the same or different and selected from a list of hydrogen, or an alkyl chain of one to 4 carbons; R⁵, R⁶ and R⁷ may be the same or different and selected from a list of hydrogen, fluorine, NO₂ group, or a suitable quencher; h may be an integer from 1 to 5; i may be an integer from 1 to 5; j may be a n integer from 1 to 5; Y may be an O or S NH or CH₂.

In some embodiments, Y as set forth in Formula (III) may be 0 or S or NH or CH₂. In some embodiments, Y may be 0.

In some embodiments, R¹ as set forth in Formula (III) may be a suitable fluorophore and R⁵, R⁶ and R⁷ as set forth in Formula (III) may be a suitable quencher.

In some embodiments, R¹ as set forth in Formula (III) may be a suitable quencher and R⁵, R⁶ and R⁷ as set forth in Formula (III) may be a suitable fluorophore.

In some embodiments, R³ as set forth in Formula (III) may be H or an alkyl chain of one to 4 carbons or a peptide of between 1 and 10 amino acids in length that is covalently linked using an amide bond, or another suitable bioconjugation reaction, or using a suitable click reaction. In some embodiments, R³ may be CH₃.

In some embodiments, R⁴ as set forth in Formula (III) may be H or an alkyl chain of one to 4 carbons or a peptide of between 1 and 10 amino acids in length that is covalently linked using an amide bond, or another suitable bioconjugation reaction, or using a suitable click reaction. In some embodiments, R⁴ may be CH₃.

In some embodiments, R⁵, R⁶ and R⁷ may be may be the same or different and selected from a list of hydrogen, fluorine, NO₂ group, R⁴,R⁵ may be hydrogen atoms.

In some embodiments, h as set forth in Formula (III) may be an integer from 1 to 5. In some embodiments, h may be 1, 2, 3, 4, or 5. In some embodiments, h may be 1.

In some embodiments, i as set forth in Formula (III) may be an integer from 1 to 5. In some embodiments, i may be 1, 2, 3, 4, or 5. In some embodiments, i may be 2.

In some embodiments, j as set forth in Formula (III) may be an integer from 1 to 5. In some embodiments, j may be 1, 2, 3, 4, or 5. In some embodiments, j may be 4.

In specific embodiments, compounds according to Formula (III) include compound 3 described in Table 1.

TABLE 1

| Example | Structure/Name |
|---|---|
| 1 | 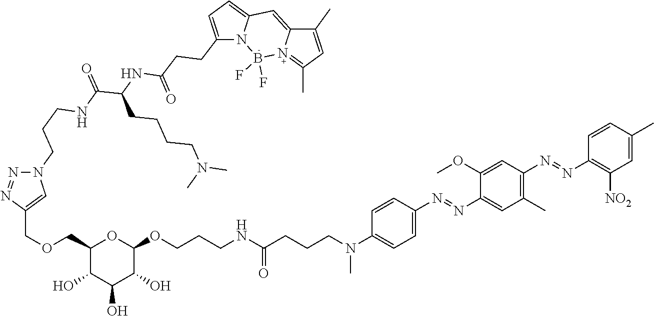 |
| | Name: (S)-2-(3-(5,5-difluoro-7,9-dimethyl-dipyrrolo[1,2-c:2′,1′- |
| | f][1,3,2]diazaborinin-4-ium-5-uid-3-yl)propanamido)-6-(dimethylamino)-N- |
| | (3-(4-((((2R,3S,4S,5R,6R)-3,4,5-trihydroxy-6-(3-(4-((4-((E)-(2-methoxy-5- |
| | methyl-4-((E)-(4-methyl-2- |
| | nitrophenyl)diazenyl)phenyl)diazenyl)phenyl)(methyl)amino)butanamido) |
| | propoxy)tetrahydro-2H-pyran-2-yl)methoxy)methyl)-1H-1,2,3-triazol-1- |
| | yl)propyl)hexanamide |
| 2 | 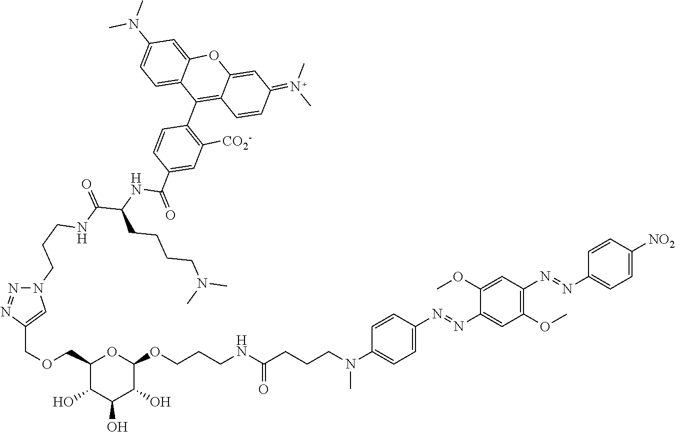 |
| | Name: 5-(((S)-1-((3-(4-((((2R,3S,4S,5R,6R)-6-(3-(4-((4-((E)-(2,5-dimethoxy- |
| | 4-((E)-(4- |
| | nitrophenyl)diazenyl)phenyl)diazenyl)phenyl)(methyl)amino)butanamido) |
| | propoxy)-3,4,5-trihydroxytetrahydro-2H-pyran-2-yl)methoxy)methyl)-1H-1,2,3- |
| | triazol-1-yl)propyl)amino)-6-(dimethylamino)-1-oxohexan-2-yl)carbamoyl)-2- |
| | (6-(dimethylamino)-3-(dimethyliminio)-3H-xanthen-9-yl)benzoate |
| 3 | 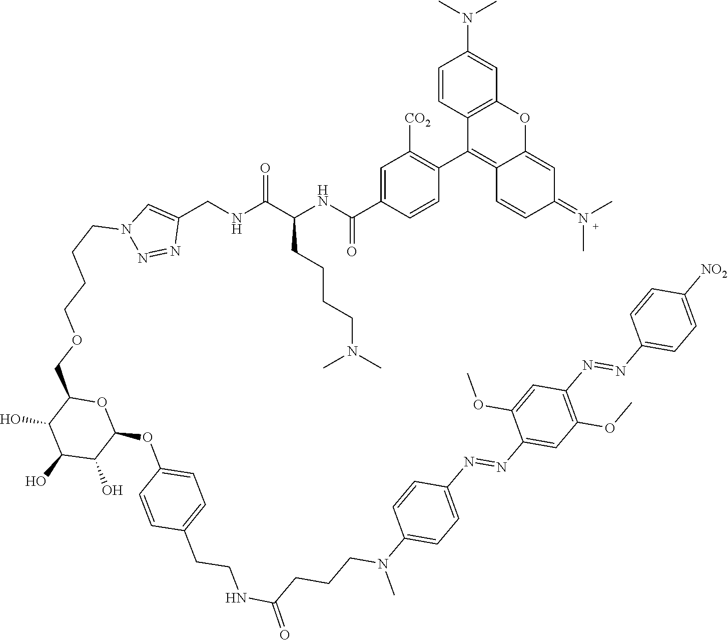 |
| | Name: 5-(((S)-1-(((1-(4-(((2R,3S,4S,5R,6S)-6-(4-(2-(4-((4-((E)-(2,5- |
| | dimethoxy-4-((E)-(4- |
| | nitrophenyl)diazenyl)phenyl)diazenyl)phenyl)(methyl)amino)butanamido)ethyl) |
| | phenoxy)-3,4,5-trihydroxytetrahydro-2H-pyran-2-yl)methoxy)butyl)-1H- |
| | 1,2,3-triazol-4-yl)methyl)amino)-6-(dimethylamino)-1-oxohexan-2- |
| | yl)carbamoyl)-2-(6-(dimethylamino)-3-(dimethyliminio)-3H-xanthen-9- |
| | yl)benzoate |
| 4 | 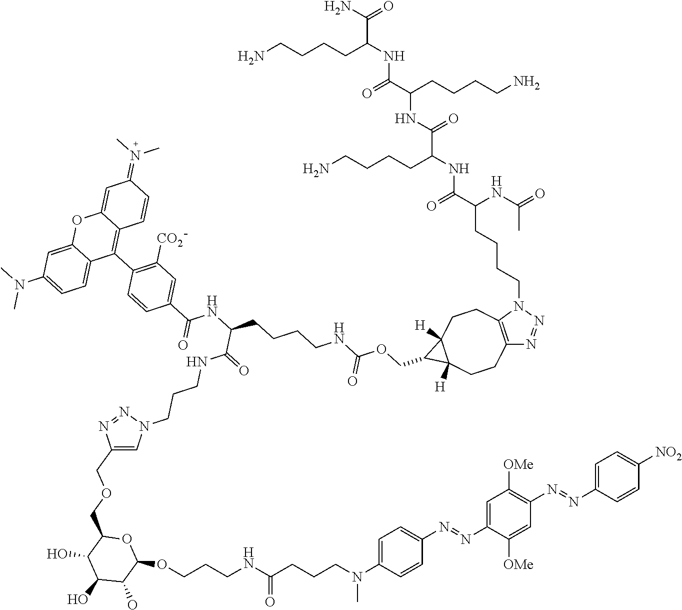 |
| | Name: 5-(((2S)-6-(((((5aS,6R,6aR)-1-((S)-5-acetamido-6-(((S)-6-amino-1- |
| | (((S)-6-amino-1-(((S)-1,6-diamino-1-oxohexan-2-yl)amino)-1-oxohexan-2-yl)amino)- |
| | 1-oxohexan-2-yl)amino)-6-oxohexyl)-1,4,5,5a,6,6a,7,8- |
| | octahydrocyclopropa[5,6]cycloocta[1,2-d][1,2,3]triazol-6- |
| | yl)methoxy)carbonyl)amino)-1-((3-(4-((((2R,3S,4S,5R,6R)-6-(3-(4-((4-((E)- |
| | (2,5-dimethoxy-4-((E)-(4- |
| | nitrophenyl)diazenyl)phenyl)diazenyl)phenyl)(methyl)amino)butanamido) |
| | propoxy)-3,4,5-trihydroxytetrahydro-2H-pyran-2-yl)methoxy)methyl)-1H-1,2,3- |
| | triazol-1-yl)propyl)amino)-1-oxohexan-2-yl)carbamoyl)-2-(6-(dimethylamino)- |
| | 3-(dimethyliminio)-3H-xanthen-9-yl)benzoate |

In alternative embodiments, the disclosure provides a compound of Formula (IIIa), or an acceptable salt thereof.

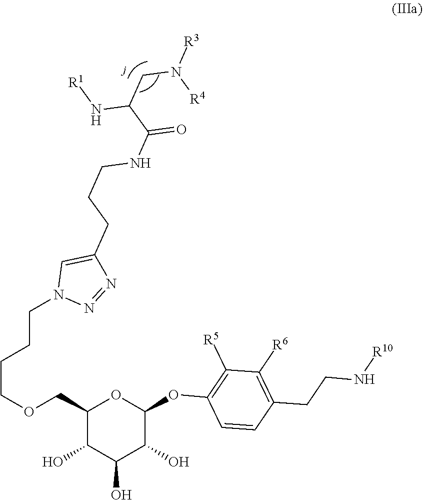

where R¹ may be a suitable fluorophore; R³ and R⁴ may each independently be H, an alkyl chain of one to 4 carbons or a peptide of between 1 and 10 amino acids in length that is covalently linked using an amide bond, or another suitable bioconjugation reaction, or using a suitable click reaction; R⁵ and R⁶ may each independently be H, F, or NO₂; R¹⁰ may be a suitable quencher; j may be a n integer from 1 to 5.

In alternative embodiments, the disclosure provides a compound of Formula (IIIb), or an acceptable salt thereof.

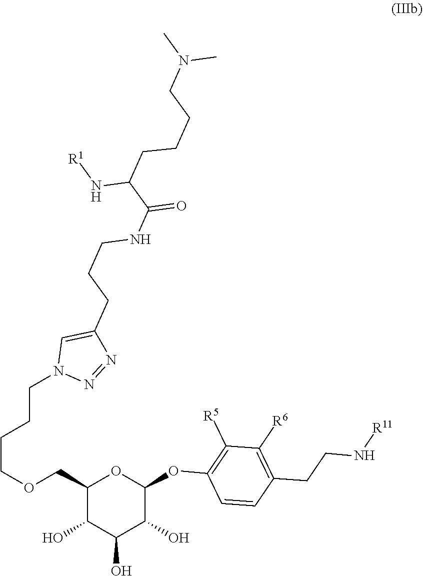

where R¹ may be a suitable fluorophore; R⁵ and R⁶ may each independently H, F, or NO₂; R¹¹ may be a suitable quencher.

In alternative embodiments, the disclosure provides a compound of Formula (IIIc), or an acceptable salt thereof.

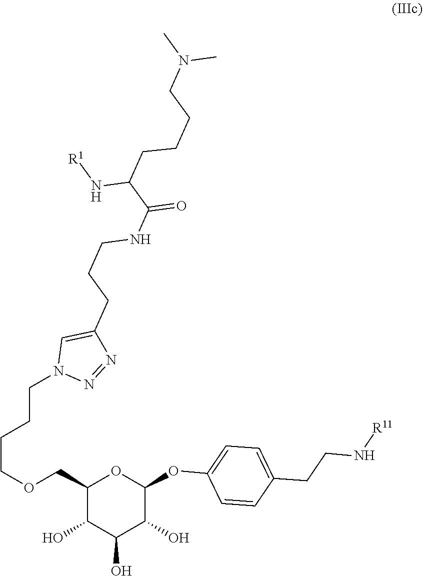

where R¹ may be a suitable fluorophore and R¹¹ may be a suitable quencher.

In alternative embodiments, the disclosure provides a compound of Formula (IIId), or an acceptable salt thereof.

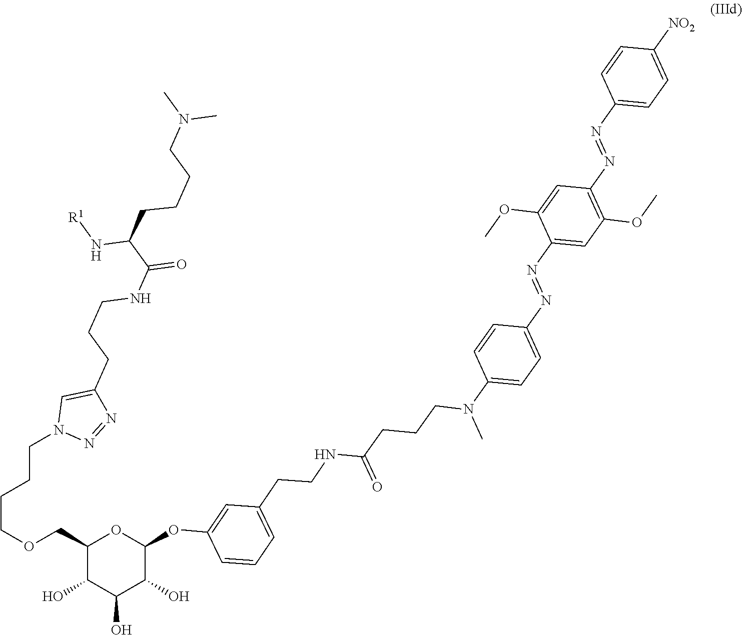

where R¹ may be a suitable fluorophore.

In alternative embodiments, the disclosure provides a compound of Formula (IIIe), or an acceptable salt thereof

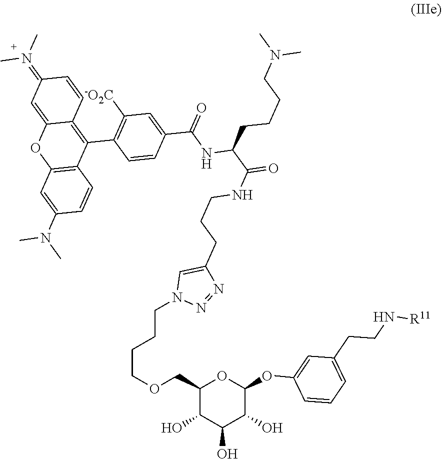

where R¹¹ may be a suitable quencher.

A compound of the present disclosure may be used in the form of a salt. In such cases, compositions in accordance with this disclosure may comprise a salt of such a compound, preferably a physiologically-acceptable salt, which are known in the art. In some embodiments, an “acceptable salt” as used herein means an active ingredient comprising compounds of Formula (I) or Formula (II) used in the form of a salt thereof, particularly where the salt form confers on the active ingredient improved solubility, bioavailability or cell permeability properties as compared to the free form of the active ingredient or other previously disclosed salt form.

An “acceptable salt” may include both acid and base addition salts. An “acceptable acid addition salt” refers to those salts which retain the biological effectiveness and properties of the free bases, which are not biologically or otherwise undesirable, and which may be formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

An “acceptable base addition salt” refers to those salts which may retain the biological effectiveness and properties of the free acids, which may not be biologically or otherwise undesirable. These salts may be prepared from addition of an inorganic base or an organic base to the free acid. Salts derived from inorganic bases may include, but are not limited to, the sodium, potassium, lithium, ammonium, calcium, magnesium, iron, zinc, copper, manganese, aluminum salts and the like. Preferred inorganic salts may be the ammonium, sodium, potassium, calcium, and magnesium salts. Salts derived from organic bases may include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine,methylglucamine, theobromine, purines, piperazine, piperidine, N-ethylpiperidine, polyamine resins and the like. Particularly preferred organic bases may be isopropylamine, diethylamine, ethanolamine, trimethylamine, dicyclohexylamine, choline and caffeine.

Thus, the term “acceptable salt” encompasses all acceptable salts including but not limited to acetate, lactobionate, benzenesulfonate, laurate, benzoate, malate, bicarbonate, maleate, bisulfate, mandelate, bitartarate, mesylate, borate, methylbromide, bromide, methylnitrite, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutame, stearate, glycollylarsanilate, sulfate, hexylresorcinate, subacetate, hydradamine, succinate, hydrobromide, tannate, hydrochloride, tartrate, hydroxynaphthoate, teoclate, iodide, tosylate, isothionate, triethiodide, lactate, panoate, valerate, and the like.

Acceptable salts of a compound of the present disclosure may be used for modifying solubility or hydrolysis characteristics. Also, salts of a compound of this disclosure may include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

The compounds of the present disclosure may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this disclosure. Any formulas, structures or names of compounds described in this specification that do not specify a particular stereochemistry are meant to encompass any and all existing isomers as described above and mixtures thereof in any proportion. When stereochemistry is specified, the disclosure is meant to encompass that particular isomer in pure form or as part of a mixture with other isomers in any proportion.

Fluorescence-quenched substrates, such as compounds according to the disclosure, may be prepared using standard techniques as described herein or known in the art.

### Methods for Use of Fluorescence-Quenched Substrates

A fluorescence-quenched GCase substrate, for example, a GCase substrate which would enable the enzyme to turn over multiple molecules, may be used to assess the enzymatic activity of a GCase within a live or fixed cell or tissue. Accordingly, the current disclosure provides, in part, efficiently quenched fluorescent GCase substrate compounds that enable localization and quantification of enzyme activity in a live or fixed cell or tissue by, for example, imaging.

In some aspects, a compound according to the present disclosure may be useful for determining GCase activity within a cell. By “determining” is meant analysing the effect of a test compound, such as a compound according to the present disclosure, on a test system, such as a cell or tissue. The analysing may be performed, without limitation, using imaging techniques or any other methods described herein or known to those skilled in the art. For example, a test cell may be contacted with a compound according to the present disclosure (e.g., a fluorescence-quenched GCase substrate) under conditions suitable for hydrolytic cleavage of the fluorescence-quenched GCase substrate by a GCase. The intensity of the fluorescence emission (the “fluorescence intensity”) of the test cell may be determined using standard techniques. The fluorescence intensity of the test cell may be compared to that of a control cell (e.g., a cell that has not been exposed to, or contacted with, a compound according to the present disclosure) to determine background or non-specific fluorescence. The difference between the fluorescence intensity of the test cell and that of the control cell may be an indicator of GCase activity, where an increase in fluorescence intensity in the test cell when compared to the control cell indicates GCase activity.

In some embodiments, GCase activity may be determined at different time points e.g., to monitor GCase activity. For example, the GCase activity of a test cell may be determined at a time point from 1 min-60 min, 1 h-5 h, 1 h-12 h, 1 h-24 h, 24 h-48 h, or any specific time within any of these ranges, such as 1 min, 2 min, 3 min, 4 min, 5 min, 10 min, 15 min, 20 min, 25 min, 30 min, 35 min, 40 min, 45 min, 50 min, 55 min, 60 min, 70 min, 80 min, 90 min, 100 min, 110 min, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or 48 h.

In some aspects, a compound according to the present disclosure may be useful for determining the location of GCase activity within a cell, such as within the ER, Golgi or lysosomal compartment. For example, a test cell may be contacted with a compound according to the present disclosure (e.g., a fluorescence-quenched GCase substrate) under conditions suitable for hydrolytic cleavage of the fluorescence-quenched GCase substrate by a GCase. The fluorescence intensity of the test cell may be visualized using standard techniques, such as fluorescence imaging techniques. The fluorescence intensity of the test cell may be compared to that of a control cell (e.g., a cell that has not been exposed to, or contacted with, a compound according to the present disclosure) to determine background or non-specific fluorescence. The difference between localization of the fluorescence intensity of the test cell and that of the control cell may be an indicator of GCase activity in different cell compartments, where an increase in fluorescence intensity in a particular compartment of the test cell when compared to the control cell indicates GCase activity in that cell compartment.

In some embodiments, such methods may further include determining the location of GCase protein levels within a cell, such as within the ER, Golgi, or lysosomal compartment. For example, the test cell and the control cell may be contacted with an antibody that specifically binds a GCase (the “GCase antibody”) and visualized using standard techniques. The difference between localization of the GCase antibody in the test cell and the control cell may be an indicator of GCase protein levels in different cell compartments.

In some aspects, a compound of the present disclosure may be useful for determining the effects of a GCase modulator in a cell. For example, a test cell may be contacted with a GCase modulator. The test cell and a control cell (e.g., a cell that has not been exposed to, or contacted with, the GCase modulator) may be contacted with a compound according to the present disclosure (e.g., a fluorescence-quenched GCase substrate) under conditions suitable for hydrolytic cleavage of the fluorescence-quenched GCase substrate by a GCase. The fluorescence intensity of the test cell and the control cell may be determined using standard techniques. The difference between the fluorescence intensity of the test cell and that of the control cell may be an indicator of the effect of the GCase modulator on GCase activity, where a difference in fluorescence intensity of the test cell when compared to the control cell indicates GCase modulation.

A “GCase modulator” may be any molecule that modulates the activity of a GCase. By “modulate,” “modulation” or “modulating” means an increase or decrease by any value between about 10% and about 90%, or of any value between about 30% and about 60%, or over about 100%, or an increase or decrease by about 1-fold, 2-fold, 5-fold, 10-fold or more, in comparison to a reference sample or compound, or in comparison to a wild type GCase. A “GCase modulator” may be a “GCase inhibitor” or a “GCase activator” or a “GCase chaperone” or a “GCase activity enhancer.”

A “GCase inhibitor” may be any molecule that inhibits the activity of a GCase, for example, the ability to inhibit the cleavage of glucose from glucosylceramide or the ability to inhibit the cleavage of glucose from a suitable substrate molecule such as, for example, 4-methylumbelliferone β-D glucopyranoside. By “inhibit,” “inhibition” or “inhibiting” means a decrease by any value between about 10% and about 90%, or of any value between about 30% and about 60%, or over about 100%, or a decrease by about 1-fold, 2-fold, 5-fold, 10-fold or more, in comparison to a reference sample or compound, or in comparison to a wild type GCase. It is to be understood that the inhibiting does not require full inhibition. In some embodiments, the inhibition may be transient. Examples of representative GCase inhibitors include: (3R,4R,5R)-5-(hydroxymethyl)piperidine-3,4-diol (isofagomine), and (3R,4R,5S)-5-(difluoromethyl)piperidine-3,4-diol (AT3375). For a discussion of inhibitors of GCase, see for example, Trapero et al.³⁶

A “GCase activator” may be a small molecule that enhances the enzymatic activity of a GCase by specifically binding to an allosteric site, a natural ligand binding site, or another site on a GCase. By “enhance,” “enhancement” or “enhancing” means a increase by any value between about 10% and about 90%, or of any value between about 30% and about 60%, or over about 100%, or an increase by about 1-fold, 2-fold, 5-fold, 10-fold or more, in comparison to a reference sample or compound, or in comparison to a wild type GCase. For a discussion of GCase activators see for example Patnaik et al.³⁷

A “GCase chaperone” may be a molecule that acts as a pharmacological chaperone for GCase. A pharmacological chaperone, as used herein, is a small molecule that may be useful to increase enzyme levels in a cell or cellular compartment, as in for example pharmacological chaperone therapy or “PCT”.^{10,38} In PCT, a small molecule binds to an enzyme, such as a GCase, in the endoplasmic reticulum (ER) or Golgi apparatus (Golgi) and enhances the ability of the enzyme to reach, and/or maintain, its proper fold. Compounds that are pharmacological chaperones may be active-site inhibitors, but may also bind to other sites on the enzyme such as allosteric sites, natural ligand binding sites, or other sites. Without being bound to any particular hypothesis, binding of the chaperone to the enzyme may enhance its trafficking through the secretory pathway to its proper cellular destination, to allow the enzyme to carry out its normal functions.

A “GCase activity enhancer” may be a compound that increases GCase activity within cells. A GCase activity enhancer may be a GCase chaperone. A GCase activity enhancer may be a GCase activator. A GCase activity enhancer may increase GCase activity within cells through a mechanism that is distinct from a GCase chaperone or a GCase activator.

In some aspects, a compound of the present disclosure may be useful for determining the efficacy of a GCase-directed therapy. For example, a test cell, such as a cell obtained from a subject treated with or exposed to a GCase-directed therapy, and a control cell (e.g., a cell from a subject not treated with or exposed to the GCase-directed therapy) may be contacted with a compound according to the present disclosure (e.g., a fluorescence-quenched GCase substrate) under conditions suitable for hydrolytic cleavage of the fluorescence-quenched GCase substrate by a GCase. The fluorescence intensity of the test cell and the control cell may be determined using standard techniques. The difference between the fluorescence intensity of the test cell and that of the control cell may be an indicator of the efficacy of the GCase-directed therapy. A “GCase-directed therapy” may be, without limitation, one or more of: a GCase enzyme replacement therapy (ERT), a GCase chaperone therapy, a GCase activator therapy, or a GC activity enhancer therapy.

In some embodiments, a compound of the present disclosure may be useful for determining the efficacy of a preclinical GCase-directed therapy. In alternative embodiments, a compound of the present disclosure may be useful for determining the efficacy of a clinical GCase-directed therapy. In alternative embodiments, a compound of the present disclosure may be useful for determining the efficacy of an experimental GCase-directed therapy. In some embodiments, the cells may be derived from a subject treated with a GCase-directed therapy. In some embodiments, the cells may be from tissue derived from a subject treated with a GCase-directed therapy. In some embodiments, the subject may be a human subject. In some embodiments, the subject may be a non-human subject. In some embodiments, the cells may be derived from a human subject treated with a GCase-directed therapy. In some embodiments, the cells may be fibroblasts or PBMCs. In some embodiments, the tissue may be derived from a human subject treated with a GCase-directed therapy. In some embodiments, the tissue may be a skin punch derived from a human subject treated with a GCase-directed therapy.

In some aspects, a compound of the present disclosure may be useful for screening for a GCase activity enhancer in for example a cell-based library screen. For example, a test cell may be contacted with a test compound, from, for example a compound library. The test cell and a control cell (e.g., a cell that has not been exposed to, or contacted with, a compound from the compound library) may be contacted with a compound according to the present disclosure (e.g., a fluorescence-quenched GCase substrate) under conditions suitable for hydrolytic cleavage of the fluorescence-quenched GCase substrate by a GCase. The intensity of the fluorescence emission (the “fluorescence intensity”) of the test cell and the control cell may be determined using standard techniques. The difference between the fluorescence intensity of the test cell and that of the control cell may determine whether the test compound is a GCase activity enhancer, where an increase in fluorescence intensity in the test cell when compared to the control cell indicates that the test compound is a GCase activity enhancer. In alternative embodiments, the cell-based library screen may be a high-throughput screen. In alternative embodiments, the cell-based library screen may be a phenotypic screen.

Any suitable cell (e.g., test cell and/or control cell) may be used in the methods according to the disclosure. In some embodiments, the cell may be an eukaryotic cell. In some embodiments, the cell may be a mammalian cell. In some embodiments, the cell may be a non-human cell. In some embodiments, the cell may be a human cell. In some embodiments, the cell may be an immortalized cell. In some embodiments, the cell may be a stem cell. In some embodiments, the cell may be a pluripotent stem cell. In some embodiments, the cell may be a transfected cell. In some embodiments, the cell may be an inducible transfected cell. In some embodiments, the cell may be an inducible transfected stem cell. In some embodiments, the cells may be cultured. In some embodiments, the cells may be a primary cell. In some embodiments, the cell may be derived from one of more of: primary cells, cultured cells, stem cells, pluripotent stem cells, transfected cells, inducible transfected cells, inducible transfected stem cells, human cells, non-human cells, and immortalized cells. In some embodiments, the cell may be non-human blood cells. In some embodiments, the cell may be a human blood cell. In some embodiments, the cell may be a live cell. In some embodiments, the cell may be an actively growing cell. In some embodiments, the cell may be a quiescent cell. In some embodiments, the cell may be at any phase of the cell cycle. In some embodiments, the cell may be a fixed cell. In some embodiments, one or more of the compounds according to the present disclosure may be useful for monitoring GCase activity within a cell lysate. Examples of suitable cells include, for example: fibroblasts (e.g., human fibroblasts or non-human fibroblasts), peripheral blood mononuclear cells (PBMCs), such as human or non-human PBMCs, SK-N-SH cells, SK-SY5Y cells, SH-SY5Y cells, CHO cells, HEK cells, PC12 cells, glial cells, astrocytes, neuronal cells, or LUHIMES cells. In some embodiments, the cells may be derived from a subject treated with a GCase-directed therapy.

In some embodiments, one or more of the compounds according to the present disclosure may be useful for monitoring GCase activity within a tissue. In some embodiments, the tissue may be mammalian tissue. In some embodiments, the tissue may be non-human tissue. In some embodiments, the tissue may be human tissue. In some embodiments, the tissue may be human biopsy tissue. Examples of suitable tissue include, for example: skin punch tissue, brain tissue, liver tissue, spleen tissue, kidney tissue, or any other biopsy tissue sample. In some embodiments, the cells may be from tissue derived from a subject treated with a GCase-directed therapy. In some embodiments, the tissue may be derived from a human subject treated with a GCase-directed therapy. In some embodiments, the tissue may be a skin punch derived from a human subject treated with a GCase-directed therapy. In some embodiments, the cell or tissue may be provided in a sample. A “sample” can be any organ, tissue, cell, or cell extract isolated from a subject, such as a sample isolated from an animal, such as a mammal having a condition that is modulated by a GCase. For example, a sample can include, without limitation, cells or tissue (e.g., from a biopsy or autopsy) from bone, brain, breast, colon, muscle, nerve, ovary, prostate, retina, skin, skeletal muscle, intestine, testes, heart, liver, lung, kidney, stomach, pancreas, uterus, adrenal gland, tonsil, spleen, soft tissue, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, urine, stool, saliva, placental extracts, amniotic fluid, a cryoprecipitate, a cryosupernatant, a cell lysate, mammalian cell culture or culture medium, products of fermentation, ascitic fluid, proteins present in blood cells, or any other specimen, or any extract thereof, obtained from a patient (human or animal), test subject, or experimental animal. A sample may also include, without limitation, products produced in cell culture by normal or transformed cells (e.g., via recombinant DNA or monoclonal antibody technology). A sample may also include, without limitation, any organ, tissue, cell, or cell extract isolated from a non-mammalian subject, such as an insect or a worm. A “sample” may also be a cell or cell line created under experimental conditions, that is not directly isolated from a subject. A sample can also be cell-free, artificially derived or synthesized. A “control” may include a sample obtained for use in determining base-line expression or activity. A control may also include a previously established standard. Accordingly, any test or assay conducted according to the disclosure may be compared with the established standard and it may not be necessary to obtain a control sample for comparison each time. As used herein, a subject may be a human, non-human primate, rat, mouse, cow, horse, pig, sheep, goat, dog, cat, etc. The subject may be a clinical patient, a clinical trial volunteer, an experimental animal, etc. The subject may be suspected of having or at risk for having a condition that is modulated by a GCase, be diagnosed with a condition that is modulated by GCase, or be a control subject that is confirmed to not have a condition that is modulated by a GCase. Diagnostic methods for conditions modulated by a GCase, and the clinical delineation of such diagnoses, are known to those of ordinary skill in the art.

Suitable techniques to measure fluorescence intensity include, for example, fluorescence microscopy, confocal microscopy, use of a fluorescent plate reader, high content imaging, photoacoustic imaging, ratiometric imaging, flow cytometry, and fluorescence-activated cell sorting (FACS). Suitable techniques to measure fluorescence intensity with tissues include, for example, fluorescence microscopy, confocal microscopy, use of a fluorescent plate reader, high content imaging, photoacoustic imaging, and ratiometric imaging.

As will be appreciated by a person skilled in the art, the methods described herein for example for monitoring GCase activity within cells or tissue, or visualizing localization of GCase activity within lysosomes, may also be represented, for example, as in Scheme H:

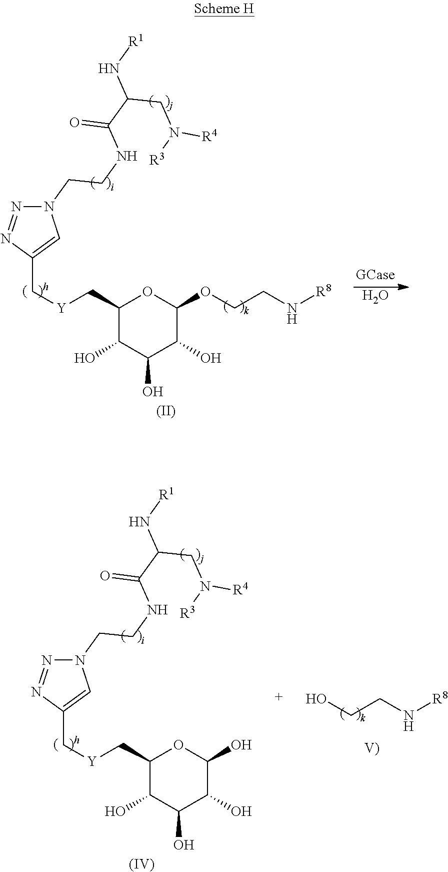

where R¹ may be a suitable fluorophore and R⁸ may be a suitable quencher; or R¹ may be a suitable quencher and R⁸ may be a suitable fluorophore; R¹ may be (5,5-difluoro-7,9-dimethyl-dipyrrolo[1,2-c:2′,1′-f][1,3,2]diazaborinin-4-ium-5-uid-3-yl)propanoyl and R⁸ may be 4-((4-((E)-(2-methoxy-5-methyl-4-((E)-(4-methyl-2-nitrophenyl)diazenyl)phenyl)diazenyl)phenyl)(methyl)amino)butanoyl; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; h may be an integer from 1 to 5; i may be an integer from 1 to 5; j may be a n integer from 1 to 5; k may be an integer from 1 to 10; Y may be an O or S or NH or CH₂. In this aspect, cells or tissue are treated with the fluorescence quenched substrate of Formula (II) (which is not fluorescent, due to internal quenching), and a GCase enzyme hydrolytically cleaves the substrate of Formula (I) to generate the sugar of Formula (IV) and the alcohol of Formula (V). When R¹ is a suitable fluorophore, the sugar of Formula (IV) will be fluorescent, as the fluorophore R¹ is no longer internally quenched by the quencher R⁸. When R⁸ is a suitable fluorophore, the alcohol of Formula (V) will be fluorescent, as the fluorophore R⁸ is no longer internally quenched by the quencher R¹. Measuring the fluorescence intensity due to the compound of Formula (IV) or the compound of Formula (V) thus provides a method for monitoring GCase activity within cells or tissue.

As will be appreciated by a person skilled in the art, the methods described herein for example for assessment of GCase inhibition in cells or tissue may also be represented, for example, as in Scheme I:

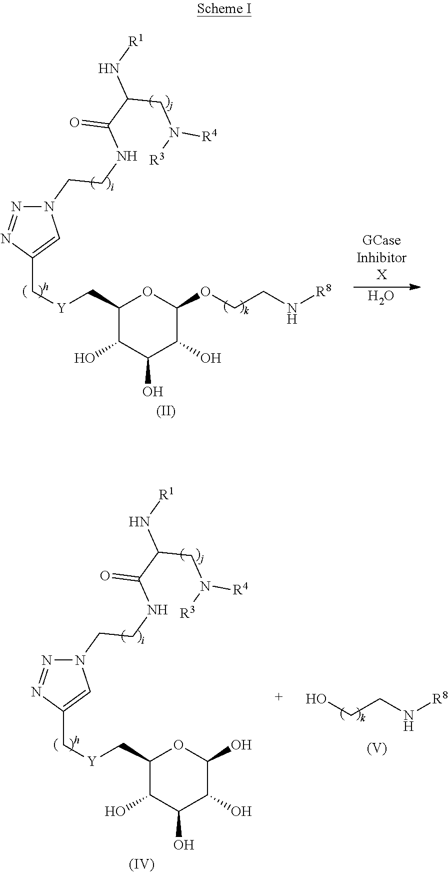

where R¹ may be a suitable fluorophore and R⁸ may be a suitable quencher; or R¹ may be a suitable quencher and R⁸ may be a suitable fluorophore; R¹ may be (5,5-difluoro-7,9-dimethyl-dipyrrolo[1,2-c:2′,1′-f][1,3,2]diazaborinin-4-ium-5-uid-3-yl)propanoyl and R² may be 4-((4-((E)-(2-methoxy-5-methyl-4-((E)-(4-methyl-2-nitrophenyl)diazenyl)phenyl)diazenyl)phenyl)(methyl)amino)butanoyl; R³ and R⁴ may be the same or different and selected from a list of hydrogen, or an alkyl chain of one to 4 carbons, or a peptide of between 1 and 10 amino acids in length that is covalently linked using an amide bond, or another suitable bioconjugation reaction, or using a suitable click reaction; h may be an integer from 1 to 5; i may be an integer from 1 to 5; j may be a n integer from 1 to 5; k may be an integer from 1 to 10; Y may be an O or S or NH or CH₂. In this aspect, cells or tissue are treated with a GCase inhibitor and the fluorescence quenched substrate of Formula (II), and the GCase inhibitor prevents the GCase enzyme from hydrolytically cleaving the substrate of Formula (II) to generate either the fluorescent sugar of Formula (IV) or the fluorescent alcohol of Formula (V). Carrying out this procedure using, for example, varying concentrations of a GCase inhibitor and measuring the fluorescence intensity due to either the compound of Formula (IV) or the compound of Formula (V) thus gives a measurement of the extent to which a GCase enzyme is blocked by a GCase inhibitor, and provides a method for assessment of GCase inhibition in cells or tissue.

As will be appreciated by a person skilled in the art, the methods described herein for example for monitoring GCase activity within cells or tissue, or visualizing localization of GCase activity within lysosomes, may also be represented, for example, as in Scheme H:

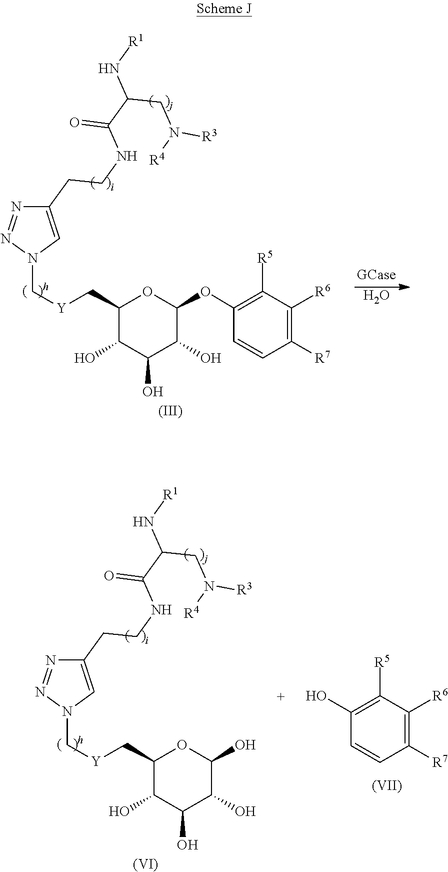

where R¹ may be a suitable fluorophore or a suitable quencher; R¹ may 5-carboxy-2-(6-(dimethylamino)-3-(dimethyliminio)-3H-xanthen-9-yl)benzoyl and R⁵, R⁶ and R⁷ may be 4-((4-((E)-(2-methoxy-5-methyl-4-((E)-(4-methyl-2-nitrophenyl)diazenyl)phenyl)diazenyl)phenyl)(methyl)amino)butanoyl; R³ and R⁴ may be the same or different and selected from a list of hydrogen, or an alkyl chain of one to 4 carbons, R⁵,R⁶ and R⁷ may be the same or different and selected from a list of hydrogen, fluorine, NO₂ group, or a suitable quencher or a suitable fluorophore; h may be an integer from 1 to 5; i may be an integer from 1 to 5; j may be a n integer from 1 to 5; Y may be an O or S NH or CH₂. In this aspect, cells or tissue are treated with the fluorescence quenched substrate of Formula (III) (which is not fluorescent, due to internal quenching), and a GCase enzyme hydrolytically cleaves the substrate of Formula (III) to generate the sugar of Formula (VI) and the alcohol of Formula (VII). When R¹ is a suitable fluorophore, the sugar of Formula (VI) will be fluorescent, as the fluorophore R¹ is no longer internally quenched by the quencher at R⁵, R⁶ and R⁷. When R⁵,R⁶ and R⁷ is a suitable fluorophore, the alcohol of Formula (VII) will be fluorescent, as the fluorophore R⁵, R⁶ and R⁷ is no longer internally quenched by the quencher R¹. Measuring the fluorescence intensity due to the compound of Formula (VI) or the compound of Formula (VII) thus provides a method for monitoring GCase activity within cells or tissue.

As will be appreciated by a person skilled in the art, the methods described herein for example for assessment of GCase inhibition in cells or tissue may also be represented, for example, as in Scheme I:

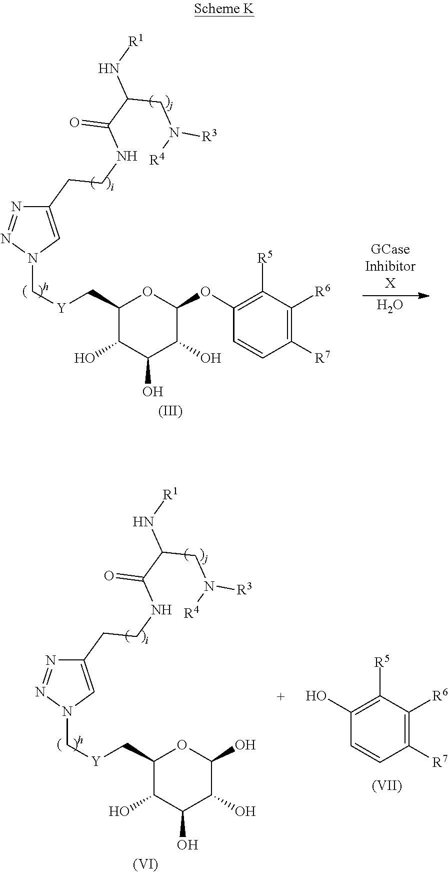

where R¹ may be a suitable fluorophore or a suitable quencher; R¹ may 5-carboxy-2-(6-(dimethylamino)-3-(dimethyliminio)-3H-xanthen-9-yl)benzoyl and R⁵,R⁶ and R⁷ may be 4-((4-((E)-(2-methoxy-5-methyl-4-((E)-(4-methyl-2-nitrophenyl)diazenyl)phenyl)diazenyl)phenyl)(methyl)amino)butanoyl; R³ and R⁴ may be the same or different and selected from a list of hydrogen, or an alkyl chain of one to 4 carbons, R⁵,R⁶ and R⁷ may be the same or different and selected from a list of hydrogen, fluorine, NO₂ group, or a suitable quencher or a suitable fluorophore; h may be an integer from 1 to 5; i may be an integer from 1 to 5; j may be a n integer from 1 to 5; Y may be an O or S NH or CH₂. In this aspect, cells or tissue are treated with a GCase inhibitor and the fluorescence quenched substrate of Formula (III), and the GCase inhibitor prevents the GCase enzyme from hydrolytically cleaving the substrate of Formula (III) to generate either the fluorescent sugar of Formula (VI) or the fluorescent alcohol of Formula (VII). Carrying out this procedure using, for example, varying concentrations of a GCase inhibitor and measuring the fluorescence intensity due to either the compound of Formula (VI) or the compound of Formula (VII) thus gives a measurement of the extent to which a GCase enzyme is blocked by a GCase inhibitor, and provides a method for assessment of GCase inhibition in cells or tissue.

A suitable concentration for use of a compound according to the disclosure, such as a compound of Formula (I), (II), (IIa)-(IIl), (III), (IIIa)-(IIIe), or an acceptable salt thereof, to monitor GCase activity within cells or tissue may be any integer from 0.1 nM-0.1 M, 0.1 nM-0.05 M, 0.05 nM-15 μM, 0.01 nM-100 μM, or 1-500 μM, or any specific concentration within any of these ranges, such as 10 nM, 20 nM, 30 nM, 40 nM, 50 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, or 500 μM.

A suitable incubation time for use of a compound according to the disclosure, such as a compound of Formula (I), (II), (IIa)-(IIl), (III), (IIIa)-(IIIe), or an acceptable salt thereof, to monitor GCase activity within cells or tissue may be any integer from 5 min-60 min, 1 h-5 h, 1 h-12 h, 1 h-24 h, 24 h-48 h, 1 day-2 days, 1 day-5 days, 1 day-7 days, 1 day-14 days, 1 day-28 days, or any specific time within any of these ranges, such as 5 min, 10 min, 15 min, 20 min, 25 min, 30 min, 60 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 1.5 days, 2 days, 2.5 days, 3 days, 3.5 days, 4 days, 4.5 days, 5 days, 6 days, 7 days, 8 days, 9 days, 10 days, 11 days, 12 days, 13 days, or 14 days.

As used herein the singular forms “a”, “and”, and “the” include plural referents unless the context clearly dictates otherwise. For example, “a compound” refers to one or more of such compounds, “a cell” refers to a plurality of cells, while “the enzyme” includes a particular enzyme as well as other family member equivalents thereof as known to those skilled in the art.

Various alternative embodiments and examples of the invention are described herein. These embodiments and examples are illustrative and should not be construed as limiting the scope of the invention.

### Abbreviations

AT3375=(3R,4R,5S)-5-(difluoromethyl)piperidine-3,4-diol
BHQ®1-acid=BHQ®2-NHS=2,5-dioxopyrrolidin-1-yl 4-((4-((E)-(2,5-dimethoxy-4-((E)-(4-nitrophenyl)diazenyl)phenyl)diazenyl)phenyl)(methyl)amino)butanoate BODIPY®FL-acid=3-(5,5-difluoro-7,9-dimethyl-dipyrrolo[1,2-c:2′,1′-f][1,3,2]diazaborinin-4-ium-5-uid-3-yl)propanoic acid
DAPI=4′,6-diamidino-2-phenylindole
DCM=dichloromethane
DIPEA=diisopropylethylamine
DMEM=Dulbecco's Modified Eagle's medium
DMF=N,N-dimethylformamide
DMSO=dimethylsulfoxide
EtOAc=ethyl acetate
FBS=fetal bovine serum
HBTU=2-(1H-benzotriazol-1-yl)-1,1,3,3-tetramethyluronium hexafluorophosphate
HEPES=4-(2-hydroxyethyl)-1-piperazineethanesulfonic acid
MEM=minimum essential media
MeOH=methanol
PBS=phosphate buffered saline

### General Procedures and Conditions

### General Information

¹H and ¹³C NMR spectra were obtained using Bruker AVII 600 (600 MHz for ¹H and 151 MHz for ¹³C) and Bruker AVIII 400 (400 MHz for ¹H and 101 MHz for ¹³C) spectrometers. Unless stated otherwise, deuterated methanol (CD₃OD) was used as the solvent with CD₂HOD (δH 3.31) or CD₃OD (δC 49.00) being employed as internal standards. Abbreviations used to describe the observed peaks: s, singlet; d, doublet; t, triplet; q, quadruplet; m, multiplet and bs, broad singlet. HPLC purification was performed on an Agilent 1100 series instrument with an Eclipse XDB C18 column (5.0 m, 9.4×250 mm) or a Zorbax 300SB-C8 column (5.0 m, 9.4×250 mm) using HPLC grade solvents. High resolution mass spectrometry (HRMS) analysis was performed using a Bruker maXis ToF LC/MS/MS instrument using positive or negative electrospray ionization (ESI). Flash chromatography was performed either under positive pressure with Fisher Scientific silica gel (230-400 mesh) or using RediSep® Gold normal phase columns (4 g, 24 g, or 40 g) on a Combiflash® RF+ instrument. Thin-layer chromatography (TLC) was performed using Merck silica gel 60 F254 aluminum-backed plates that were stained by heating (>200° C.) with 5% sulfuric acid in EtOH or with a solution of phosphomolybdic acid (2.5% w/v), cerium sulfate (1% w/v), and sulfuric acid (6% v/v) in water. Percentage yields for chemical reactions are quoted only for those compounds that were purified by recrystallization or by column chromatography, and for which the purity was assessed verified by ¹H NMR spectroscopy. Chemicals and solvents were obtained from Sigma-Aldrich and used without further purification unless otherwise noted. LysoTracker® Red (DND-99), and alpha MEM (without nucleosides) were from Life technologies. Eagle's Minimum Essential Medium (EMEM) was purchased from ATCC. Fetal Bovine Serum (FBS), Glutamax, Penicillin/Streptomycin (Pen/Strep), Trypsin-EDTA (0.25%) and Phosphate Buffer Saline (PBS) were acquired from Gibco. PE/Cy7 anti-human CD14 antibody (clone 63D3) was bought from Biolegend. AT3375 was synthesized in house and dissolved as a 20 mM stock in DMSO 100%. The different substrates were kept as dry aliquots at −20° C. and freshly re-suspended in Washing Media (20 mM HEPES containing 140 mM NaCl, 2.5 mM KCl, 1.8 mM CaCl₂), 1 mM MgCl₂, 1 g·L⁻¹ D-Glucose, and Pen/Strep).

### Cell Culture

SK-N-SH cells were cultured, except where indicated otherwise, in EMEM supplemented with 10% FBS, Glutamax, and 1% penicillin-streptomycin at 37° C. and 5% CO₂ at a density of 10,000 cells/mL. The media was replaced every 3-4 days and the cells passaged after reaching 70-80% confluency, which occurred approximately every 7 days.

### Live Cell Imaging

High content imaging was performed using a Wide-Field ImageXpress Micro XLS (Molecular Devices) system. All fluorescence images were processed using MetaXpress 6 software (Molecular Devices) and the Multiwavelength Cell Scoring application module. For the time-course incubation of the substrate in SK-N-SH, 3000 cells/well were seeded into a 384-well plate (Corning 4681) in a final volume of 50 μL and allowed to adhere overnight at 37° C., 5% CO₂. The cells were then treated with 5 μL of either AT3375 (final 10 μM in DMSO 1%) or vehicle (final DMSO 1%) and incubated overnight at 37° C., 5% CO₂. On the designated times before the stop, 5 μL of substrate 1 were added to reach a final concentration of 10 μM. The assay was stopped by washing 3 times with 60 μL of Washing Media (20 mM HEPES containing 140 mM NaCl, 2.5 mM KCl, 1.8 mM CaCl₂), 2H₂O, 1 mM MgCl₂, 6 H₂O, 1 g·L⁻¹ D-Glucose and 1% Pen/STREP) on an EL406 plate washer (Biotek) and adding 50 μL of Reading Media (Washing Media containing 10% FBS, AT3375 10 μM and 1 μg mL⁻¹ Hoechst 33342). After stopping the assays, the plates were incubated for 30 min at 37° C., 5% CO₂ inside the Wide-Field ImageXpress Micro XLS (Molecular Devices) system. Four fields of images were acquired at pre-defined regions of each well using a 40× Extra Long Working Distance Air objective (Nikon) and both the DAPI and FITC filter cubes (Semrock). The number of cell nuclei were determined by analysis of the DAPI channel. Fluorescence intensities above a background threshold were integrated on each image in the FITC channel for substrate 1 or the TRITC channel for substrate 2-4. The signal in each well was defined by summing up the integrated intensities on all sites and normalizing by the total number of cells on the entire well.

For the time-course incubation of the substrate in SK-N-SH, 3000 cells/well were seeded into a 384-well plate (Corning 4681) in a final volume of 50 μL and allowed to adhere overnight at 37° C., 5% CO₂. The cells were then treated with 5 μL of either AT3375 (final 10 μM in DMSO 1%) or vehicle (final DMSO 1%) and incubated overnight at 37° C., 5% CO₂. On the designated times before the stop, 5 μL of substrate were added to reach a final concentration of 10 μM. The assay was stopped by washing 3 times with 60 μL of Washing Media on an EL406 plate washer (Biotek) and adding 50 μL of Reading Media (Washing Media containing 10% FBS, 10 μM AT3375 and 1 μg mL⁻¹ Hoechst 33342). Substrate dose-response studies were carried out as previously described except that the substrate was incubated for 2 h at the specified concentrations before stopping the assay. AT3375 dose-response studies were performed as described before but AT3375 was incubated for 3 days at the specified concentrations and the substrate was incubated at 10 μM for 2 h before stopping the assay. After stopping the assays, the plates were incubated for 30 min at 37° C., 5% CO₂ inside the Wide-Field ImageXpress Micro XLS (Molecular Devices). Four fields of images were acquired at predefined regions of each well using a 40× Extra Long Working Distance Air objective (Nikon) and both the DAPI and FITC filter cubes (Semrock). Images were processed using the MetaXpress 6 software (Molecular Devices) and the Multiwavelength Cell Scoring application module. The number of cell nuclei were determined by analysis of the DAPI channel. Depending on the substrate, the fluorescence intensities above a background threshold were integrated on each image in the FITC (MDFL1 and substrate 1) channel. The signal in each well was defined by summing up the integrated intensities on all sites and normalizing by the total number of cells on the entire well.

Substrate dose-response studies were carried out as previously described except that the substrate was incubated for 2 h at the specified concentrations before stopping the assay. AT3375 dose-response studies were performed as described before but AT3375 was incubated for 3 days at the specified concentrations and the substrate was incubated at 10 μM for 2 h before stopping the assay.

AT3375 dose-response studies were performed as previously described but AT3375 was incubated for 3 days at the specified concentrations and the substrate was incubated at 10 μM for 2 h before stopping the assay.

Comparative assays between were performed as previously described but cells were treated with either substrate at 10 μM or either substrate at 10 μM with AT3375 10 μM for 2 h before stopping the assay.

### Flow Cytometry Assay of GCase Activity in SK-N-SH Cells

For the time-course incubation of the substrate in SK-N-SH, 200 000 cells/well were seeded into a 24-well plate in a final volume of 290 μL and allowed to adhere overnight at 37° C., 5% CO₂. The cells were then treated with 5 μL of either AT3375 (final 10 μM in DMSO 1%) or vehicle (final DMSO 1%) and incubated for 2 h. On the designated times before the stop, 5 μL of substrate were added to reach a final concentration of 10 μM. The assay was stopped by washing 3 times with 500 μL of Washing Media. The cells were detached by adding 250 μL of Trypsin-EDTA (0.25%, Gibco) and incubating them for 3 min at 37° C., 5% CO₂. Then 250 μL of Stop Media was added, the cells were transferred into a microtube and centrifugated for 5 min at 300 g. The supernatant was discarded, and the cell pellet was re-suspended in Stop Media to achieve a cell suspension concentration of 1×10⁶ cells mL⁻¹. Finally, the cells were strained, and the samples were kept on ice until analysis. The same protocol was used for the substrate dose-response except that the substrate was incubated at different concentrations for 1.5 h. Finally, the AT3375 dose-response in SK-N-SH was assessed using the same assay but seeding 75,000 cells/well, incubating the inhibitor/vehicle for 3 days and incubating the substrate at 10 μM for 1.5 h before stopping the assay.

### Fibroblast Cell Culture

Fibroblast cells, WT (GM0049) and L444P/P415R (GM01260) were cultured in EMEM supplemented with 15% FBS, Glutamax, and 1% penicillin-streptomycin at 37° C. and 5% CO₂ at a density of 10,000 cells/mL. The media was replaced every 3-4 days and the cells passaged after reaching 70-80% confluency, which occurred approximately every two weeks.

For the measurements of residual activity 2000 cells/well were seeded into a 384-well plate (Corning 4681) in a final volume of 50 μL and allowed to adhere overnight at 37° C., 5% CO₂. The cells were then treated with 5 μL of either AT3375 (final 10 μM in DMSO 1%) or vehicle (final DMSO 1%) and incubated for 3 days at 37° C., 5% CO₂. After inhibitor incubation L of substrate were added to reach a final concentration of 5 μM and the cells were incubated for 1 h. Finally, the assay was stopped by aspirating the media and adding 50 μL of Reading Media on the plate washer. After stopping the assays, the plates were incubated for 30 min at 37° C., 5% CO₂ inside the Wide-Field ImageXpress Micro XLS (Molecular Devices). Four fields of images were acquired at predefined regions of each well using a 40× Extra Long Working Distance Air objective (Nikon) and both the DAPI and FITC filter cubes (Semrock). Images were processed using the MetaXpress 6 software (Molecular Devices) and the Multiwavelength Cell Scoring application module. The number of cell nuclei were determined by analysis of the DAPI channel. Depending on the substrate, the fluorescence intensities above a background threshold were integrated on each image in the FITC (substrate 1) channel. The signal in each well was defined by summing up the integrated intensities on all sites and normalizing by the total number of cells on the entire well.

### Co-Localization of Substrate 1 and the Lysosomal Marker LysoTracker Red DND-99

SK-N-SH cells were seeded in 35 mm MatTek glass bottom dishes at a density of 100,000 cells/2 mL. After 2-3 days (˜50% confluency) cells were washed three time with washing media and substrate 1 was added into dishes at 10 μM in 2 mL imaging medium for 2 h. After substrate incubation, cells were washed again with imaging medium three times, and subsequently incubated for 15 min in imaging medium containing 1:10000 Hoechst (Life Technology) and 1:20000 LysoTracker Red DND99 (Life Technology). All images were acquired by a Nikon AiR confocal system with Nikon Eclipse Ti inverted microscope using a plan Apo 60× oil objectives (NA1.4). DAPI, FITC and Texas Red channels were used for Hoechst, substrate 1, and LysoTracker, respectively. For quantification of the colocalization, FIJI (ImageJ v.1.53) with co-localization plugin was used, Pearson's correlation coefficient (PCC, r value) between LysoTracker and substrate 1, were measured using three ROIs per image. To verify that no random colocalization was measured, the Costes test was also performed, which randomizes one image by moving PSF sized chunks of the image and analyze the Pearson's correlation coefficient (r value) between the randomized image and the original image.

Human Induced Pluripotent Stem Cell (iPSC) Lines

Human iPSC lines PGPC1 and PGPC17 derived from healthy individuals with whole-genome sequencing-based annotation are generous gifts from Dr. James Ellis at the Hospital for Sick Children, Toronto, Canada. Lines UOXFi001-B and UOXFi003-A obtained from EBiSC were derived from patients of Parkinson Disease (PD) carrying the heterozygous mutation GBA^{N370S/wt}. The Gaucher Disease (GD) iPSC line C43-1260 (GBA^{L444P/P415R}) line was generated from a GD patient's skin fibroblast line (GM01260, Coriell) and characterized using a contract service provided by the Tissue and Disease Modelling Core (TDMC) at the BC Children's Hospital Research Institute using their standard protocols. The genome integrity of this line was confirmed by G-banded karyotyping done at WiCell. The pluripotency of C43-1260 was tested by in vitro differentiation to the three germ layers using the STEMdiff™ Trilineage Differentiation Kit (STEMCELL Technologies). All iPSCs were cultured and expanded in mTeSR™ Plus medium (STEMCELL) according to the manual.

Live-Cell GCase Assay on iPSC-Derived Neural Progenitor Cells (NPCs)

iPSC lines including PGPC1, PGPC17, C43-1260, UOXFi001-B and UOXFi003-A were differentiated to NPCs using the STEMdiff™ Neural Induction Medium+SMADi (STEMCELL) following the monolayer-based differentiation protocol as described in the technical manual (STEMCELL). Generated NPCs were either used directly or cryopreserved in STEMdiff™ Neural Progenitor Freezing Medium (STEMCELL) and stored in a liquid nitrogen Dewar until use. Frozen NPCs were thawed and recovered in STEMdiff™ Neural Progenitor Medium (STEMCELL) for at least one passage before plating in the 384-well high-content imaging plates (Corning 4681) for the live-cell GCase assay. NPCs were prepared as single-cell suspension, counted, and seeded in the plate at the density of 70,000 cells/cm². Two days post seeding, a full medium exchange was carried out. Next day, the live-cell GCase assay was initiated by incubating NPCs with GCase activity probe substrate 1 at designated concentrations for designated lengths of time. The assay was then stopped by the addition of the specific GCase inhibitor AT3375 to final 10 μM, and Hoechst 33342 was added to final 1 μg/ml at the same time for staining the nuclei. After the incubation with AT3375 and Hoechst for 30 mins, NPCs were imaged using a high content widefield microscope (ImageXpress XLS, Molecular Devices). Micrographs will be processed using high-content imaging analysis software MetaXpress (Molecular Devices) to yield the mean FTIC intensity level (representing the GCase activity) per cell for each well.

Live-Cell GCase Assay on iPSC-Derived Monocytes

The five iPSC lines PGPC1, PGPC17, C43-1260, UOXFi001-B and UOXFi003-A were differentiated to monocytes using the STEMdiff™ Monocyte Kit (STEMCELL) following the differentiation protocol as described in the technical manual (STEMCELL). Monocytes were harvested on day 19 and 23 of the differentiation process and incubated with probe substrate 1 at designated concentrations for designated lengths of time. The assay was then stopped by the addition of specific GCase inhibitor AT3375 to final 10 μM followed by staining with anti-CD14 antibody (Biolegend, Cat. #367112). After staining dead cells with 7-AAD or DAPI, monocyte samples were analyzed using the BD LSRFortessa™ flow cytometer. Flow data were analyzed using software FlowJo. Dead cells were first removed by gating on 7-AAD or DAPI signal intensity, and then monocytes were then gated out using the CD14 signal intensity.

### Cellular Lysate GCase Activity Assay

Cells were lysed in 100 μL of M-PER mammalian extraction buffer containing 20 mg/mL protease inhibitor mixture. Cell lysates were then sonicated at 10% amplitude for 8 seconds and centrifuged at 13000 rpm for 20 minutes at 4° C. Supernatant was calculated and the protein concentration was measured using a nanodrop where 1 Abs=1 mg/mL. Protein concentration was adjusted to 4 mg/mL in M-PER mammalian extraction buffer containing 20 mg/mL protease inhibitor mixture. The reactions were prepared in GBA1-based buffer (0.1 M citric acid, 0.2 M Na₂HPO₄, pH 5.2) with 0.1% Triton X-100 and 0.25% sodium taurodeoxycholate. Equal amounts of lysate from each cell line were used, and the final assay contained 0.1 mg/mL cell lysate. The lysate was preincubated with either 10 μM of Miglustat to control for GBA2 activity or 10 μM of AT3375 and 10 μM Miglustat to control for GBA2 activity and non-specific for 5 minutes at 37° C. Following pre-incubation, 4MU-Glc was added to each well to final 2 mM to initiate the reaction and allowed to proceed for 20 minutes at 37° C. The reaction was finally stopped with equal volume of stopping buffer containing 0.5 M NaOH, 0.3 M glycine, pH 10.5 and fluorescence was measured at 365/450 nm using a plate reader with bandwidth of 9 nm and read height of 6 mm at a gain of 100. Value of GCase activity was standardized by subtracting the value yielded from assay condition with GCase+GBA2 inhibitors. Standardized GCase activity value from each line was then normalized by dividing that of PGPC1, while activity of PGPC1 was arbitrarily set as 1.

### Example 1

3-Azidopropylamine (287 mg; 2.92 mmol) was added to a solution of N(α)-Boc-N(ε),N(ε)-dimethyl-L-lysine (400 mg; 1.46 mmol) in DMF (15 mL) at room temperature. Subsequently, diisopropylethylamine (761 μL; 4.37 mmol) and HBTU (609 mg; 1.61 mmol) were added to the reaction flask and the mixture was left to stir overnight at room temperature. TLC: R_{f} 0.15 in 9% MeOH in CHCl₃ Once the reaction was complete, as judged by TLC, the solution was concentrated, and the crude product was purified using silica gel column chromatography. The desired product was enriched by eluting with a gradient from 5% MeOH in CHCl₃ to 9% MeOH in CHCl₃ with 1% triethylamine. The resulting crude product was dissolved in DCM (30 mL) and washed with aqueous NaOH (5% w/v, 10 mL), the organic layer was separated, dried over sodium sulfate, and concentrated to give pure tert-butyl (S)-(1-((3-azidopropyl)amino)-6-(dimethylamino)-1-oxohexan-2-yl)carbamate. Yield: 75%; 390.6 mg. HRMS MS: [M+H]⁺ calcd 357.2609, found 357.2614. ¹H NMR (600 MHz, Chloroform-d) δ 6.81 (s, 1H), 5.42 (d, J=7.9 Hz, 1H), 4.02 (d, J=8.4 Hz, 1H), 3.34 (dt, J=16.1, 6.6 Hz, 4H), 2.33-2.21 (m, 2H), 2.21 (d, J=1.5 Hz, 6H), 1.86-1.75 (m, 3H), 1.62 (dq, J=14.8, 7.7 Hz, 1H), 1.51 (ddt, J=22.2, 14.4, 6.7 Hz, 2H), 1.44 (s, 9H), 1.38 (m, J=7.6 Hz, 2H). ¹³C NMR (151 MHz, CDCl₃) δ 172.42, 155.87, 79.92, 59.05, 54.61, 49.12, 45.33, 36.85, 31.93, 28.77, 28.33, 26.99, 23.15.

Tert-butyl (S)-(1-((3-azidopropyl)amino)-6-(dimethylamino)-1-oxohexan-2-yl)carbamate (390.4 mg; 1.10 mmol) was added to a round bottom which had been cooled to 0° C. using an ice bath. Trifluoroacetic acid (11 mL), cooled to 0° C., was added to the round bottom flask in a dropwise fashion. The reaction was stirred for 15 minutes at 0° C. after which the solvent was removed under reduced pressure. The resulting residue was washed with cold diethyl ether (20 mL) to give pure (S)-2-amino-N-(3-azidopropyl)-6-(dimethylamino)hexanamide. Yield: 100%; 529.5 mg HRMS MS: [M+H]⁺ calc'd 257.2084, found 257.2087. ¹H NMR (600 MHz, Methanol-d₄) δ 3.86 (t, J=6.6 Hz, 1H), 3.41 (t, J=6.7 Hz, 2H), 3.37-3.34 (m, 2H), 3.18-3.12 (m, 2H), 2.90 (s, 6H), 1.99-1.75 (m, 6H), 1.51-1.40 (m, 1H). ¹³C NMR (151 MHz, MeOD) δ 168.61, 56.98, 52.77, 48.59, 41.98, 36.59, 30.62, 28.17, 23.72, 21.50.

To a solution of BODIPY® Fl-acid(26.9 mg; 0.092 mmol) in DMF (1 mL) was added diisopropylethylamine (66 μL; 0.378 mmol) and 2-(1H-benzotriazol-1-yl)-1,1,3,3-tetramethyluronium hexafluorophosphate (38.3 mg; 0.101 mmol). After stirring the reaction mixture for 20 minutes a solution of (S)-2-amino-N-(3-azidopropyl)-6-(dimethylamino)hexanamide (40.4 mg; 0.84 mmol) in DMF (1 mL) was added dropwise and the resulting solution was let stir at room temperature for another 30 minutes. TLC: R_{f} 0.1 in 9% MeOH in CHCl₃. After reaction completion as judged by TLC, the mixture was concentrated, and the crude product was purified using silica gel column chromatography. Crude product was eluted with a gradient of 5% MeOH in CHCl₃ to 10% MeOH in CHCl₃. The resulting crude product was then dissolved in DCM (15 mL) and washed with 5% aqueous sodium hydroxide (5 mL) to give pure (S)—N-(3-azidopropyl)-2-(3-(5,5-difluoro-7,9-dimethyl-dipyrrolo[1,2-c:2′,1′-f][1,3,2]diazaborinin-4-ium-5-uid-3-yl)propanamido)-6-(dimethylamino)hexanamide. Yield: 88%; 39.1 mg HRMS MS: [M+H]⁺ calc'd 531.3178, found 531.3179. ¹H NMR (600 MHz, Chloroform-d) δ 7.13 (s, 1H), 6.98-6.71 (m, 2H), 6.45 (d, J=7.8 Hz, 1H), 6.30 (d, J=4.0 Hz, 1H), 6.16 (s, 1H), 4.37 (q, J=7.8 Hz, 1H), 3.31 (dq, J=12.5, 6.3 Hz, 7H), 2.75-2.69 (m, 2H), 2.67-2.56 (m, 5H), 2.49 (s, 6H), 2.28 (s, 3H), 1.92-1.73 (m, 3H), 1.72-1.56 (m, 3H), 1.43-1.30 (m, 3H).¹³C NMR (151 MHz, CDCl₃) δ 172.03, 171.69, 160.72, 156.76, 144.22, 135.30, 133.31, 128.03, 123.85, 120.64, 117.03, 58.23, 52.74, 49.15, 44.19, 36.91, 35.47, 31.40, 28.73, 25.35, 24.69, 22.43, 15.00, 11.34.

To a solution of BHQ®1-carboxylic acid (329.1 mg; 0.625 mmol) and diisopropylethyl amine (201.7 mg; 1.56 mmol) in anhydrous DMF (4.25 mL) was added HBTU (237.0 mg; 0.625 mmol). After stirring for 30 minutes, the reaction mixture was added dropwise to a solution of (2R,3R,4S,5S,6R)-2-(3-aminopropoxy)-6-((prop-2-yn-1-yloxy)methyl)tetrahydro-2H-pyran-3,4,5-triol³⁵ (143.4 mg; 0.521 mmol) in DMF (2 mL). The resulting solution was stirred in the dark for 3 hours. TLC: R_{f} 0.3 in 9% MeOH in CHCl₃. After reaction completion as judged by TLC, the mixture was concentrated, and the crude compound was purified using silica gel column chromatography using a gradient of 5% MeOH in CHCl₃ to 10% MeOH in CHCl₃ yielding pure 4-((4-((E)-(2-methoxy-5-methyl-4-((E)-(4-methyl-2-nitrophenyl)diazenyl)phenyl)diazenyl)phenyl)(methyl)amino)-N-(3-(((2R,3R,4S,5S,6R)-3,4,5-trihydroxy-6-((prop-2-yn-1-yloxy)methyl)tetrahydro-2H-pyran-2-yl)oxy)propyl)butanamide Yield: 67%; 271.8 mg. HRMS MS: [M+H]⁺ calc'd 762.3457, found 762.3467. ¹H NMR (600 MHz, DMSO-d₆) δ 7.95 (s, 1H), 7.84-7.78 (m, 3H), 7.77 (d, J=8.2 Hz, 1H), 7.71-7.67 (m, 1H), 7.52 (s, 1H), 7.29 (s, 1H), 6.88 (d, J=9.3 Hz, 2H), 5.11-5.03 (m, 2H), 5.00 (d, J=5.0 Hz, 1H), 4.31-4.09 (m, 3H), 3.93 (s, 3H), 3.83-3.69 (m, 2H), 3.47 (dt, J=8.9, 6.6 Hz, 4H), 3.42 (t, J=2.4 Hz, 1H), 3.28 (ddd, J=9.8, 6.5, 1.8 Hz, 1H), 3.21-3.09 (m, 3H), 3.07 (s, 3H), 3.05-2.98 (m, 1H), 2.96 (ddd, J=9.0, 7.8, 4.8 Hz, 1H), 2.64 (s, 3H), 2.50 (s, 3H), 2.16 (t, J=7.3 Hz, 2H), 1.80 (p, J=7.4 Hz, 2H), 1.68 (p, J=6.7 Hz, 2H). ¹³C NMR (151 MHz, DMSO) δ 171.97, 154.80, 152.34, 150.55, 146.85, 145.18, 143.98, 143.04, 142.76, 134.37, 132.73, 125.98, 124.74, 120.55, 118.95, 111.89, 103.19, 99.61, 80.94, 77.52, 77.05, 75.66, 73.81, 70.51, 69.69, 67.18, 58.29, 56.40, 51.70, 38.73, 36.35, 32.67, 29.78, 22.94, 21.16, 16.75.

A suspension of -((4-((E)-(2-methoxy-5-methyl-4-((E)-(4-methyl-2-nitrophenyl)diazenyl)phenyl)diazenyl)phenyl)(methyl)amino)-N-(3-(((2R,3R,4S,5S,6R)-3,4,5-trihydroxy-6-((prop-2-yn-1-yloxy)methyl)tetrahydro-2H-pyran-2-yl)oxy)propyl)butanamide and (S)—N-(3-azidopropyl)-2-(3-(5,5-difluoro-7,9-dimethyl-dipyrrolo[1,2-c:2′,1′-f][1,3,2]diazaborinin-4-ium-5-uid-3-yl)propanamido)-6-(dimethylamino)hexanamide in H₂O:DCM (1.5 mL:3.0 mL) was sparged with argon. To this mixture was added CuSO₄ (1.6 mg; 0.010 mmol) and sodium ascorbate (3.2 mg; 0.016 mmol), the round bottom flask was again flushed with argon, and the mixture was stirred overnight at room temperature. TLC: R_{f}=0.2 33% MeOH in EtOAc. After reaction completion as judged by TLC, the mixture was concentrated and enriched using silica gel column chromatography staring 9% MeOH in EtOAc to 20% MeOH in EtOAc to 33% MeOH in EtOAc with 2.5% triethylamine. The material was further purified by HPLC, using an Agilent 1100 Series HPLC equipped with a C8 reverse phase column using an elution rate of 2 mL/min and UV detection at 510 nm. Elution was performed using a gradient of 70-100% 8:2 Acetonitrile:50 mM aqueous ammonium acetate in 50 mM ammonium acetate H₂O over 25 minutes. The fractions collected from 11.5-12 min afforded pure (S)-2-(3-(5,5-difluoro-7,9-dimethyl-dipyrrolo[1,2-c:2′,1′-f][1,3,2]diazaborinin-4-ium-5-uid-3-yl)propanamido)-6-(dimethylamino)-N-(3-(4-((((2R,3S,4S,5R,6R)-3,4,5-trihydroxy-6-(3-(4-((4-((E)-(2-methoxy-5-methyl-4-((E)-(4-methyl-2-nitrophenyl)diazenyl)phenyl)diazenyl)phenyl)(methyl)amino)butanamido)propoxy)tetrahydro-2H-pyran-2-yl)methoxy)methyl)-1H-1,2,3-triazol-1-yl)propyl)hexanamide. Yield: 31%; 7.3 mg. HRMS: [M+H]⁺ calc'd 1291.6485, found 1292.6543. ¹H NMR (600 MHz, Methanol-d₄) δ 7.94 (s, 1H), 7.84 (d, J=9.0 Hz, 2H), 7.76 (d, J=1.8 Hz, 1H), 7.70 (d, J=8.2 Hz, 1H), 7.63-7.54 (m, 1H), 7.52 (s, 1H), 7.37 (s, 1H), 7.36 (s, 1H), 6.96 (d, J=4.0 Hz, 1H), 6.89-6.74 (m, 2H), 6.33 (d, J=4.0 Hz, 1H), 6.19 (s, 1H), 4.70-4.53 (m, 2H), 4.37 (t, J=7.0 Hz, 2H), 4.26 (dd, J=8.8, 5.6 Hz, 1H), 4.23 (d, J=7.8 Hz, 1H), 3.98 (s, 3H), 3.88 (dt, J=10.0, 6.0 Hz, 1H), 3.81 (dd, J=11.0, 1.9 Hz, 1H), 3.67 (dd, J=11.0, 5.5 Hz, 1H), 3.59 (dt, J=10.0, 6.0 Hz, 1H), 3.49 (t, J=7.5 Hz, 2H), 3.37 (ddt, J=14.6, 9.1, 3.2 Hz, 3H), 3.31-3.12 (m, 7H), 3.08 (s, 3H), 2.95-2.80 (m, 2H), 2.71 (d, J=7.0 Hz, 8H), 2.66 (s, 3H), 2.51 (s, 3H), 2.49 (s, 3H), 2.25 (d, J=2.1 Hz, 5H), 2.06 (p, J=6.8 Hz, 2H), 1.94-1.90 (m, 2H), 1.86-1.74 (m, 3H), 1.73-1.54 (m, 3H), 1.47-1.27 (m, 2H). ¹³C NMR (151 MHz, MeOD) δ 173.85, 173.38, 173.05, 159.99, 156.95, 154.62, 152.17, 150.73, 147.51, 145.14, 144.50, 144.46, 144.16, 142.80, 142.41, 135.12, 133.44, 133.14, 132.66, 128.16, 125.53, 124.35, 124.01, 123.84, 120.04, 119.01, 118.59, 116.12, 111.19, 103.03, 99.01, 76.59, 75.46, 73.68, 70.11, 69.46, 67.20, 63.93, 57.57, 55.30, 53.32, 51.23, 47.30, 42.36, 37.36, 36.35, 35.84, 33.98, 32.59, 30.86, 29.67, 28.93, 24.17, 24.07, 22.75, 22.56, 19.77, 15.45, 13.54, 9.83.

### Example 2

Example 2 was synthesized according to procedures analogous to example 1. HRMS: [M+H]⁺³ calc'd for C₇₃H₉₁N₁₅O₁₆ 716.8379 found 716.8394. ¹H NMR (600 MHz, DMSO-d₆) δ 8.9 (d, J=7.6 Hz, 1H), 8.6 (d, J=1.6 Hz, 1H), 8.5-8.4 (m, 2H), 8.3-8.2 (m, 2H), 8.1-8.0 (m, 3H), 7.9-7.8 (m, 3H), 7.4 (s, 1H), 7.4 (s, 1H), 7.3 (d, J=8.0 Hz, 1H), 6.9-6.8 (m, 2H), 6.6-6.4 (m, 6H), 4.6-4.5 (m, 2H), 4.4 (dt, J=19.9, 6.7 Hz, 3H), 4.1 (d, J=7.8 Hz, 1H), 4.0 (s, 3H), 3.9 (s, 3H), 3.7 (ddd, J=11.7, 10.0, 5.1 Hz, 2H), 3.3-3.2 (m, 1H), 3.1 (ddt, J=23.2, 12.9, 6.4 Hz, 3H), 3.1 (s, 2H), 3.0 (t, J=9.3 Hz, 1H), 2.9 (d, J=1.1 Hz, 12H), 2.2 (dt, J=14.1, 7.1 Hz, 4H), 2.1 (s, 6H), 2.0 (t, J=7.0 Hz, 2H), 1.9-1.7 (m, 3H), 1.6 (s, 1H), 1.4 (p, J=9.3, 8.0 Hz, 2H). (Multiple peaks are obscured by the solvent residual peak).

### Example 3

To a solution of (3R,4S,5R,6R)-6-((4-azidobutoxy)methyl)tetrahydro-2H-pyran-2,3,4,5-tetrayl tetraacetate⁴⁰ (200 mg, 0.45 mmol) in dichloromethane (4.5 mL) cooled to 0° C. was added zinc bromide (203 mg, 0.90 mmol) and bromotrimethylsilane (119 μL, 0.90 mmol). The reaction mixture was stirred at 0° C. for 2 hours. After reaction completion, as judged by TLC, the mixture was quenched by the addition of a saturated solution of aqueous sodium bicarbonate (5 mL). The organic layer was separated, and the aqueous layer was extracted twice with dichloromethane (20 mL). The resulting organic layers were combined, dried over sodium sulphate, filtered, and concentrated in vacuo. The crude reaction mixture was used promptly for the next reaction.

The crude reaction mixture was dissolved in a mixture of dichloromethane (2.0 mL) and H₂O (2.0 mL). To the reaction solution was added sequentially tert-butyl (4-hydroxyphenethyl)carbamate (320 mg, 1.35 mmol), potassium carbonate (622 mg, 4.5 mmol), and tetrabutylammonium chloride (184 mg, 0.68 mmol). The resulting reaction mixture was stirred vigorously for 16 hours. After reaction completion, as judged by TLC, the organic layer was separated, and the aqueous layer was extracted twice with dichloromethane (20 mL). The resulting organic layers were combined, dried over sodium sulphate, filtered, and concentrated in vacuo. The crude product was purified using silica gel column chromatography using an elution gradient of 20% EtOAc in hexanes to 40% EtOAc in hexanes to yield pure (2R,3R,4S,5R,6S)-2-((4-azidobutoxy)methyl)-6-(4-(2-((tert-butoxycarbonyl)amino)ethyl)phenoxy)tetrahydro-2H-pyran-3,4,5-triyl triacetate. Yield 78.1 mg; 28%. LRMS MS: [M+H]⁺ calc'd 623.2923, found 623.2834. ¹H NMR (400 MHz, Methanol-d₄) δ 7.2 (d, J=8.5 Hz, 2H), 7.0 (d, J=8.6 Hz, 2H), 5.4 (t, J=9.4 Hz, 1H), 5.3 (d, J=8.0 Hz, 1H), 5.2-5.1 (m, 2H), 4.0 (ddd, J=10.0, 5.2, 2.5 Hz, 1H), 3.6 (dd, J=11.2, 2.6 Hz, 1H), 3.5 (ddd, J=9.5, 6.3, 4.8 Hz, 2H), 3.4 (ddd, J=9.4, 6.9, 4.8 Hz, 1H), 3.2 (dd, J=8.1, 6.6 Hz, 2H), 2.7 (t, J=7.3 Hz, 2H), 2.0 (s, 6H), 2.0 (s, 3H), 1.7-1.6 (m, 4H), 1.4 (s, 9H).

To a solution of (2R,3R,4S,5R,6S)-2-((4-azidobutoxy)methyl)-6-(4-(2-((tert-butoxycarbonyl)amino)ethyl)phenoxy)tetrahydro-2H-pyran-3,4,5-triyl triacetate in methanol (1 mL) was added a sodium methoxide (150 μL of a 0.1 M solution in methanol). The reaction was stirred at 22° C. for 3 hours. After reaction completion, as judged by TLC, the mixture was quenched by the addition of a saturated solution of aqueous sodium bicarbonate (0.1 mL). The reaction mixture was diluted with methanol (4 mL) and subsequently filtered through a thin pad of silica gel. The filtrate was concentrated in vacuo to give a thin clear film. The crude product was dissolved in dichloromethane (2 mL) and cooled to 0° C. Trifluoroacetic acid (0.25 mL) was added to the reaction mixture. After stirring the reaction for 4 hours at 0° C. the mixture was concentrated in vacuo to yield 2-(4-(((2S,3R,4S,5S,6R)-6-((4-azidobutoxy)methyl)-3,4,5-trihydroxytetrahydro-2H-pyran-2-yl)oxy)phenyl)ethan-1-aminium trifluoroacetate. Yield: 100%; 34.9 mg. LRMS MS: [M+H]⁺ calc'd 397.2082, found 397.2052. ¹H NMR (400 MHz, Methanol-d₄) δ 7.2 (d, J=8.6 Hz, 2H), 7.1 (d, J=8.6 Hz, 2H), 4.9-4.9 (m, 1H), 3.8 (dd, J=10.7, 1.5 Hz, 1H), 3.7-3.4 (m, 7H), 3.4-3.3 (m, 2H), 3.3-3.2 (m, 5H), 3.2 (t, J=7.7 Hz, 3H), 2.9 (dd, J=8.8, 6.7 Hz, 3H), 1.8-1.5 (m, 4H).

To a solution of BHQ®2-carboxylic acid (23.4 mg; 0.046 mmol) and diisopropylethyl amine (17 μL; 0.098 mmol) in anhydrous DMF (1.0 mL) was added HBTU (16.3 mg; 0.043 mmol). After stirring for 15 minutes, the reaction mixture was added dropwise to a solution of yield 2-(4-(((2S,3R,4S,5S,6R)-6-((4-azidobutoxy)methyl)-3,4,5-trihydroxytetrahydro-2H-pyran-2-yl)oxy)phenyl)ethan-1-aminium (19.0 mg; 0.039 mmol) in DMF (1 mL). The resulting solution was stirred in the dark for 4 hours. TLC: R_{f} 0.4 in 2% MeOH in CHCl₃. After reaction completion as judged by TLC, the mixture was concentrated, and the crude compound was purified using silica gel column chromatography using a gradient of CH₂Cl₂ to 2% MeOH in CH₂Cl₂ to 5% MeOH in CH₂Cl₂ yielding pure N-(4-(((2S,3R,4S,5S,6R)-6-((4-azidobutoxy)methyl)-3,4,5-trihydroxytetrahydro-2H-pyran-2-yl)oxy)phenethyl)-4-((4-((E)-(2,5-dimethoxy-4-((E)-(4-nitrophenyl)diazenyl)phenyl)diazenyl)phenyl)(methyl)amino)butanamide. Yield: 65%, 22.3 mg. LRMS MS: [M+H]⁺ calc'd 885.3890, found 885.3753. ¹H

To a solution of N-(4-(((2S,3R,4S,5S,6R)-6-((4-azidobutoxy)methyl)-3,4,5-trihydroxytetrahydro-2H-pyran-2-yl)oxy)phenethyl)-4-((4-((E)-(2,5-dimethoxy-4-((E)-(4-nitrophenyl)diazenyl)phenyl)diazenyl)phenyl)(methyl)amino)butanamide (10.0 mg; 0.011 mmol) in THF (1.0 mL) and water (1.0 mL) was added (S)—N′,N′-dimethyl-6-oxo-6-(prop-2-yn-1-ylamino)hexane-1,5-diaminium di(trifluoroacetate) (9.2 mg; 0.022 mmol). To the reaction mixture was added sequentially BTTAA (4.7 mg; 0.011 mmol), anhydrous copper (II) sulfate (3.7 mg; 0.022 mmol), and sodium ascorbate (3.4 mg; 0.022 mmol). The reaction flask was then purged with argon and stirred at 22° C. for 16 hours. After reaction completion, as judged by TLC, the reaction mixture was concentrated in vacuo. The crude product was purified using an Agilent 1100 Series HPLC equipped with a C18 reverse phase column using an elution rate of 2 mL/min and absorbance detection at 550 nm. Elution was performed using a gradient of 20-70% 0.1% TFA in acetonitrile in 0.1% TFA in water over 20 minutes yielding (S)-1-(((1-(4-(((2R,3S,4S,5R,6S)-6-(4-(2-(4-((4-((E)-(2,5-dimethoxy-4-((E)-(4-nitrophenyl)diazenyl)phenyl)diazenyl)phenyl)(methyl)amino)butanamido)ethyl)phenoxy)-3,4,5-trihydroxytetrahydro-2H-pyran-2-yl)methoxy)butyl)-1H-1,2,3-triazol-4-yl)methyl)amino)-6-(dimethylamino)-1-oxohexan-2-aminium trifluoroacetate. Yield: 53%, 7.5 mg. LRMS MS: [M+H]⁺ calc'd 885.3890, found 885.3753.

To a solution of yielding (S)-1-(((1-(4-(((2R,3S,4S,5R,6S)-6-(4-(2-(4-((4-((E)-(2,5-dimethoxy-4-((E)-(4-nitrophenyl)diazenyl)phenyl)diazenyl)phenyl)(methyl)amino)butanamido)ethyl)phenoxy)-3,4,5-trihydroxytetrahydro-2H-pyran-2-yl)methoxy)butyl)-1H-1,2,3-triazol-4-yl)methyl)amino)-6-(dimethylamino)-1-oxohexan-2-aminium trifluoroacetate (3.7 mg; 0.003 mmol) in DMF (1 mL) was added TAMRA-NHS (5.0 mg; 0.009 mmol) and DIPEA (1.3 μL, 0.0072 mmol). The reaction mixture was stirred in the dark for 16 hours. After reaction completion, as judged by TLC, the reaction mixture was concentrated in vacuo. The crude product was purified using an Agilent 1100 Series HPLC equipped with a C18 reverse phase column using an elution rate of 2 mL/min and absorbance detection at 550 nm. Elution was performed using a gradient of 50-70% 0.1% TFA in acetonitrile in 0.1% TFA in water over 20 minutes yielding 5-(((S)-1-(((1-(4-(((2R,3S,4S,5R,6S)-6-(4-(2-(4-((4-((E)-(2,5-dimethoxy-4-((E)-(4-nitrophenyl)diazenyl)phenyl)diazenyl)phenyl)(methyl)amino)butanamido)ethyl)phenoxy)-3,4,5-trihydroxytetrahydro-2H-pyran-2-yl)methoxy)butyl)-1H-1,2,3-triazol-4-yl)methyl)amino)-6-(dimethylamino)-1-oxohexan-2-yl)carbamoyl)-2-(6-(dimethylamino)-3-(dimethyliminio)-3H-xanthen-9-yl)benzoate. Yield: 41%, 1.8 mg. HRMS: [M+H]⁺² calc'd 754.8535, found 754.8536 ¹H NMR (600 MHz, DMSO-d₆) δ 9.3 (s, 1H), 9.0 (d, J=7.9 Hz, 1H), 8.7 (s, 1H), 8.6 (t, J=5.8 Hz, 1H), 8.5-8.4 (m, 2H), 8.4 (d, J=8.0 Hz, 1H), 8.1-8.0 (m, 2H), 7.9 (t, J=5.7 Hz, 1H), 7.9 (s, 1H), 7.8 (d, J=8.7 Hz, 2H), 7.6 (d, J=7.4 Hz, 1H), 7.4 (s, 1H), 7.3 (s, 1H), 7.2 (s, 1H), 7.1 (s, 1H), 7.1 (d, J=8.4 Hz, 2H), 7.0 (s, 1H), 7.0-6.9 (m, 6H), 6.8 (d, J=9.0 Hz, 2H), 4.8 (d, J=7.6 Hz, 1H), 4.5 (q, J=7.8, 7.3 Hz, 1H), 4.3 (dt, J=18.0, 6.4 Hz, 4H), 4.0 (s, 3H), 3.9 (s, 3H), 3.6 (d, J=10.3 Hz, 1H), 3.2 (dt, J=16.6, 8.9 Hz, 14H), 3.1 (t, J=9.1 Hz, 1H), 3.0 (s, 5H), 2.8 (d, J=4.8 Hz, 6H), 2.7-2.6 (m, 2H), 2.1 (t, J=7.2 Hz, 2H), 1.9-1.7 (m, 4H), 1.6 (hept, J=6.6, 5.7 Hz, 2H), 1.5-1.3 (m, 3H). (multiple peaks obscured by DMSO residual signal).

### Example 4

To a solution of N_{α}-Fmoc-N_{ε}-Boc-L-lysine (1.81 g; 3.86 mmol) in DMF (10 mL), DIPEA (679 μL; 3.86 mmol), HBTU (3.66 g; 9.65 mmol) were added separately under Argon at room temperature. To this reaction mixture was added a solution of 3-azido-1-propanamine (464 mg; 4.63 mmol) in DMF (6 mL). The reaction was stirred overnight. After reaction completion, as judged by TLC, the solvent was co-evaporated with toluene. The resulting crude solid was redissolved EtOAc and washed with 10% NaOH, saturated aqueous NaHCO₃ solution and brine. The organic layer was dried over Na₂SO₄, filtered, and concentrated in vacuo. The crude product was purified by silica gel chromatography (toluene/acetone, 3:1) affording (9H-fluoren-9-yl)methyl tert-butyl (6-((3-azidopropyl)amino)-6-oxohexane-1,5-diyl)(S)-dicarbamate as a pale-yellow oil. Yield: 65%, 1.38 g. HRMS: [M+Na]⁺ calc'd for C₂₉H₃₈N₆O₅ 573.2709, found 573.2700.¹H NMR (400 MHz, CDCl₃, 300K) d=7.80-7.71 (m, 2H), 7.58 (d, J=7.3 Hz, 2H), 7.39 (q, J=7.4 Hz, 2H), 7.30 (qd, J=7.4, 1.1 Hz, 2H), 6.43 (s, 1H), 5.62 (s, 1H), 5.56 (d, J=7.7 Hz, 1H), 4.40 (s, 2H), 4.20 (t, J=6.9 Hz, 1H), 4.09 (s, 1H), 3.33 (s, 4H), 3.11 (s, 2H), 1.71 (m, 6H), 1.44 (s, 11H).¹³C NMR (100 MHz, CDCl₃, 300K) d=143.93, 141.50, 127.92, 127.85, 127.24, 125.27, 125.18, 120.16, 120.11, 67.27 49.41, 47.36, 38.80, 37.35, 31.89, 29.66, 28.88, 28.59.

To a solution of (9H-fluoren-9-yl)methyl tert-butyl (6-((3-azidopropyl)amino)-6-oxohexane-1,5-diyl)(S)-dicarbamate (100 mg; 0.182 mmol) in DMF (9 mL), piperidine (900 μL; 10%) was added under Argon at room temperature. The reaction mixture was stirred for 20 mins, then diluted with EtOAc, washed with water, saturated aqueous NaHCO₃ solution and brine. Organic layer was dried over Na₂SO₄ and concentrated. The crude product was purified by silica gel chromatography (EtOAc/MeOH, 15:1-9:1) affording tert-butyl (S)-(5-amino-6-((3-azidopropyl)amino)-6-oxohexyl)carbamate as a pale-yellow oil. Yield: 84%, 50.0 mg. HRMS: [M+H]⁺ calc'd for C₁₄H₂₈N₆O₃ 328.2223, found 329.2218.¹H NMR (400 MHz, MeOD, 300K) d=4.02 (dd, J=7.9, 5.0 Hz, 2H), 3.41 (q, J=6.8 Hz, 2H), 3.32 (dt, J=7.8, 6.5 Hz, 3H), 3.07 (q, J=6.7 Hz, 2H), 1.82 (pd, J=6.7, 4.5 Hz, 2H), 1.71 (ddd, J=13.0, 9.6, 6.6 Hz, 1H), 1.66-1.56 (m, 1H), 1.56-1.50 (m, 2H), 1.46 (s, 9H), 1.45-1.28 (m, 2H).¹³C NMR (101 MHz, MeOD, 300K, 2 rotamers) d=177.56, 177.20, 158.52, 79.82, 58.99, 56.10, 50.25, 50.12, 41.11, 38.83, 37.66, 36.05, 32.53, 30.77, 29.75, 29.66, 28.80, 24.36, 23.96.

To a solution of tert-butyl (S)-(5-amino-6-((3-azidopropyl)amino)-6-oxohexyl)carbamate (12.4 mg, 0.038 mmol) in DMF (1 mL), 5-TAMRA-NHS ester (10 mg, 0.019 mmol) was added, then Et₃N (4 μL, 0.028 mmol) was added to the reaction mixture under Argon at room temperature. The reaction mixture was stirred overnight in the dark. The reaction mixture was diluted with EtOAc, washed with water, saturated aqueous NaHCO₃ solution and brine. The organic layer was dried over Na₂SO₄, filtered, and concentrated. The crude product was purified by silica gel chromatography (DCM/MeOH/Et₃N, 95:5:0.1-85:15:0.1) affording (S)-5-((1-((3-azidopropyl)amino)-6-((tert-butoxycarbonyl)amino)-1-oxohexan-2-yl)carbamoyl)-2-(6-(dimethylamino)-3-(dimethyliminio)-3H-xanthen-9-yl)benzoate as a bright pink oil. Yield: 71%, 9.9 mg. HRMS: [M+H]⁺ calc'd for C₃₉H₄₈N₈O₇ 741.3616, found 741.3620.¹H NMR (400 MHz, MeOD, 300K) d=8.63 (d, J=1.8 Hz, 1H), 8.12 (dd, J=7.9, 1.8 Hz, 1H), 7.39 (d, J=7.9 Hz, 1H), 7.27 (d, J=9.5 Hz, 2H), 7.04 (ddd, J=9.5, 2.5, 1.4 Hz, 2H), 6.94 (d, J=2.4 Hz, 2H), 4.57 (dd, J=8.9, 5.7 Hz, 1H), 3.42 (t, J=6.7 Hz, 2H), 3.31 (s, 12H), 3.19 (q, J=7.4 Hz, 2H), 3.11 (td, J=6.6, 1.9 Hz, 2H), 2.06-1.87 (m, 2H), 1.83 (p, J=6.7 Hz, 2H), 1.57 (td, J=13.2, 7.7 Hz, 3H), 1.46 (s, 9H), 1.31 (t, J=7.3 Hz, 2H). ¹³C NMR (101 MHz, MeOD, 300K, 2 rotamers) d=174.55, 169.20, 158.99, 158.69, 137.35, 136.73, 132.57, 130.69, 129.83, 129.65, 114.95, 114.92, 114.75, 97.37, 55.82, 50.15, 47.86, 40.80, 37.88, 32.79, 29.71, 28.83, 24.51.

To a solution of (S)-5-((1-((3-azidopropyl)amino)-6-((tert-butoxycarbonyl)amino)-1-oxohexan-2-yl)carbamoyl)-2-(6-(dimethylamino)-3-(dimethyliminio)-3H-xanthen-9-yl)benzoate (5.6 mg; 0.008 mmol) in THF (0.5 mL), was added solution of 4-((4-((E)-(2,5-dimethoxy-4-((E)-(4-nitrophenyl)diazenyl)phenyl)diazenyl)phenyl)(methyl)amino)-N-(3-(((2R,3R,4S,5S,6R)-3,4,5-trihydroxy-6-((prop-2-yn-1-yloxy)methyl)tetrahydro-2H-pyran-2-yl)oxy)propyl)butanamide (6.4 mg; 0.008 mmol) in THF (5 mL) was added. The resulting mixture stirred 5 mins at room temperature, after which a solution of CuSO₄·5H₂O (0.6 mg; 0.002 mmol) in H₂O (0.5 mL) and a solution of Sodium ascorbate (0.9 mg; 0.005 mmol) in H₂O (0.5 mL) was added under Argon. The reaction mixture was stirred overnight in dark. The reaction mixture was co-evaporated with toluene and the resulting crude product was purified by silica gel chromatography (DCM/MeOH/Et₃N, 95:5:0.1-90:10:0.1) affording 5-(((S)-6-((tert-butoxycarbonyl)amino)-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate as a purple oil. Yield: 54%, 6.1 mg. HRMS: [M+H]⁺² calc'd for C₇₆H₉₃N₁₅O₁₈ 753.3509, found 753.3509.¹H NMR (600 MHz, MeOD, 300K) d 8.76 (d, J=1.9 Hz, 1H), 8.40-8.27 (m, 4H), 8.05-7.97 (m, 1H), 7.73 (d, J=8.9 Hz, 1H), 7.64 (d, J=7.9 Hz, OH), 7.33 (dd, J=19.4, 9.9 Hz, 2H), 6.94 (dd, J=9.4, 2.6 Hz, 2H), 6.86-6.74 (m, 2H), 6.73 (t, J=3.9 Hz, 2H), 5.31 (s, OH), 4.64-4.52 (m, 2H), 4.47 (t, J=7.1 Hz, 2H), 4.18 (d, J=7.8 Hz, 1H), 3.97 (d, J=3.1 Hz, 4H), 3.90 (d, J=2.6 Hz, 3H), 3.79 (d, J=9.6 Hz, 1H), 3.62 (dd, J=11.5, 5.3 Hz, 2H), 3.56 (td, J=10.6, 9.7, 4.4 Hz, 1H), 3.45 (t, J=7.8 Hz, 2H), 3.39 (p, J=1.6 Hz, 1H), 3.37-3.32 (m, 1H), 3.20-3.09 (m, 17H), 3.06 (d, J=4.9 Hz, 3H), 2.94 (t, J=7.8 Hz, 3H), 2.26-2.17 (m, 2H), 2.16-2.08 (m, 2H), 2.04-1.95 (m, 3H), 1.90 (s, 6H), 1.79-1.67 (m, 5H), 1.64-1.44 (m, 4H), 1.36-1.15 (m, 31H), 0.96 (dd, J=6.8, 4.9 Hz, 1H), 0.85 (dt, J=20.1, 7.0 Hz, 8H).¹³C NMR (100 MHz, CDCl₃, 300K) d 210.41, 162.02, 158.64, 143.02, 131.67, 125.85, 124.68, 114.59, 112.72, 104.41, 97.39, 79.09, 77.99, 77.06, 75.03, 71.54, 57.23, 49.57, 49.43, 49.28, 49.14, 49.04, 49.00, 48.95, 48.86, 48.80, 48.72, 48.57, 40.93, 40.53, 37.79, 30.42, 28.24.

To a solution of 5-(((S)-6-((tert-butoxycarbonyl)amino)-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate (6.1 mg; 0.0041 mmol) in DCM (2 mL) was added TFA (200 μL, 10%). The resulting reaction mixture was stirred 20 mins at room temperature in dark. The reaction mixture concentrated by was co-evaporation with toluene. The resulting crude product was purified by HPLC (30-80% MeCN in H₂O, 0.1% AcOH, over 20 mins, then 95% for 8 mins, XDB-C18) affording 5-(((S)-6-amino-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate as a purple oil. Yield: 68%, 3.8 mg. HRMS: [M+H]⁺³ calc'd for C₇₁H₈₅N₁₅O₁₆ 468.8839, found 468.8843.¹H NMR (600 MHz, MeOD, 300K) d 8.74 (s, 1H), 8.36-8.32 (m, 2H), 8.32-8.29 (m, 1H), 8.01 (dd, J=9.9, 7.5 Hz, 3H), 7.72 (d, J=8.9 Hz, 2H), 7.66 (d, J=7.8 Hz, 1H), 7.33 (d, J=19.6 Hz, 2H), 6.94 (dd, J=9.5, 3.5 Hz, 2H), 6.80 (dt, J=9.4, 2.6 Hz, 2H), 6.76 (d, J=8.9 Hz, RECTIFIED SHEET (RULE 91) 2H), 6.72 (dd, J=4.5, 2.4 Hz, 2H), 4.62 (s, 2H), 4.53 (dd, J=8.7, 6.0 Hz, 1H), 4.46 (t, J=6.9 Hz, 2H), 4.18 (d, J=7.8 Hz, 1H), 3.97 (s, 3H), 3.90 (s, 3H), 3.82 (dt, J=11.1, 6.0 Hz, 1H), 3.78 (d, J=10.7 Hz, 1H), 3.64 (dd, J=11.3, 5.1 Hz, 1H), 3.54 (dt, J=10.8, 6.0 Hz, 1H), 3.47-3.38 (m, 2H), 3.37-3.19 (m, 8H) 3.15 (s, 7H), 3.05 (d, J=3.5 Hz, 3H), 2.95 (t, J=7.7 Hz, 2H), 2.21 (t, J=7.1 Hz, 2H), 2.17-2.07 (m, 2H), 1.99-1.93 (m, 1H), 1.88 (t, J=7.5 Hz, 3H), 1.81-1.66 (m, 4H). ¹³C NMR (150 MHz, MeOD, 300K) d 158.69, 158.63, 154.88, 153.82, 132.45, 132.17, 131.71, 130.73, 127.29, 125.85, 124.69, 115.33, 114.62, 112.64, 104.46, 101.78, 100.88, 97.40, 77.98, 76.86, 75.07, 71.47, 70.82, 68.65, 65.40, 57.21, 57.09, 55.85, 52.68, 49.57, 40.92, 40.54, 38.78, 33.84, 32.25, 31.01, 30.78, 30.35, 28.28, 24.22.

To a solution of 5-(((S)-6-amino-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate (3.8 mg; 0.0027 mmol) in DMF (0.5 mL), was added solution of BCN-NHS ester (0.95 mg; 0.0033 mmol) in DMF (0.5 mL) and DIPEA (1 μL, 0.0054 mmol). The reaction mixture was stirred overnight at room temperature in the dark. The reaction mixture was co-evaporated with toluene and the resulting crude product was purified by HPLC (40-50% MeCN in H₂O, 0.1% AcOH over 20 mins, then 95% for 5 mins, XDB-C18) afforded 5-(((S)-6-(((((1R,8S,9s)-bicyclo[6.1.0]non-4-yn-9-yl)methoxy)carbonyl)amino)-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate as a purple oil. Yield: 56%, 2.4 mg. HRMS: [M+H]⁺² calc'd for C₈₂H₉₇N₁₅O₁₈ 790.8597, found 790.8599. ¹H NMR (600 MHz, DMSO-d₆) δ 8.88 (d, J=7.7 Hz, 1H), 8.57 (s, 1H), 8.44 (d, J=8.3 Hz, 2H), 8.38 (d, J=10.7 Hz, 1H), 8.27 (d, J=8.1 Hz, 1H), 8.22 (d, J=6.1 Hz, 1H), 8.11 (s, 1H), 8.06 (d, J=8.3 Hz, 2H), 7.97 (d, J=8.9 Hz, 1H), 7.88 (t, J=5.8 Hz, 1H), 7.80 (d, J=8.7 Hz, 2H), 7.76-7.66 (m, 1H), 7.58 (d, J=7.7 Hz, OH), 7.44 (s, 1H), 7.37 (s, 1H), 7.30 (d, J=7.8 Hz, 1H), 7.12 (t, J=5.8 Hz, 1H), 6.92-6.81 (m, 2H), 6.49 (qd, J=9.1, 6.6 Hz, 6H), 5.64 (d, J=8.8 Hz, 1H), 5.26 (d, J=3.4 Hz, 1H), 5.09 (s, 2H), 5.06 (dd, J=11.3, 3.4 Hz, 1H), 4.59-4.45 (m, 2H), 4.37 (dt, J=18.8, 7.4 Hz, 3H), 4.22 (t, J=6.2 Hz, 1H), 4.13 (d, J=7.9 Hz, 1H), 4.11-4.06 (m, OH), 4.04 (dd, J=11.4, 5.6 Hz, 1H), 3.93 (s, 3H), 3.72 (t, J=9.9 Hz, 2H), 3.21-3.06 (m, 3H), 3.04 (d, J=5.8 Hz, 4H), 2.96 (t, J=7.5 Hz, 1H), 2.93 (s, 14H), 2.13-2.05 (m, 9H), 2.03 (s, 2H), 1.98 (s, 2H), 1.97-1.93 (m, 2H), 1.89 (d, J=7.9 Hz, 3H), 1.77 (d, J=7.9 Hz, 7H), 1.65 (p, J=7.2, 6.6 Hz, 2H), 1.54-1.36 (m, 3H), 1.34 (s, 3H), 1.22 (s, 10H), 0.84 (ddt, J=27.4, 19.8, 8.7 Hz, 6H). ¹³C NMR (150 MHz, DMSO-d₆) δ 170.02, 153.14, 152.19, 152.03, 144.07, 125.22, 123.58, 111.57, 109.05, 105.55, 99.06, 98.02, 92.50, 76.68, 70.84, 70.08, 66.46, 61.45, 61.26, 56.46, 56.39, 48.19, 40.06, 39.94, 39.86, 39.80, 39.66, 39.52, 39.38, 39.24, 39.10, 38.38, 34.54, 30.47, 29.08, 28.63, 22.78, 22.18, 20.90, 20.63, 20.61, 20.51, 19.57, 17.73

To a solution of 5-(((S)-6-(((((1R,8S,9s)-bicyclo[6.1.0]non-4-yn-9-yl)methoxy)carbonyl)amino)-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate (2.4 mg; 0.0015 mmol) in MeCN (0.5 mL), was added a solution of (S)-2-acetamido-N—((S)-6-amino-1-(((S)-6-amino-1-(((S)-1,6-diamino-1-oxohexan-2-yl)amino)-1-oxohexan-2-yl)amino)-1-oxohexan-2-yl)-6-azidohexanamide (1.4 mg; 0.0023 mmol) in H₂O (0.2 mL). The reaction mixture was stirred in the dark at 37° C. for 16 hours. The reaction mixture was co-evaporated with toluene and the resulting crude product was purified by HPLC (30-70% MeCN in H₂O, 0.1% TFA over 20 mins, then 95% for 5 mins, XDB-C18) affording 5-(((2S)-6-(((((5aS,6R,6aR)-1-(5-acetamido-6-((6-amino-1-((6-amino-1-((1,6-di amino-1-oxohexan-2-yl)amino)-1-oxohexan-2-yl)amino)-1-oxohexan-2-yl)amino)-6-oxohexyl)-1,4,5,5a,6,6a,7,8-octahydrocyclopropa[5,6]cycloocta[1,2-d][1,2,3]triazol-6-yl)methoxy)carbonyl)amino)-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate as a purple oil. Yield: 28%, 810 μg. HRMS: [M+H]⁺⁵ calc'd for C₁₀₈H₁₄₈N₂₆O₂₃ 436.2324, found 436.2326 ¹H NMR (600 MHz, DMSO-d₆) δ 8.80-8.74 (m, 1H), 8.40-8.34 (m, 3H), 8.28 (d, J=47.6 Hz, 1H), 8.12-8.04 (m, 1H), 8.05-7.97 (m, 2H), 7.73 (dd, J=9.3, 3.0 Hz, 2H), 7.66 (d, J=7.9 Hz, 1H), 7.34 (d, J=20.4 Hz, 2H), 6.99-6.90 (m, 2H), 6.83-6.79 (m, 2H), 6.77 (d, J=9.1 Hz, 2H), 6.73 (t, J=2.2 Hz, 2H), 5.31 (d, J=4.9 Hz, 1H), 4.62 (s, 2H), 4.54-4.39 (m, 3H), 4.36-4.22 (m, 5H), 4.18 (d, J=7.8 Hz, 1H), 4.16-4.10 (m, 2H), 4.10-4.02 (m, 1H), 3.98 (s, 3H), 3.90 (d, J=1.9 Hz, 3H), 3.82 (dt, J=11.4, 6.0 Hz, 1H), 3.78 (d, J=10.7 Hz, 1H), 3.68-3.59 (m, 2H), 3.54 (td, J=11.1, 10.3, 4.7 Hz, 1H), 3.45 (t, J=7.5 Hz, 2H), 3.39 (dt, J=3.3, 1.6 Hz, OH), 3.21 (dt, J=13.9, 6.6 Hz, 1H), 3.15 (d, J=1.4 Hz, 8H), 3.14-3.09 (m, 3H), 3.06 (d, J=3.3 Hz, 3H), 2.98 (d, J=18.2 Hz, 1H), 2.91 (q, J=7.1 Hz, 7H), 2.71 (t, J=13.5 Hz, 1H), 2.66 (s, 2H), 2.23 (dt, J=17.0, 7.2 Hz, 2H), 2.18-2.10 (m, 2H), 2.00 (q, J=6.5 Hz, 1H), 1.97-1.93 (m, 4H), 1.88 (dd, J=15.8, 8.5 Hz, 1H), 1.85-1.75 (m, 2H), 1.73 (t, J=6.3 Hz, 1H), 1.65 (h, J=8.5, 7.9 Hz, 3H), 1.57 (s, 1H), 1.51-1.39 (m, 1H), 1.31-1.25 (m, 6H), 0.99 (s, 2H), 0.87 (t, J=6.9 Hz, 8H), 0.87-0.81 (m, 1H).

### 3-Bromopropyl 2,3,4,6-tetra-O-acetyl β-D-glucopyranoside (1)

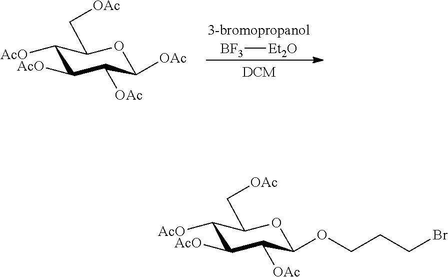

1,2,3,4,6-penta-O-acetyl β-D glucopyranose (12.0 g, 30.3 mmol) was dissolved in anhydrous dichloromethane (125 mL) at RT under and atmosphere of argon. After adding 4 Å molecular sieves (1.5 g) and 3-bromopropanol the reaction mixture was cooled to 0° C. in an ice bath. Boron trifluoride diethyl etherate (15.0 mL, 106 mmol) was added in a dropwise fashion over 5 minutes. The reaction was slowly warmed to 22° C. and stirred for 16 hours. Following reaction completion, as judged by TLC, the reaction mixture was poured onto ice and diluted with dichloromethane (125 mL). The organic layer was separated, and the aqueous layer was extracted with dichloromethane (3×100 mL). The organic layers were combined and subsequently washed with water (150 mL), saturated NaHCO₃ (150 mL), and brine (150 mL). The organic layer was separated, dried over sodium sulfate, filtered, and the filtrate was concentrated in vacuo. The resulting crude mixture was purified by silica gel column chromatography (EtOAc:Hex, 25:75→35:65) to yield compound 1 (5.45 g, 38% yield) as a viscous oil.

¹H NMR (400 MHz, Chloroform-d) δ 5.21 (t, J=9.5 Hz, 1H), 5.08 (t, J=9.7 Hz, 1H), 4.98 (dd, J=9.7, 8.0 Hz, 1H), 4.52 (d, J=8.0 Hz, 1H), 4.27 (dd, J=12.3, 4.8 Hz, 1H), 4.14 (dd, J=12.3, 2.4 Hz, 1H), 3.99 (dt, J=10.0, 5.1 Hz, 1H), 3.80-3.61 (m, 2H), 3.52-3.41 (m, 2H), 2.23-2.10 (m, 2H), 2.09 (s, 3H), 2.07 (s, 3H), 2.03 (s, 3H), 2.01 (s, 3H).

¹³C NMR (101 MHz, Chloroform-d) δ 170.61, 170.19, 169.36, 169.35, 101.03, 72.72, 71.81, 71.26, 68.40, 67.33, 61.91, 32.25, 30.07, 20.72, 20.68, 20.59, 20.57.

Spectral data were in agreement with previous reports

### 3-Azidopropyl 2,3,4,6-tetra-O-acetyl β-D-glucopyranoside (2)

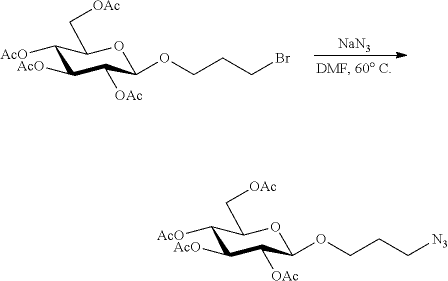

To a solution of Compound 1 (15.5 g, 33.1 mmol) dissolved in DMF (100 mL) was added sodium azide (6.45 g, 99.0 mmol). The reaction mixture was heated to 100° C. in an oil bath and the reaction was stirred vigorously for 16 hours. The reaction mixture was concentrated by azeotroping with toluene. After concentration, the crude oil was dissolved in ethyl acetate (1500 mL) and washed with 0.5 M sodium bicarbonate solution (1500 mL). The organic layer was separated, dried over sodium sulfate, filtered, and the filtrate was concentrated in vacuo to yield compound 2 (14.3 g, quantitative).

¹H NMR (500 MHz, Chloroform-d) δ 5.22 (t, J=9.5 Hz, 1H), 5.14-5.07 (m, 1H), 5.00 (dd, J=9.7, 8.0 Hz, 1H), 4.52 (d, J=8.0 Hz, 1H), 4.28 (dd, J=12.3, 4.8 Hz, 1H), 4.16 (dd, J=12.3, 2.4 Hz, 1H), 4.04-3.89 (m, 1H), 3.72 (ddd, J=10.0, 4.8, 2.4 Hz, 1H), 3.62 (ddd, J=9.8, 7.8, 4.9 Hz, 1H), 3.45-3.30 (m, 2H), 2.10 (s, 3H), 2.07 (s, 3H), 2.04 (s, 3H), 2.02 (s, 3H), 1.96-1.77 (m, 2H).

¹³C NMR (126 MHz, Chloroform-d) δ 170.64, 170.24, 169.39, 169.30, 100.82, 72.78, 71.84, 71.27, 68.39, 66.47, 61.91, 47.92, 28.96, 20.72, 20.65, 20.60, 20.58.

Spectral data were in agreement with previous reports

### 3-Azidopropyl β-D-glucopyranoside (3)

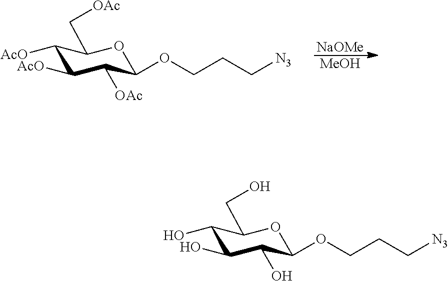

To a solution of compound 2 (6.89 g, 16.0 mmol) in anhydrous methanol was added sodium metal (˜10 mg) under an atmosphere of argon. The reaction was stirred for 4 hours at 22° C. after which it was neutralized using acidic amberlite IR120 resin. The amberlite was removed by filtration and the resulting mixture was concentrated in vacuo to yield pure compound 3 (4.20 g, quantitative)

¹H NMR (600 MHz, Chloroform-d) δ 4.22 (d, J=7.8 Hz, 1H), 3.94 (dt, J=10.4, 6.0 Hz, 1H), 3.90-3.78 (m, 1H), 3.70-3.54 (m, 2H), 3.42 (t, J=6.8 Hz, 2H), 3.33-3.31 (m, 1H), 3.26-3.20 (m, 2H), 3.14 (t, J=7.8 Hz, 1H), 1.84 (q, J=6.5 Hz, 2H).

¹³C NMR (151 MHz, Chloroform-d) δ 104.44, 78.03, 77.94, 75.08, 71.60, 67.54, 62.72, 49.38, 30.24.

Spectral data were in agreement with previous reports [REF]

### 3-Azidopropyl 6-O-Trityl β-D-glucopyranoside (4)

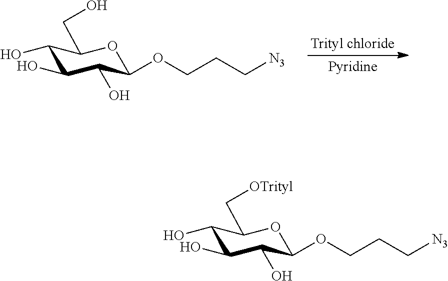

In a 100 mL round bottom flask, compound 3 (1.81 g, 6.87 mmol) was dissolved in pyridine (14.6 mL). Trityl chloride (2.87 g, 10.3 mmol) was added to the reaction mixture portion wise, and the reaction was stirred overnight under an atmosphere of N₂. After completion of the reaction, as judged by TLC, the mixture was co-concentrated with toluene. The resulting crude mixture was purified by silica gel column chromatography (DCM:MeOH, 95:5→9:1) to yield compound 4 (2.48 g, 71% yield) as a white solid.

¹H NMR (400 MHz, Methanol-d₄) δ 7.54-7.46 (m, 6H), 7.34-7.21 (m, 9H), 4.33 (d, J=7.7 Hz, 1H), 4.06 (dt, J=10.1, 6.1 Hz, 1H), 3.78 (dt, J=10.2, 6.2 Hz, 1H), 3.54-3.40 (m, 4H), 3.35 (d, J=1.2 Hz, 1H), 3.30-3.18 (m, 2H), 1.98 (p, J=6.5 Hz, 2H). (1 proton signal obscured by Methanol-d₄ peak)

¹³C NMR (101 MHz, Methanol-d₄) δ 144.14, 128.57, 127.29, 126.61, 103.15, 86.27, 76.92, 75.51, 73.74, 70.64, 66.31, 63.44, 29.02. (1 carbon signal obscured by Methanol-d₄ peak)

HRMS: [M+Na]⁺ calc'd for C₂₈H₃₁N₃NaO₆ 528.2105 found 528.2105

### 3-Azidopropyl 2,3,4-tri-O-(4-methoxybenzyl)-6-O-Trityl β-D-glucopyranoside (5)

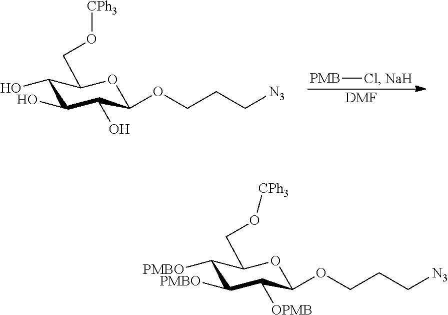

In a 100 mL round bottom flask compound 4 (2.42 g, 4.80 mmol) was dissolved in anhydrous DMF (19.2 mL). After cooling the reaction mixture to 0° C. in an ice bath, sodium hydride (959 mg, 23.98 mmol, 60% dispersion in oil) was added to the solution in a portion wise fashion. After stirring for 5 minutes a thick gel like reaction mixture formed. Para-methoxybenzyl chloride (2.60 mL, 19.2 mmol) was added dropwise using a syringe, after which the reaction was kept at 0° C. for 18 hours. The reaction was quenched by slow addition of a saturated solution of sodium bicarbonate (60 mL). The resulting solution was extracted DCM (250 mL) and the resulting organic layer was separated and washed with water (50 mL) and brine (2×100 mL). The organic layer was then separated, dried over sodium sulfate, filtered, and concentrated under reduced pressure. The crude reaction mixture was subjected to silica gel flash chromatography (Hex:EtOAc, 9:1→8:2) to yield compound 5 (3.24 g, 78% yield) as a white solid.

¹H NMR (600 MHz, Chloroform-d) δ 7.61-7.53 (m, 6H), 7.38-7.28 (m, 13H), 6.95-6.92 (m, 2H), 6.91 (d, J=8.6 Hz, 2H), 6.84-6.79 (m, 2H), 6.75 (d, J=8.6 Hz, 2H), 4.92 (d, J=10.6 Hz, 1H), 4.86 (d, J=10.4 Hz, 1H), 4.77 (dd, J=10.6, 7.4 Hz, 2H), 4.66 (d, J=10.0 Hz, 1H), 4.45 (d, J=7.6 Hz, 1H), 4.33 (d, J=9.9 Hz, 1H), 4.16 (ddd, J=11.7, 6.1, 3.3 Hz, 1H), 3.85 (s, 3H), 3.84 (s, 3H), 3.83-3.80 (m, 4H), 3.76 (ddd, J=10.0, 7.2, 5.4 Hz, 1H), 3.60 (ddd, J=9.1, 5.0, 3.1 Hz, 2H), 3.57-3.51 (m, 3H), 3.43 (ddd, J=9.8, 4.0, 1.9 Hz, 1H), 3.27 (dd, J=10.1, 4.0 Hz, 1H), 2.15-1.99 (m, 2H).

¹³C NMR (151 MHz, Chloroform-d) δ 159.37, 159.33, 159.27, 144.01, 130.88, 130.77, 130.16, 129.96, 129.80, 129.79, 128.96, 127.91, 127.10, 113.96, 113.95, 113.72, 103.72, 86.42, 84.53, 82.34, 77.64, 75.74, 74.80, 74.75 (2 signals), 66.39, 62.45, 55.40 (2 signals), 55.37, 48.59, 29.46.

HRMS: [M+NH₄]⁺ calc'd for C₅₂H₅₉N₄O₉ 883.4277 found 883.4269

### 3-Azidopropyl 2,3,4-tri-O-(4-methoxybenzyl) β-D-glucopyranoside (6)

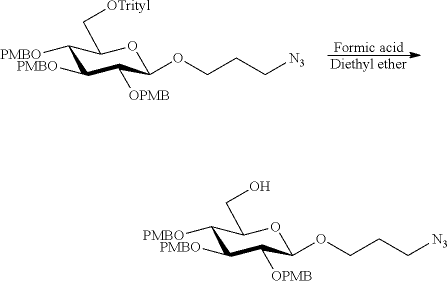

A solution of compound 5 (3.11 g, 3.59 mmol) in diethyl ether (48 mL) was cooled to 0° C. under an atmosphere of nitrogen gas. Formic acid (99%) (40 mL) was added slowly and the reaction mixture was stirred at room temperature for 2 hours. After reaction completion, as judged by TLC, the crude mixture was poured into water (250 mL) and subsequently diluted with dichloromethane (250 mL). The organic layer was separated and subsequently washed with saturated NaHCO₃ (125 mL) and brine (125 mL). The organic layer was separated, dried over sodium sulfate, filtered, and the filtrate was concentrated in vacuo. The crude reaction mixture was subjected to silica gel column chromatography (Hex:EtOAC, 8:2→7:3) for to yield compound 6 (1.41 g, 63% yield) as an amorphous solid.

¹H NMR (600 MHz, Chloroform-d) δ 7.30-7.24 (m, 4H), 7.20 (d, J=8.6 Hz, 2H), 6.90-6.85 (m, 6H), 4.88 (d, J=10.6 Hz, 1H), 4.83 (d, J=10.6 Hz, 1H), 4.80 (d, J=10.6 Hz, 1H), 4.75 (d, J=10.5 Hz, 1H), 4.68 (d, J=10.6 Hz, 1H), 4.50 (d, J=10.6 Hz, 1H), 4.45-4.33 (m, 2H), 4.27 (dd, J=12.1, 3.5 Hz, 1H), 3.97 (dt, J=9.9, 5.9 Hz, 1H), 3.82-3.79 (m, 9H), 3.70-3.61 (m, 2H), 3.54-3.46 (m, 2H), 3.45-3.36 (m, 3H), 2.03-1.78 (m, 2H).

¹³C NMR (151 MHz, Chloroform-d) δ 159.52, 159.37, 159.29, 130.63, 130.44, 129.98, 129.81, 129.77, 129.61, 114.01, 113.92, 113.89, 103.65, 84.36, 81.93, 76.71, 75.47, 74.74, 74.71, 72.71, 66.79, 62.44, 55.37, 55.34×2, 48.33, 29.30.

HRMS: [M+NH₄]⁺ calc'd for C₃₃H₄₅N₄O₉ 641.3181 found 641.3176

### 3-Azidopropyl 2,3,4-tri-O-(4-methoxybenzyl)-6-O-propargyl β-D-glucopyranoside (7)

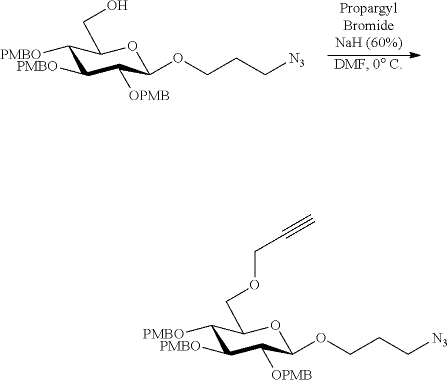

A solution of compound 6 (1.37 g, 2.19 mmol) in anhydrous dimethylformamide (11 mL) was cooled to 0° C. in an ice bath. Sodium hydride (60% dispersion in oil) (175 mg, 4.38 mmol) was added portion wise to the reaction mixture over 15 minutes. Keeping the reaction mixture at 0° C., propargyl bromide (366 μL, 3.28 mmol) was added in a dropwise fashion. The reaction was stirred for 4 hours at 0° C. After reaction completion, as judged by TLC, the reaction was quenched by the addition of methanol (5 mL) and subsequently diluted with dichloromethane (200 mL). The crude mixture was washed with saturated sodium bicarbonate (150 mL), water (25 mL), and brine (2×150 mL). The organic layers were separated, dried over sodium sulfate, filtered, and the filtrate was concentrated in vacuo. The resulting crude product was purified by silica gel column chromatography using a gradient of (Hex:EtOAC, 80:20→70:30) to yield compound 7 (1.35 g, 93% yield) as a clear oil.

¹H NMR (600 MHz, Chloroform-d) δ 7.31-7.23 (m, 6H), 6.91-6.85 (m, 6H), 4.90-4.72 (m, 4H), 4.68 (d, J=10.7 Hz, 1H), 4.63 (d, J=10.4 Hz, 1H), 4.37 (dd, J=7.8, 1.3 Hz, 1H), 4.28 (ddd, J=16.0, 2.4, 1.0 Hz, 1H), 4.20 (ddd, J=15.9, 2.3, 1.0 Hz, 1H), 4.06-3.99 (m, 1H), 3.88-3.73 (m, 11H), 3.70-3.60 (m, 1H), 3.63-3.54 (m, 2H), 3.49-3.39 (m, 4H), 2.48-2.41 (m, 1H), 1.98-1.90 (m, 2H).

¹³C NMR (151 MHz, Chloroform-d) δ 159.31, 159.26, 159.16, 130.82, 130.53, 130.35, 129.73, 129.71, 129.50, 113.83, 113.82, 113.79, 103.64, 84.34, 81.92, 79.67, 75.38, 74.80, 74.68, 74.63, 74.57, 68.34, 66.61, 58.67, 55.31, 55.30, 48.36, 29.27.

HRMS: [M+NH₄]⁺ calc'd for C₃₆H₄₇N₄O₉ 679.3338 found 679.3336

### 3-Azidopropyl 6-O-propargyl β-D-glucopyranoside (8)

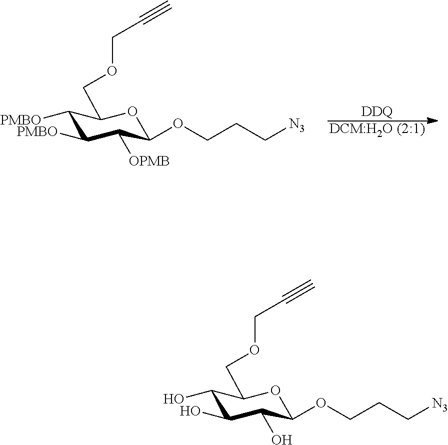

Compound 7 (1.05 g. 1.57 mmol) was dissolved in DCM (10 mL). After addition of H₂O (5 mL), 2,3-Dichloro-5,6-dicyano-1,4-benzoquinone (1.61 g, 7.09 mmol) was added all at once. The reaction was stirred vigorously for 2 hours after which 50 mL of saturated NaHCO₃ was added to quench the reaction. After removing the solvent in vacuo, the crude mixture was suspended in a mixture of dichloromethane and methanol (10:1, 75 mL) and filtered through a pad of celite. The resulting crude mixture was purified by silica gel column chromatography (DCM:MeOH, 95:5→9:1) to yield compound 8 (375 mg, 79% yield) as a clear oil.

¹H NMR (400 MHz, Methanol-d₄) δ 4.36-4.17 (m, 3H), 4.07-3.87 (m, 2H), 3.77-3.61 (m, 2H), 3.56-3.37 (m, 3H), 3.35-3.26 (m, 2H), 3.19 (dd, J=9.0, 7.8 Hz, 1H), 2.86 (t, J=2.4 Hz, 1H), 2.13-1.67 (m, 2H).

¹³C NMR (101 MHz, Methanol-d4) δ 103.07, 79.22, 76.60, 75.37, 74.57, 73.60, 70.23, 68.90, 66.33, 58.07, 48.01, 28.90.

### 3-Amido-BHQ-1 6-O-propargyl β-D-glucopyranoside (9)

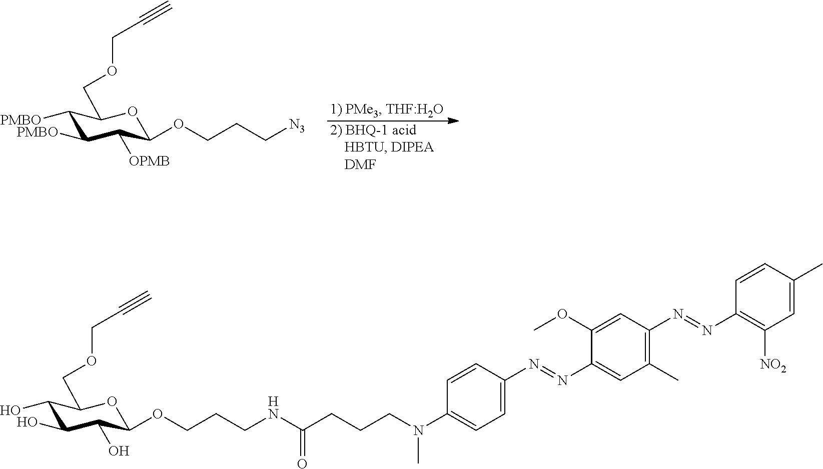

Compound 8 (157.1 mg, 0.521 mmol) was dissolved in a 2:1 mixture of THF and water (3.6 mL). A 1 M solution of trimethyl phosphine in THF (1.56 mL, 1.56 mmol) was added in a dropwise fashion to the reaction mixture. The mixture was stirred under an atmosphere of argon for 3 hours. Upon reaction completion as judged by TLC, the reaction was concentrated in vacuo to give the crude amine product which was used without purification.

To a solution of BHQ®1-carboxylic acid (329.1 mg; 0.625 mmol) and diisopropylethylamine (201.7 mg; 1.56 mmol) in anhydrous DMF (4.3 mL) was added HBTU (237.0 mg; 0.625 mmol). After stirring for 30 minutes, the reaction mixture was added dropwise to a solution of the Staudinger reduction product (143.4 mg, 0.521 mmol) in DMF (2 mL). The resulting solution was stirred in the dark for 3 hours. After reaction completion as judged by TLC, the mixture was concentrated, and the crude compound was purified using silica gel column chromatography (Chloroform:MeOH, 95:5→9:1) yielding pure compound 9 as a green amorphous solid (271.8 mg, 67% yield).

¹H NMR (600 MHz, DMSO-d₆) δ 7.95 (s, 1H), 7.84-7.78 (m, 3H), 7.77 (d, J=8.2 Hz, 1H), 7.71-7.67 (m, 1H), 7.52 (s, 1H), 7.29 (s, 1H), 6.88 (d, J=9.3 Hz, 2H), 5.11-5.03 (m, 2H), 5.00 (d, J=5.0 Hz, 1H), 4.31-4.09 (m, 3H), 3.93 (s, 3H), 3.83-3.69 (m, 2H), 3.47 (dt, J=8.9, 6.6 Hz, 4H), 3.42 (t, J=2.4 Hz, 1H), 3.28 (ddd, J=9.8, 6.5, 1.8 Hz, 1H), 3.21-3.09 (m, 3H), 3.07 (s, 3H), 3.05-2.98 (m, 1H), 2.96 (ddd, J=9.0, 7.8, 4.8 Hz, 1H), 2.64 (s, 3H), 2.50 (s, 3H), 2.16 (t, J=7.3 Hz, 2H), 1.80 (p, J=7.4 Hz, 2H), 1.68 (p, J=6.7 Hz, 2H).

¹³C NMR (151 MHz, DMSO) δ 171.97, 154.80, 152.34, 150.55, 146.85, 145.18, 143.98, 143.04, 142.76, 134.37, 132.73, 125.98, 124.74, 120.55, 118.95, 111.89, 103.19, 99.61, 80.94, 77.52, 77.05, 75.66, 73.81, 70.51, 69.69, 67.18, 58.29, 56.40, 51.70, 38.73, 36.35, 32.67, 29.78, 22.94, 21.16, 16.75.

HRMS: [M+H]⁺ calc'd for C₃₈H₄₈N₇O₁₀ 762.3457, found 762.3467

### 3-Azidopropylamide BODIPY-FL (15)

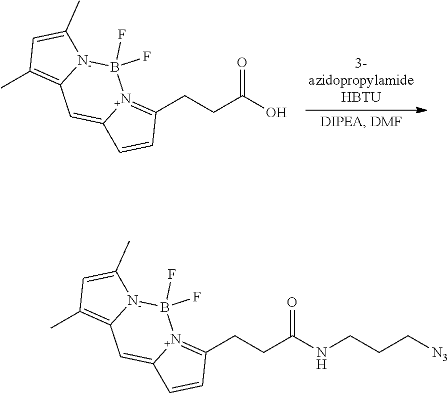

14 (69.5 mg, 0.238 mmol) was dissolved in anhydrous DMF (2.4 mL). HBTU (94.7 mg, 0.262 mmol) and 3-azidopropylamine (94 μL, 0.952 mmol) were added subsequently to the reaction. After the reaction was stirred for 2 hours the solvent was removed under reduced pressure and the resulting mixture was diluted with dichloromethane (75 mL). The resulting solution was washed with a saturated solution of NaHCO₃ (25 mL), H₂O (50 mL), and brine (25 mL). The organic layer was separated, dried over sodium sulfate, filtered and the filtrate was dried in-vacuo. The resulting crude product was purified using silica gel flash column chromatography (EtOAc:DCM, 10:90) yielding pure 15 as an orange film (69.0 mg, 77% yield).

¹H NMR (600 MHz, Chloroform-d) δ 7.13 (s, 1H), 6.92 (d, J=4.0 Hz, 1H), 6.32 (d, J=4.0 Hz, 1H), 6.17 (s, 1H), 5.96 (s, 1H), 3.35-3.27 (m, 4H), 3.23 (t, J=6.8 Hz, 2H), 2.70 (t, J=7.4 Hz, 2H), 2.60 (s, 3H), 2.29 (s, 3H), 1.73-1.67 (m, 2H).

¹³C NMR (151 MHz, Chloroform-d) δ 171.93, 160.65, 156.79, 144.24, 135.26, 133.31, 128.20, 123.84, 120.63, 117.51 (d, J=3.9 Hz), 49.01, 36.86, 35.91, 28.70, 24.91, 14.99, 11.35.

HRMS: [M+Na]⁺ Calc'd for C₁₇H₂₁BF₂N₆NaO₂ 397.1733, found 397.1733

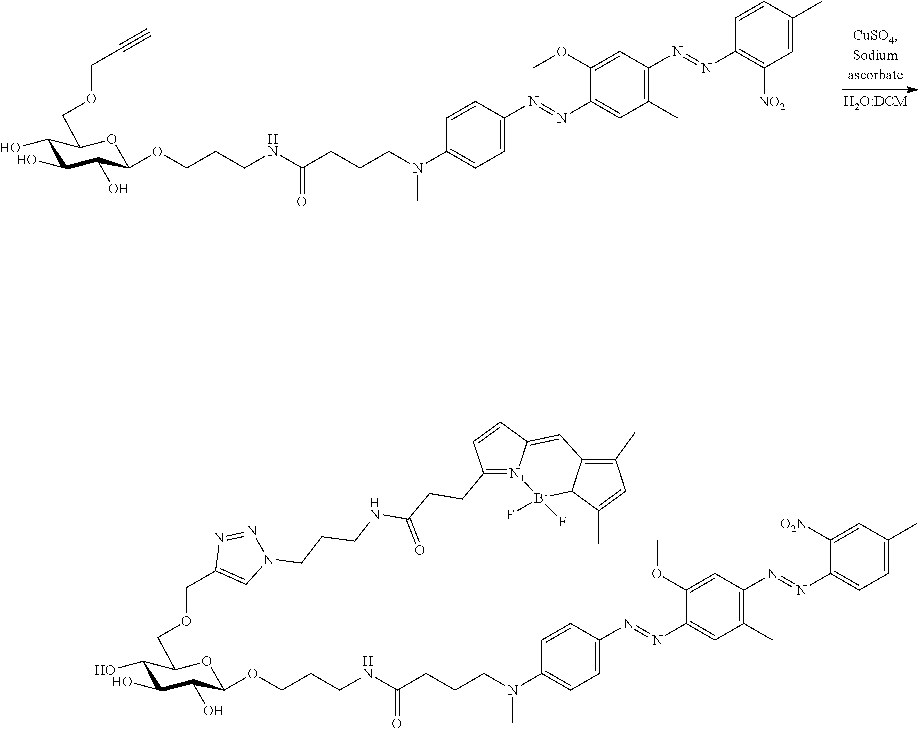

In a 250 mL round bottom flask 9 (138.1 mg, 0.177 mmol) and compound 15 (66.3 mg, 0.177 mmol) were dissolved DCM (20.5 mL) and water (12.5 mL). After purging with nitrogen for 5 minutes with vigorous stirring, copper sulfate (8.5 mg, 0.053 mmol) and sodium ascorbate (21.0 mg, 0.106 mmol) were added in one portion. The resulting reaction was stirred overnight under an atmosphere of nitrogen. After reaction completion as judged by TLC, the organic layer was separated from the aqueous layer and subsequently concentrated in vacuo. The crude mixture was purified by silica gel column chromatography (EtOAC:MeOH, 8:2) to yield pure MDFL1 as a darkly coloured amorphous solid (167 mg g, 82% yield).

¹H NMR (600 MHz, DMSO-d₆) δ 8.07 (s, 1H), 8.02 (t, J=5.6 Hz, 1H), 7.95-7.91 (m, 1H), 7.83-7.73 (m, 4H), 7.73-7.64 (m, 2H), 7.51 (d, J=0.8 Hz, 1H), 7.28 (s, 1H), 7.07 (d, J=4.0 Hz, 1H), 6.90-6.76 (m, 2H), 6.34 (d, J=4.0 Hz, 1H), 6.29 (s, 1H), 5.01 (dd, J=7.6, 5.1 Hz, 2H), 4.96 (d, J=4.9 Hz, 1H), 4.60-4.49 (m, 2H), 4.34 (t, J=7.0 Hz, 2H), 4.13 (d, J=7.8 Hz, 1H), 3.91 (s, 3H), 3.81-3.62 (m, 2H), 3.56-3.41 (m, 4H), 3.27 (ddd, J=9.7, 6.2, 1.8 Hz, 2H), 3.23-2.99 (m, 12H), 2.95 (ddd, J=9.0, 7.8, 4.7 Hz, 1H), 2.62 (d, J=0.8 Hz, 3H), 2.46 (s, 3H), 2.25 (s, 3H), 2.14 (t, J=7.2 Hz, 2H), 1.95 (q, J=6.9 Hz, 2H), 1.82-1.74 (m, 2H), 1.65 (q, J=6.6 Hz, 2H). (3 proton signals are obscured by the DMSO residual peak).

¹³C NMR (151 MHz, DMSO-d₆) δ 171.99, 171.44, 159.62, 158.20, 154.81, 152.33, 150.56, 146.85, 145.21, 144.55, 144.52, 143.98, 143.04, 142.77, 134.91, 134.37, 133.42, 132.72, 129.36, 125.97, 125.79, 124.73, 124.29, 120.72, 120.55, 118.96, 117.03, 111.88, 103.25, 99.65, 77.09, 75.94, 73.83, 70.50, 70.14, 67.20, 64.46, 56.42, 51.69, 47.62, 38.71, 36.37, 36.28, 34.25, 32.68, 30.39, 29.80, 24.44, 22.94, 21.16, 16.74, 14.97, 11.46.

HRMS: [M+Na]⁺ Calc'd for C₅₅H₆₈BF₂N₁₃NaO₂ 1158.5124, found 1158.5140

### Example 1

### Fluorescence Spectrum of Substrate 1

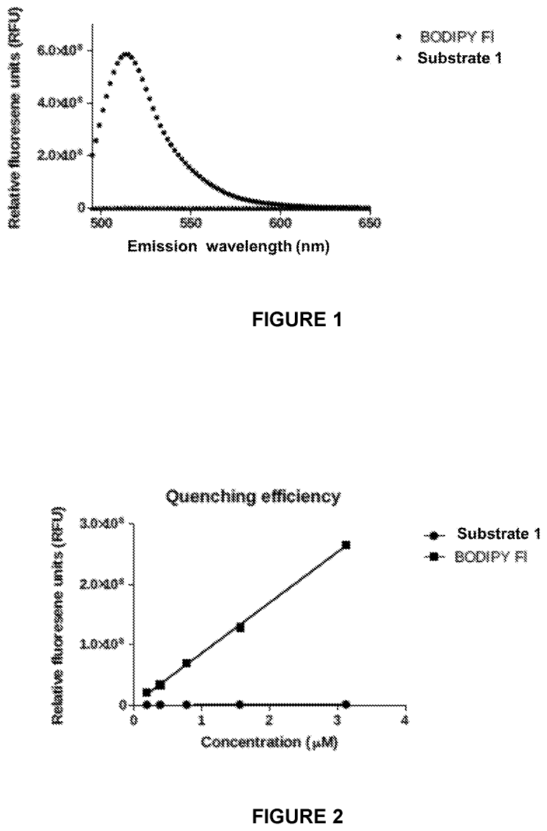

Fluorescence emission spectra for substrate 1 and BODIPY® FL-acid: The emission spectra were obtained for substrate 1 and BODIPY® FL-acid in citrate-phosphate buffer (150 mM; pH 5.4) containing 1% DMSO and 0.25% taurodeoxycholate. Data were collected using a Molecular Devices plate reader (Varian Inc). FIG. 1 shows the plot of fluorescence emission intensity versus wavelength for substrate 1 (6.25 μM) and BODIPY®Fl (6.25 μM) in the assay buffer used (citrate-phosphate buffer 150 mM; pH 5.4). The excitation wavelength used was 485 nm and the emission wavelength was 513 nm. The intact substrate 1 shows strikingly little fluorescence, compared to that of the free fluorophore BODIPY®FL-acid.

### Example 2

### Fluorescence Quenching Efficiency of Substrate 1

Determination of the quenching efficiencies for substrate 1 using concentration dependent variation in the fluorescence emission of substrate 1 and its corresponding fluorophore BODIPY® FL-acid: The quenching efficiencies were determined by comparing the fluorescence emission standard curves of substrate 1 and comparing it to that of BODIPY® Fl-acid. FIG. 2 shows the plot of the fluorescence emission intensity versus concentration of substrate 1 and BODIPY® Fl. The data show that substrate 1 has a measured quenching efficiency of greater than 99.7%.

### Example 3

### Kinetic Assay for Substrate 1

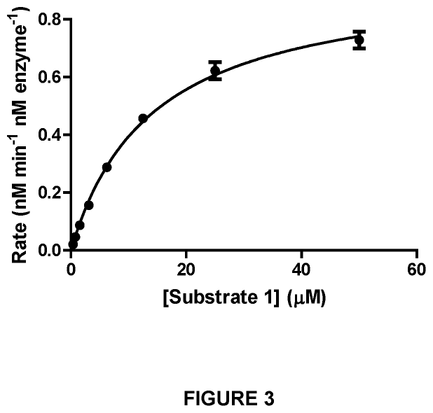

In-vitro kinetic assay for substrate 1. Continuous assays were performed at 30° C. in citrate-phosphate buffer (150 mM; pH 5.4) containing 0.25% taurodeoxycholate. The concentrations of substrate 1 used in assays were 0.39, 0.78, 1.56, 3.13 6.25, 12.5, 25 and 50 μM. Reactions were initiated by the addition of purified recombinant GCase (2.5 nM, R&D Systems) and the reaction was monitored continuously over 1200 seconds to determine the initial rate of change of fluorescence. Standard curves were constructed using BODIPY® Fl-acid using the same buffer conditions. FIG. 3 shows the plot of rate (nM product min⁻¹ nM enzyme⁻¹) versus the concentration of substrate (μM) for substrate 1. Assays were carried out in triplicate. Based on these data, it was determined that the second order rate constant for cleavage of this substrate k_{cat}/Kₘ=78±2 min⁻¹ mM⁻¹.

### Example 4

### Evaluation of Substrate 1 in SK-N-SH Cells

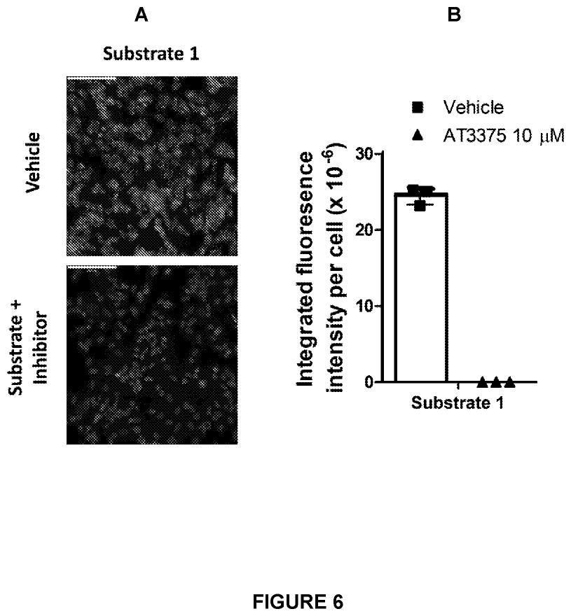

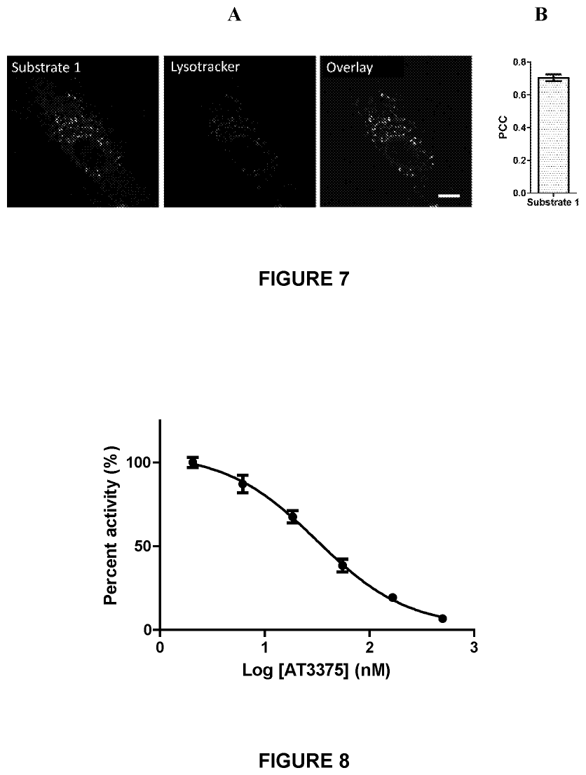

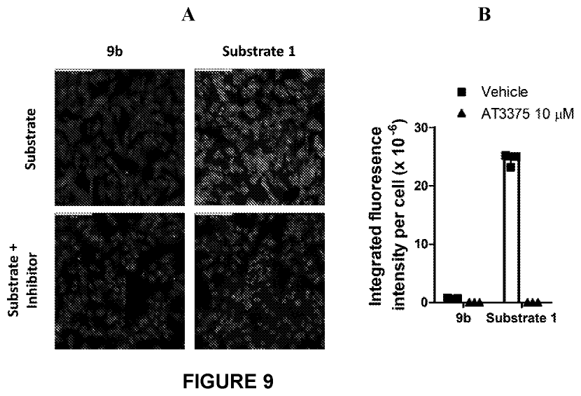

Substrate 1 was evaluated in the human neuronal cell culture model cells SK-N-SH. High content imaging of SK-N-SH cells treated with DAPI, to stain nuclei, and 5 μM of substrate 1 revealed fluorescence in a punctuate pattern consistent with a lysosomal distribution (FIGS. 6A-B and FIGS. 7A-B). Cells plated in a 96-well clear-bottom Corning plate were treated with vehicle, 5 μM of substrate 1 alone or 5 μM of substrate 1 with 10 μM of the selective GCase inhibitor AT3375. After incubation at 37° C. and 5% CO₂ for 2 h, the media was removed and the cells were washed. The plate was then imaged using a Molecular Devices ImageXpress XLS High-Content Imager Importantly, cells treated with the inhibitor showed a significant reduction of the punctuated fluorescent signal (FIGS. 6A-B), demonstrating that substrate 1 turnover is dependent upon the enzymatic activity of GCase. The substrate 1 treated cells show punctuated signal supporting the lysosomal localization of the substrate (FIGS. 7A-B). The merged image of the FITC and TRITC channels show significant co-localization with a Pearson's correlation coefficient value of 0.72, suggesting substrate 1 is turned over by GCase localized within lysosomes (FIGS. 7A-B).

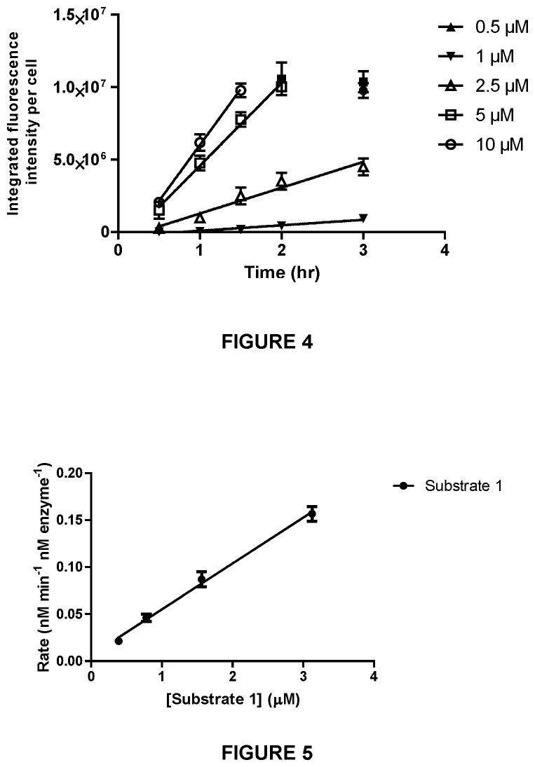

Using substrate 1, time dependent processing was established in SK-N-SH cells. High content imaging of cells treated with DAPI, to stain nuclei, and different concentrations of substrate 1 revealed increasing fluorescence as a function of time. The fluorescence associated with cleavage of substrate 1 was present in a punctate pattern consistent with a lysosomal distribution. By integrating the fluorescence intensity from imaged fields, it was possible to observe a quantitative increase in fluorescence as a function of time (FIG. 4). Next, it was established that there is a dose-dependent cellular fluorescence of substrate 1 where a linear increase in fluorescence was observed that depended on the concentration of substrate 1 (FIG. 5). While these data reveal substrate 1 is processed in cells to result in the fluorescent product, subsequent experiments showed that the observed fluorescence was dependent on GCase activity.

### Example 5

### Measurement of IC₅₀ Values of GCase Inhibitors in Live Cells

Substrate 1 can be used to measure the potency of a GCase inhibitor in live cells. High content imaging of SK-N-SH cells incubated for 2 h with DAPI, to stain nuclei, 5 μM of substrate 1 and varying concentrations of the selective GCase inhibitor AT3375 revealed decreasing fluorescence signal with increasing inhibitor concentration (FIG. 8). Cells were plated in a 96-well clear-bottom Corning plate and treated with vehicle, 5 μM of substrate 1 alone or 5 μM of substrate 1 with different concentrations of the GCase inhibitor AT3375 (triplicates). After incubation at 37° C. and 5% CO₂ for 2 h, the media was removed, and the cells were washed. The plate was then imaged using a Molecular Devices ImageXpress XLS High-Content Imager. From the images, the total mean integrated intensity was measured for the FITC channel and divided by the number of cells within the imaging site as determined by the DAPI staining. Integration of the mean fluorescence intensity per cell from randomly selected fields (9 sites per well), the percentage of activity (no inhibitor as a 100% activity control) was plotted against the log of the AT3375 inhibitor concentration to determine the IC₅₀ in cells. These data reveal substrate 1 can be used to measure the effect of an inhibitor on GCase in live cells.

### Example 6

### Substrate 1 is a Bright and Robust Tool to Measure GCase Activity in Live Cells

Poor cellular retention of signal is one of the problems limiting the application of fluorescence quenched substrates suitable for live cell imaging. To determine the cellular retention of substrate 1, signal stability as a function of time was examined in comparison to substrate 9b:

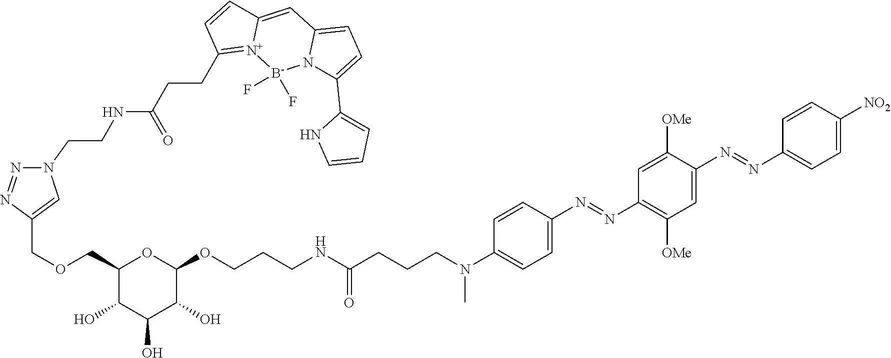 [SMILES: COc1cc(/N=N/c2ccc([N+](=O)[O-])cc2)c(OC)cc1/N=N/c1ccc(N(C)CCCC(=O)NCCCO[C@@H]2O[C@H](COCc3cn(CCNC(=O)CCC4=[N+]5C(=Cc6ccc(-c7ccc[nH]7)n6[B-]5(F)F)C=C4)nn3)[C@@H](O)[C@H](O)[C@H]2O)cc1]

3-(3-((2-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)-butanamido)propoxy)-3,4,5-trihydroxytetrahydro-2H-pyran-2-yl)methoxy)methyl)-1H-1,2,3-triazol-1-yl)ethyl)amino)-3-oxopropyl)-5,5-difluoro-7-(1H-pyrrol-2-yl)-5H-dipyrrolo[1,2-c:2′,1′-f][1,3,2]diazaborinin-4-ium-5-uide.

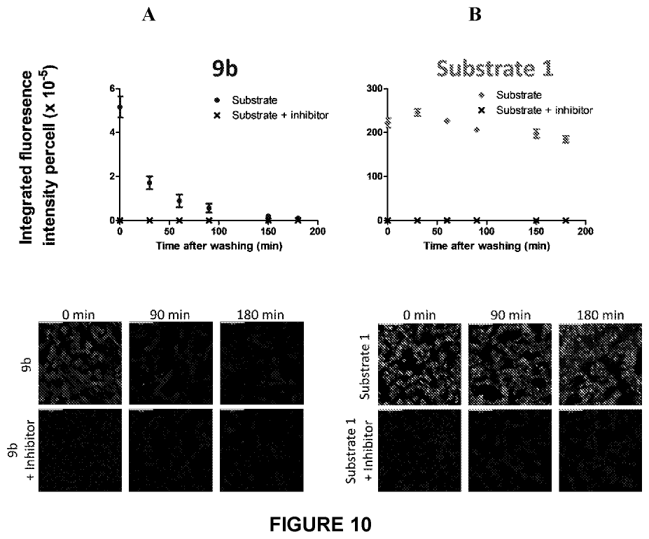

SK-N-SH cells were treated with either substrate at 10 μM for 2 hours in the presence or absence of AT3375 10 μM. After incubation the cells were washed, and media supplemented with AT3375 10 μM was dispensed in all wells. The cells were then read directly using the Wide-Field ImageXpress Micro XLS (Molecular Devices) system. The plate was imaged at time points (0 h, 0.5 h, 1 h, 1.5 h, 2.5 h, 3 h) and signal intensity was measured. The signal intensity from substrate 1 was seen to be stable at all time points measured (FIGS. 10A-B). In comparison, the fluorescent signal from substrate 9b showed decay within the first 30 minutes after replacement of media (FIGS. 10A-B). Demonstrating that the fluorescent product of substrate 1 is robustly retained within cells as shown by the increased fluorescence intensity illustrated (FIGS. 10A-B).

### Example 7

### Substrate 3 is Turned Over Rapidly

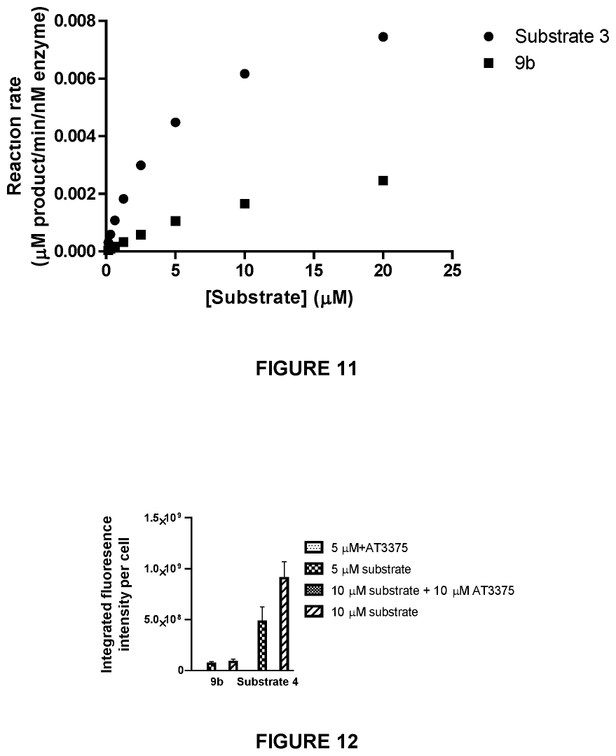

Comparative In-vitro kinetic assay for substrate 3 and 9b. Continuous assays were performed at 30° C. in citrate-phosphate buffer (150 mM; pH 5.4) containing 0.25% taurodeoxycholate. The concentrations of substrates 3 and 9b used in assays were 0.156, 0.313, 0.625, 1.25, 2.5, 5, 10, and 20 μM. Reactions were initiated by the addition of purified recombinant GCase (2.5 nM, R&D Systems) and the reaction was monitored continuously over 1200 seconds to determine the initial rate of change of fluorescence. Standard curves were constructed using TAMRA and BODIPY-576 using the same buffer conditions. FIG. 11 shows the plot of rate (nM product min⁻¹ nM enzyme⁻¹) versus the concentration of substrate (μM) for substrates 3 and 9b. Assays were carried out in triplicate. Based on this data, it was determined that the second order rate constant for cleavage of substrate 3 was more than 5-fold greater than 9b (k_{cat}/Kₘ=1360 min⁻¹ mM⁻¹ vs. k_{cat}/Kₘ=261 min⁻¹ mM⁻¹). Accordingly, fluorescence quenched substrate 3 shows a greatly improved second order rate constant as is evidence by the turnover at low substrate concentrations.

### Example 8

### Evaluation of Substrate 4 in Fixed SK-N-SH Cells

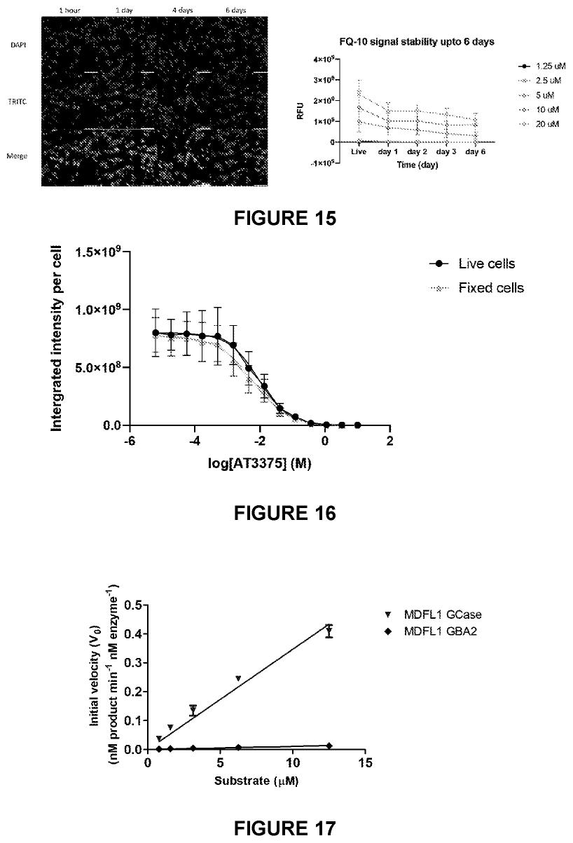

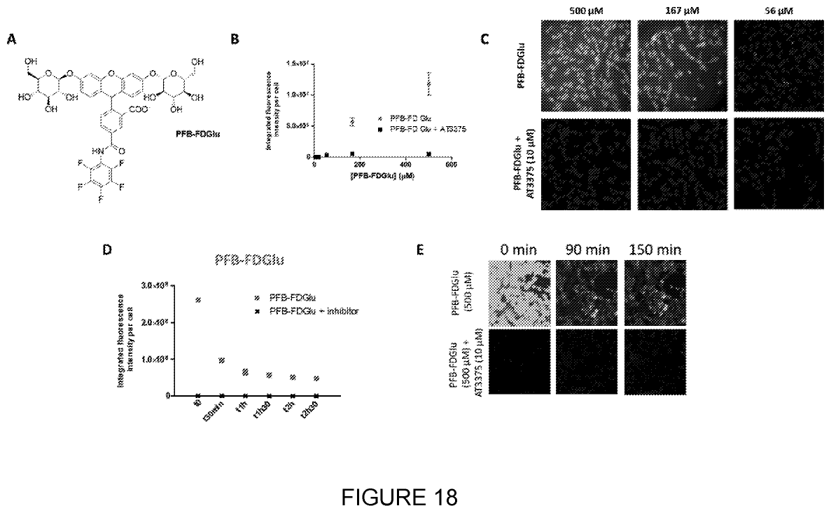

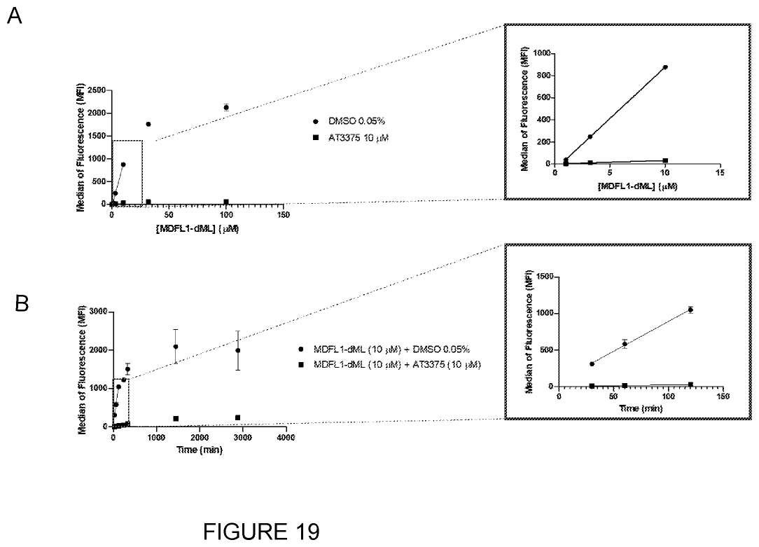

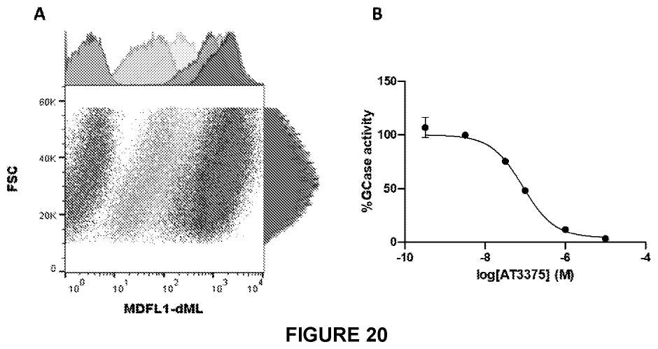

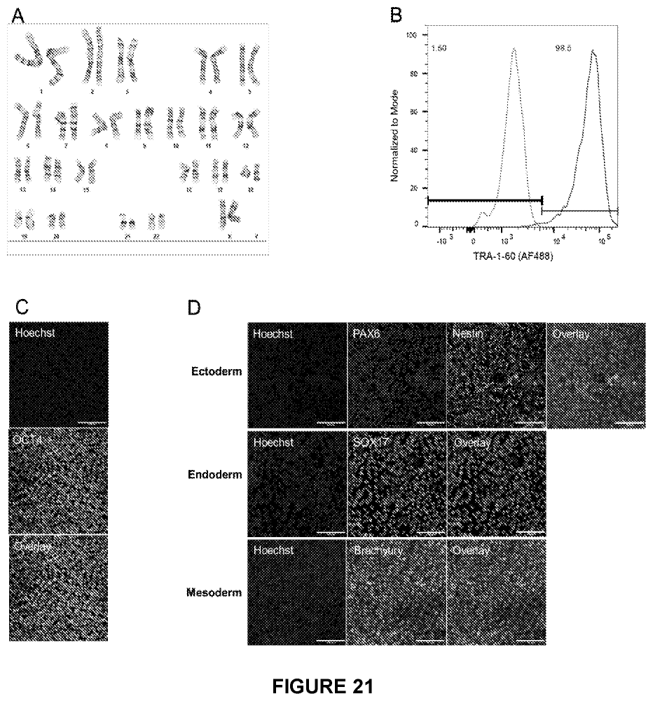

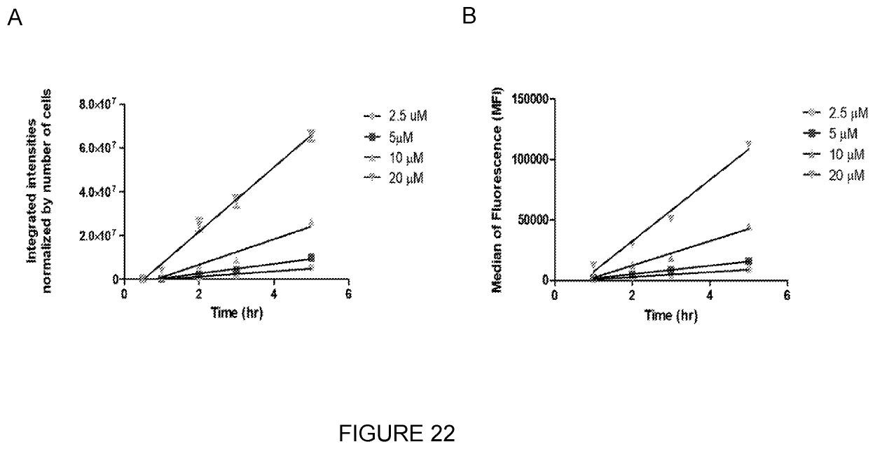

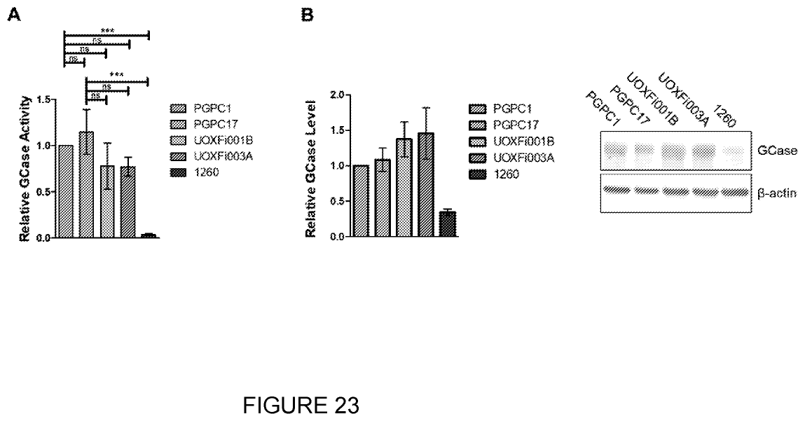

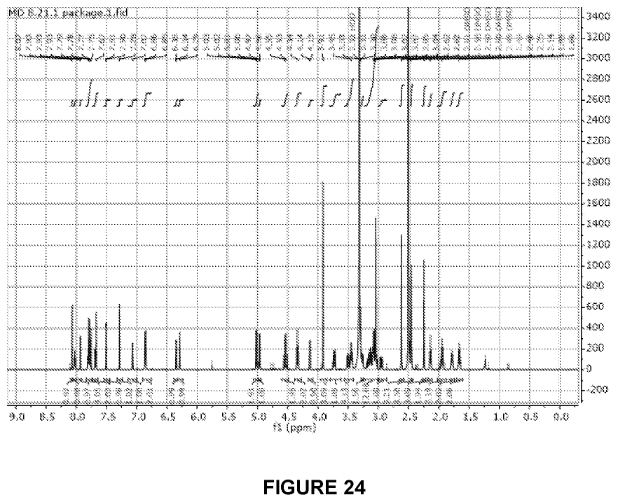

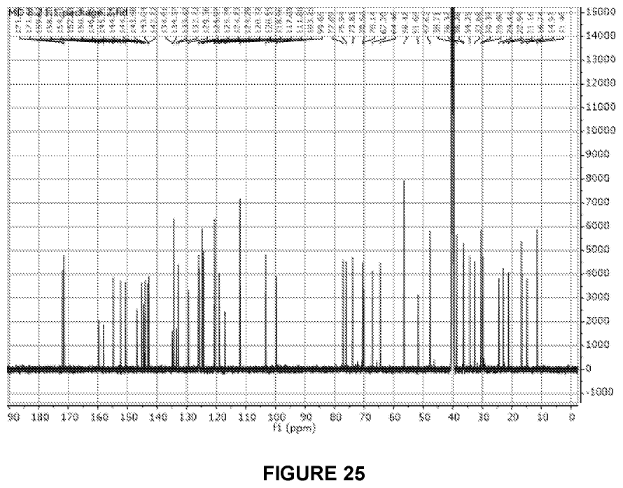

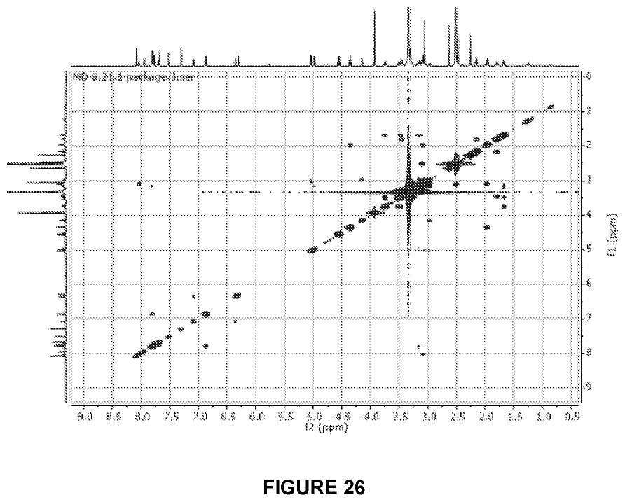

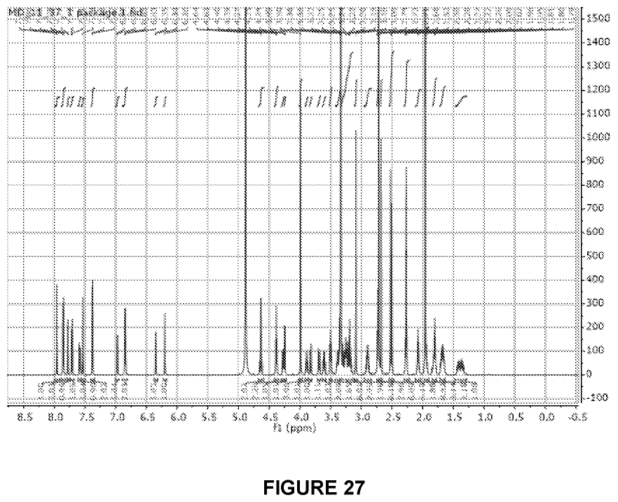

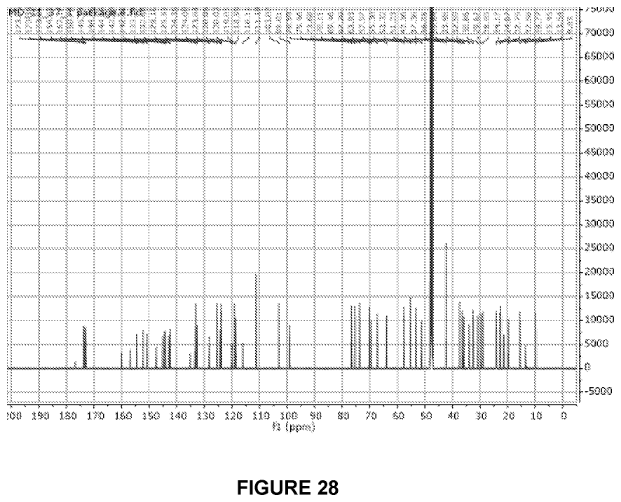

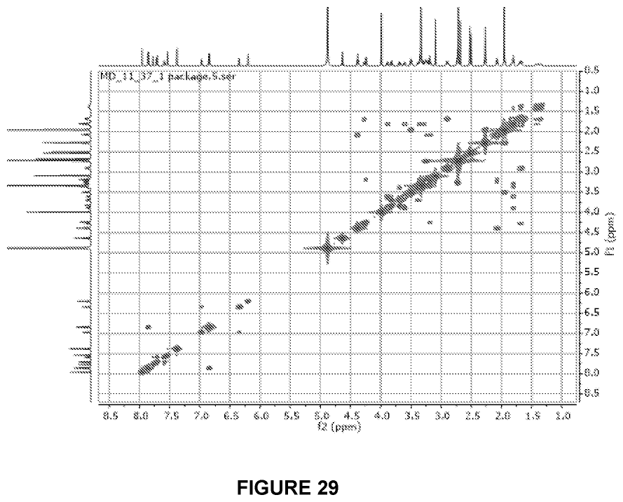

Substrate 4 was evaluated in fixed human neuronal cell culture model cells SK-N-SH. High content imaging of fixed SK-N-SH cells treated with DAPI, to stain nuclei, and 5 μM or 10 μM of substrate 4 or 9b revealed that the fixed signal from substrate 4 was far brighter than 9b. Cells plated in a 96-well clear-bottom Corning plate were treated with vehicle, 5 μM or 10 μM of substrate 4 or 9b in the presence or absence of 10 μM of the selective GCase inhibitor AT3375. After incubation at 37° C. and 5% CO₂ for 3 h, the media was removed, and the cells were washed. The cells were then fixed by treatment with paraformaldehyde in PBS (4%) for 30 minutes. The plate was then imaged using a Molecular Devices ImageXpress XLS High-Content Imager. The results indicated that the fluorescence quenched substrate 4 has improved signal intensity as compared to fluorescence quenched substrate 9b and after fixation by paraformaldehyde treatment (4% for 30 minutes) (FIG. 12). Additionally, while substrate 4 showed a doubling in fluorescent signal intensity with a doubling in substrate concentration there was no dose dependent difference in signal intensity observed when using 9b (FIG. 12). Cells treated with substrate 4 showed brighter signal and a clear doubling in signal when doubling substrate concentration which was not observed in the 9b treated cells (FIG. 12). Additionally, the signal observed was stable over the course of 6 days (FIGS. 15A-B), demonstrating that substrate 4 turnover is well fixed with in cells and indicating that it would be useful for quantitative measurements of GCase activity in fixed cells.

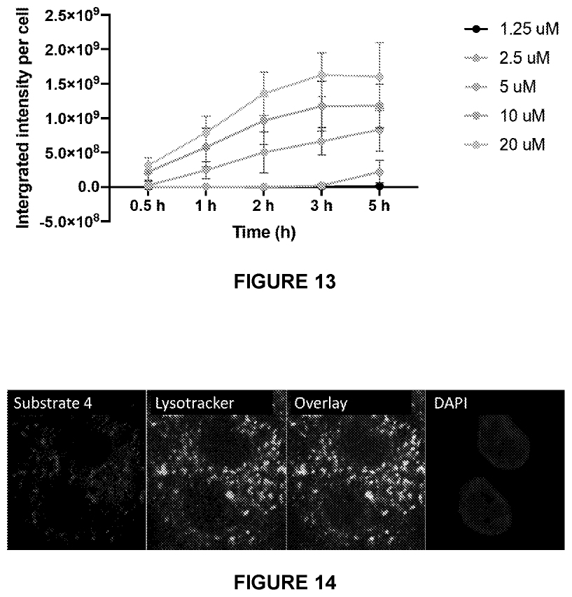

Using substrate 4, time dependent processing was established in fixed SK-N-SH cells. High content imaging of cells treated with DAPI, to stain nuclei, and different concentrations of substrate 4 revealed increasing fluorescence as a function of time. The fluorescence associated with cleavage of substrate 4 was present in a punctate pattern consistent with a lysosomal distribution. By integrating the fluorescence intensity from imaged fields, it was possible to observe a quantitative increase in fluorescence as a function of time (FIG. 13). The results indicated that fluorescence quenched substrate 4 shows time dependent increase in fixed SK-N-SH cells. From the images, the total mean integrated intensity was measured for the FITC channel and divided by the number of cells within the imaging site as determined by the DAPI staining. Measurements at various time points during incubation of various concentrations of substrate 4 reveals a linear increase from 30 minutes to 3 hours. Additionally, co-localization studies with immunocytochemistry staining of LAMP-2 positive vesicles further support that substrate 4 is dependent on lysosomal GCase activity (FIG. 14).

### Example 9

### Measurement of IC₅₀ Values of GCase Inhibitors in Fixed Cells

Substrate 4 can be used to measure the potency of a GCase inhibitor in fixed cells. High content imaging of SK-N-SH cells incubated for 3 h with DAPI, to stain nuclei, 10 μM of substrate 4 and varying concentrations of the selective GCase inhibitor AT3375 revealed decreasing fluorescence signal with increasing inhibitor concentration (FIG. 16). Cells were plated in a 96-well clear-bottom Corning plate and treated with vehicle, 10 μM of substrate 4 alone or 10 μM of substrate 4 with different concentrations of the GCase inhibitor AT3375 (triplicates). After incubation at 37° C. and 5% CO₂ for 2 h, the media was removed, and the cells were washed. The cells were then fixed by treatment with paraformaldehyde in PBS (4%) for 30 minutes. The plate was then imaged using a Molecular Devices ImageXpress XLS High-Content Imager. Integration of the mean fluorescence intensity per cell from randomly selected fields (9 sites per well), the percentage of activity (no inhibitor as a 100% activity control) was plotted against the log of the AT3375 inhibitor concentration to determine the IC₅₀ in cells. Importantly, the IC₅₀ value of AT3375 measured in fixed cells is in good agreement with the value measured using substrate 4 in live SK-N-SH cells (FIG. 16). These data reveal substrate 4 can be used to measure the effect of an inhibitor on GCase in fixed cells.

### REFERENCES

(1) Lombard, V.; Golaconda Ramulu, H.; Drula, E.; Coutinho, P. M.; Henrissat, B. The Carbohydrate-Active Enzymes Database (CAZy) in 2013. Nucleic Acids Res. 2014, 42 (D1), D490-D495.
(2) Bendikov-Bar, I.; Horowitz, M. Gaucher Disease Paradigm: From ERAD to Comorbidity. Hum. Mutat. 2012, 33 (10), 1398-1407.
(3) Cullen, V.; Sardi, S. P.; Ng, J.; Xu, Y.-H.; Sun, Y.; Tomlinson, J. J.; Kolodziej, P.; Kahn, I.; Saftig, P.; Woulfe, J.; et al. Acid β-Glucosidase Mutants Linked to Gaucher Disease, Parkinson Disease, and Lewy Body Dementia Alter α-Synuclein Processing. Ann. Neurol. 2011, 69 (6), 940-953.
(4) Migdalska-Richards, A.; Schapira, A. H. V. The Relationship between Glucocerebrosidase Mutations and Parkinson Disease. J. Neurochem. 2016, 139, 77-90.
(5) Grabowski, G. A. Phenotype, Diagnosis, and Treatment of Gaucher's Disease. Lancet 2008, 372 (9645), 1263-1271.
(6) Bennett, L. L.; Fellner, C. Pharmacotherapy of Gaucher Disease: Current and Future Options. P T 2018, 43 (5), 274-281.
(7) Platt, F. M.; Jeyakumar, M. Substrate Reduction Therapy. Acta Paediatr. 2008, 97 (s457), 88-93.
(8) Sawkar, A. R.; Cheng, W. C.; Beutler, E.; Wong, C. H.; Balch, W. E.; Kelly, J. W. Chemical Chaperones Increase the Cellular Activity of N370S β-Glucosidase: A Therapeutic Strategy for Gaucher Disease. Proc. Natl. Acad. Sci. U.S.A. 2002, 99 (24), 15428-15433.
(9) Richter, F.; Fleming, S. M.; Watson, M.; Lemesre, V.; Pellegrino, L.; Ranes, B.; Zhu, C.; Mortazavi, F.; Mulligan, C. K.; Sioshansi, P. C.; et al. A GCase Chaperone Improves Motor Function in a Mouse Model of Synucleinopathy. Neurotherapeutics 2014, 11 (4), 840-856.
(10) Fan, J. Q. A Contradictory Treatment for Lysosomal Storage Disorders: Inhibitors Enhance Mutant Enzyme Activity. Trends Pharmacol. Sci. 2003, 24 (7), 355-360.
(11) Aflaki, E.; Borger, D. K.; Moaven, N.; Stubblefield, B. K.; Rogers, S. A.; Patnaik, S.; Schoenen, F. J.; Westbroek, W.; Zheng, W.; Sullivan, P.; et al. A New Glucocerebrosidase Chaperone Reduces α-Synuclein and Glycolipid Levels in IPSC-Derived Dopaminergic Neurons from Patients with Gaucher Disease and Parkinsonism. J. Neurosci. 2016, 36 (28), 7441-7452.
(12) Neudorfer, O.; Giladi, N.; Elstein, D.; Abrahamov, A.; Turezkite, T.; Aghai, E.; Reches, A.; Bembi, B.; Zimran, A. Occurrence of Parkinson's Syndrome in Type 1 Gaucher Disease. QJM 1996, 89 (9), 691-694.
(13) Wong, K.; Sidransky, E.; Verma, A.; Mixon, T.; Sandberg, G. D.; Wakefield, L. K.; Morrison, A.; Lwin, A.; Colegial, C.; Allman, J. M.; et al. Neuropathology Provides Clues to the Pathophysiology of Gaucher Disease. Mol. Genet. Metab. 2004, 82 (3), 192-207.
(14) Tayebi, N. Gaucher Disease with Parkinsonian Manifestations: Does Glucocerebrosidase Deficiency Contribute to a Vulnerability to Parkinsonism? Mol. Genet. Metab. 2003, 79 (2), 104-109.
(15) Riboldi, G. M.; Di Fonzo, A. B. GBA, Gaucher Disease, and Parkinson's Disease: From Genetic to Clinic to New Therapeutic Approaches. Cells 2019, 8 (4), 364.
(16) Sidransky, E.; Lopez, G. The Link between the GBA Gene and Parkinsonism. Lancet Neurol. 2012, 11 (11), 986-998.
(17) Nalls, M. A.; Duran, R.; Lopez, G.; Kurzawa-Akanbi, M.; McKeith, I. G.; Chinnery, P. F.; Morris, C. M.; Theuns, J.; Crosiers, D.; Cras, P.; et al. A Multicenter Study of Glucocerebrosidase Mutations in Dementia with Lewy Bodies. JAMA Neurol. 2013, 70 (6), 727-735.
(18) Gegg, M. E.; Burke, D.; Heales, S. J. R.; Cooper, J. M.; Hardy, J.; Wood, N. W.; Schapira, A. H. V. Glucocerebrosidase Deficiency in Substantia Nigra of Parkinson Disease Brains. Ann. Neurol. 2012, 72 (3), 455-463.
(19) Winder-Rhodes, S. E.; Evans, J. R.; Ban, M.; Mason, S. L.; Williams-Gray, C. H.; Foltynie, T.; Duran, R.; Mencacci, N. E.; Sawcer, S. J.; Barker, R. A. Glucocerebrosidase Mutations Influence the Natural History of Parkinson's Disease in a Community-Based Incident Cohort. Brain 2013, 136 (2), 392-399.
(20) Brockmann, K.; Srulijes, K.; Pflederer, S.; Hauser, A. K.; Schulte, C.; Maetzler, W.; Gasser, T.; Berg, D. GBA-Associated Parkinson's Disease: Reduced Survival and More Rapid Progression in a Prospective Longitudinal Study. Mov. Disord. 2015, 30 (3), 407-411.
(21) Maple-Grodem, J.; Dalen, I.; Tysnes, O. B.; Macleod, A. D.; Forsgren, L.; Counsell, C. E.; Alves, G. Association of GBA Genotype With Motor and Functional Decline in Patients With Newly Diagnosed Parkinson Disease. Neurology 2021, 96 (7), e1036-e1044.
(22) Alcalay, R. N.; Levy, O. A.; Waters, C. C.; Fahn, S.; Ford, B.; Kuo, S. H.; Mazzoni, P.; Pauciulo, M. W.; Nichols, W. C.; Gan-Or, Z.; et al. Glucocerebrosidase Activity in Parkinson's Disease with and without GBA Mutations. Brain 2015, 138 (9), 2648-2658.
(23) Sardi, S. P.; Clarke, J.; Kinnecom, C.; Tamsett, T. J.; Li, L.; Stanek, L. M.; Passini, M. A.; Grabowski, G. A.; Schlossmacher, M. G.; Sidman, R. L.; et al. CNS Expression of Glucocerebrosidase Corrects α-Synuclein Pathology and Memory in a Mouse Model of Gaucher-Related Synucleinopathy. Proc. Natl. Acad. Sci. U.S.A. 2011, 108 (29), 12101-12106.
(24) Sardi, S. P.; Clarke, J.; Viel, C.; Chan, M.; Tamsett, T. J.; Treleaven, C. M.; Bu, J.; Sweet, L.; Passini, M. A.; Dodge, J. C.; et al. Augmenting CNS Glucocerebrosidase Activity as a Therapeutic Strategy for Parkinsonism and Other Gaucher-Related Synucleinopathies. Proc. Natl. Acad. Sci. U.S.A. 2013, 110 (9), 3537-3542.
(25) Kornhaber, G. J.; Tropak, M. B.; Maegawa, G. H.; Tuske, S. J.; Coales, S. J.; Mahuran, D. J.; Hamuro, Y. Isofagomine Induced Stabilization of Glucocerebrosidase. Chembiochem 2008, 9 (16), 2643-2649.
(26) Parmeggiani, C.; Catarzi, S.; Matassini, C.; D'Adamio, G.; Morrone, A.; Goti, A.; Paoli, P.; Cardona, F. Human Acid β-Glucosidase Inhibition by Carbohydrate Derived Iminosugars: Towards New Pharmacological Chaperones for Gaucher Disease. ChemBioChem 2015, 16 (14), 2054-2064.
(27) Goddard-Borger, E. D.; Tropak, M. B.; Yonekawa, S.; Tysoe, C.; Mahuran, D. J.; Withers, S. G. Rapid Assembly of a Library of Lipophilic Iminosugars via the Thiol-Ene Reaction Yields Promising Pharmacological Chaperones for the Treatment of Gaucher Disease. J. Med. Chem. 2012, 55 (6), 2737-2745.
(28) Dahl, M.; Doyle, A.; Olsson, K.; Minsson, J. E.; Marques, A. R. A.; Mirzaian, M.; Aerts, J. M.; Ehinger, M.; Rothe, M.; Modlich, U.; et al. Lentiviral Gene Therapy Using Cellular Promoters Cures Type 1 Gaucher Disease in Mice. Mol. Ther. 2015, 23 (5), 835-844.
(29) Van Patten, S. M.; Hughes, H.; Huff, M. R.; Piepenhagen, P. A.; Waire, J.; Qiu, H.; Ganesa, C.; Reczek, D.; Ward, P. V.; Kutzko, J. P.; et al. Effect of Mannose Chain Length on Targeting of Glucocerebrosidase for Enzyme Replacement Therapy of Gaucher Disease. Glycobiology 2007, 17 (5), 467-478.
(30) Witte, M. D.; Kallemeijn, W. W.; Aten, J.; Li, K. Y.; Strijland, A.; Donker-Koopman, W. E.; Van Den Nieuwendijk, A. M. C. H.; Bleijlevens, B.; Kramer, G.; Florea, B. I.; et al. Ultrasensitive in Situ Visualization of Active Glucocerebrosidase Molecules. Nat. Chem. Biol. 2010, 6 (12), 907-913.
(31) Ito, H.; Kawamata, Y.; Kamiya, M.; Tsuda-Sakurai, K.; Tanaka, S.; Ueno, T.; Komatsu, T.; Hanaoka, K.; Okabe, S.; Miura, M.; et al. Red-Shifted Fluorogenic Substrate for Detection of Lac Z-Positive Cells in Living Tissue with Single-Cell Resolution. Angew. Chemie 2018, 130 (48), 15928-15932.
(32) Asanuma, D.; Sakabe, M.; Kamiya, M.; Yamamoto, K.; Hiratake, J.; Ogawa, M.; Kosaka, N.; Choyke, P. L.; Nagano, T.; Kobayashi, H.; et al. Sensitive β-Galactosidase-Targeting Fluorescence Probe for Visualizing Small Peritoneal Metastatic Tumours in Vivo. Nat. Commun. 2015, 6, 6463.
(33) Ho, N. H.; Weissleder, R.; Tung, C. H. A Self-Immolative Reporter for β-Galactosidase Sensing. ChemBioChem 2007, 8 (5), 560-566.
(34) Komatsu, T.; Kikuchi, K.; Takakusa, H.; Hanaoka, K.; Ueno, T.; Kamiya, M.; Urano, Y.; Nagano, T. Design and Synthesis of an Enzyme Activity-Based Labeling Molecule with Fluorescence Spectral Change. J. Am. Chem. Soc. 2006, 128 (50), 15946-15947.
(35) Yadav, A. K.; Shen, D. L.; Shan, X.; He, X.; Kermode, A. R.; Vocadlo, D. J. Fluorescence-Quenched Substrates for Live Cell Imaging of Human Glucocerebrosidase Activity. J. Am. Chem. Soc. 2015, 137 (3), 1181-1189.
(36) Trapero, A.; Llebaria, A. Glucocerebrosidase Inhibitors for the Treatment of Gaucher Disease. Future Med. Chem. 2013, 5 (5), 573-590.
(37) Patnaik, S.; Zheng, W.; Choi, J. H.; Motabar, O.; Southall, N.; Westbroek, W.; Lea, W. A.; Velayati, A.; Goldin, E.; Sidransky, E.; et al. Discovery, Structure—Activity Relationship, and Biological Evaluation of Noninhibitory Small Molecule Chaperones of Glucocerebrosidase. J. Med. Chem. 2012, 55 (12), 5734-5748.
(38) Parenti, G. Treating Lysosomal Storage Diseases with Pharmacological Chaperones: From Concept to Clinics. EMBO Mol. Med. 2009, 1 (5), 268-279.
(39) Raghavan, S. S.; Topol, J.; Kolodny, E. H. Leukocyte β-Glucosidase in Homozygotes and Heterozygotes for Gaucher Disease. Am. J. Hum. Genet. 1980, 32 (2), 158-173.
(40) Deen, M. C.; Proceviat, C.; Shan, X.; Wu, L.; Shen, D. L.; Davies, G. J.; Vocadlo, D. J. Selective Fluorogenic β-Glucocerebrosidase Substrates for Convenient Analysis of Enzyme Activity in Cell and Tissue Homogenates. ACS Chem. Biol. 2020, 15 (4), 824-829.
(41) McKay, C. S.; Finn, M. G. Click Chemistry in Complex Mixtures: Bioorthogonal Bioconjugation. Chem. Biol. 2014, 21 (9), 1075-1101.
(42) Lang, K.; Chin, J. W. Bioorthogonal Reactions for Labeling Proteins. ACS Chem. Biol. 2014, 9 (1), 16-20.

All citations are hereby incorporated by reference.

The present invention has been described with regard to one or more embodiments. However, it will be apparent to persons skilled in the art that a number of variations and modifications can be made without departing from the scope of the invention as defined in the claims. Therefore, although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Elements listed with specific embodiments, are understood to be subject to combination in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

In the specification, the word “comprising” is used as an open-ended term, substantially equivalent to the phrase “including, but not limited to,” and the word “comprises” has a corresponding meaning. It is to be however understood that, where the words “comprising” or “comprises,” or a variation having the same root, are used herein, variation or modification to “consisting” or “consists,” which excludes any element, step, or ingredient not specified, or to “consisting essentially of” or “consists essentially of,” which limits to the specified materials or recited steps together with those that do not materially affect the basic and novel characteristics of the claimed invention, is also contemplated. Citation of references herein shall not be construed as an admission that such references are prior art to the present invention. All publications are incorporated herein by reference as if each individual publication was specifically and individually indicated to be incorporated by reference herein and as though fully set forth herein. The invention includes all embodiments and variations substantially as hereinbefore described and with reference to the examples and drawings.

1. A compound of Formula (I) or a salt thereof: 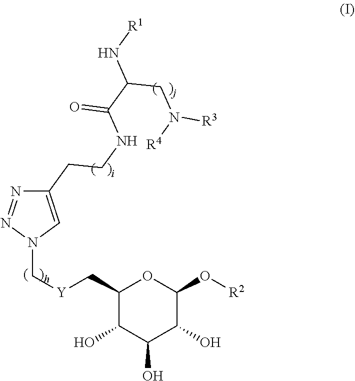
wherein
R¹ is a fluorophore or a quencher;
R² is 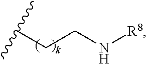
wherein k is an integer from 1 to 10, wherein when R¹ is a fluorophore, R⁸ is a quencher, or wherein when R¹ is a quencher, R⁸ is a fluorophore, or
R² is 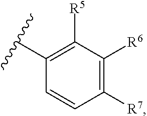
wherein R⁵, R⁶ and R⁷ each independently are H, F, NO₂, or a quencher, and R¹ is a fluorophore;
R³ and R⁴ are each independently H or C₁₋₄ alkyl;
h is an integer from 1 to 5;
i is an integer from 1 to 5;
j is an integer from 1 to 5;
k is an integer from 1 to 10; and
Y is O, S, NH or —CH₂.

2. The compound of claim 1 wherein:
R¹ is 4-((4-((E)-(2-methoxy-5-methyl-4-((E)-(4-methyl-2-nitrophenyl)diazenyl)phenyl)diazenyl)phenyl)(methyl)amino)butanoyl;
R⁸ is 3-(5,5-difluoro-7-(1H-pyrrol-2-yl)-5H-dipyrrolo[1,2-c:2l′-f][1,3,2]diazaborinin-4-ium-5-uid-3-yl)propanoyl;
h is 1;
i is 2;
k is 2; and
j is 3.

3. The compound of claim 1 wherein the compound is:
(S)-2-(3-(5,5-difluoro-7,9-dimethyl-dipyrrolo[1,2-c:2′,1′-f][1,3,2]diazaborinin-4-ium-5-uid-3-yl)propanamido)-6-(dimethylamino)-N-(3-(4-((((2R,3S,4S,5R,6R)-3,4,5-trihydroxy-6-(3-(4-((4-((E)-(2-methoxy-5-methyl-4-((E)-(4-methyl-2-nitrophenyl)diazenyl)phenyl)diazenyl)phenyl)(methyl)amino)butanamido)propoxy)tetrahydro-2H-pyran-2-yl)methoxy)methyl)-1H-1,2,3-triazol-1-yl)propyl)hexanamide 5-(((S)-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-6-(dimethylamino)-1-oxohexan-2-yl)carbamoyl)-2-(6-(dimethylamino)-3-(dimethyliminio)-3H-xanthen-9-yl)benzoate
5-(((S)-1-(((1-(4-(((2R,3S,4S,5R,6S)-6-(4-(2-(4-((4-((E)-(2,5-dimethoxy-4-((E)-(4-nitrophenyl)diazenyl)phenyl)diazenyl)phenyl)(methyl)amino)butanamido)ethyl)phenoxy)-3,4,5-trihydroxytetrahydro-2H-pyran-2-yl)methoxy)butyl)-1H-1,2,3-triazol-4-yl)methyl)amino)-6-(dimethylamino)-1-oxohexan-2-yl)carbamoyl)-2-(6-(dimethylamino)-3-(dimethyliminio)-3H-xanthen-9-yl)benzoate
5-(((2R)-6-(((((5aS,6R,6aR)-1-((S)-5-acetamido-6-(((S)-6-amino-1-(((S)-6-amino-1-(((S)-1,6-diamino-1-oxohexan-2-yl)amino)-1-oxohexan-2-yl)amino)-1-oxohexan-2-yl)amino)-6-oxohexyl)-1,4,5,5a,6,6a,7,8-octahydrocyclopropa[5,6]cycloocta[1,2-d][1,2,3]triazol-6-yl)methoxy)carbonyl)amino)-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate
5-(((2S)-6-(((((5aS,6R,6aR)-1-((S)-5-acetamido-6-(((S)-6-amino-1-(((S)-6-amino-1-(((S)-1,6-diamino-1-oxohexan-2-yl)amino)-1-oxohexan-2-yl)amino)-1-oxohexan-2-yl)amino)-6-oxohexyl)-1,4,5,5a,6,6a,7,8-octahydrocyclopropa[5,6]cycloocta[1,2-d][1,2,3]triazol-6-yl)methoxy)carbonyl)amino)-1-((3-(4-((((2R,3S,4S,5R,6R)-6-(3-(4-((4-((E)-(2,5-dimethoxy-4-((E)-(4-nitrophenyl)diazenyl)phenyl)diazenyl)phenyl)(methyl)amino)butanamido)propoxy)-3,4,5-trihydroxytetrahydro-2H-pyran-2-yl)methoxy)methyl)-1H-1,2,3-triazol-1-yl)propyl)amino)-1-oxohexan-2-yl)carbamoyl)-2-(6-(dimethylamino)-3-(dimethyliminio)-3H-xanthen-9-yl)benzoate
or an acceptable salt of any of the foregoing compounds.

4. The compound of claim 1 wherein the compound is a compound of Formula (II), or an acceptable salt thereof: 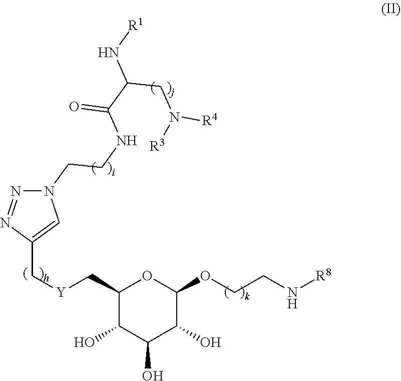
wherein
R¹ is a fluorophore and R⁸ is a quencher; or R¹ is a quencher and R⁸ is a fluorophore;
R³ and R⁴ each independently are hydrogen (H), C₁₋₄ alkyl, or a peptide of 1 to 10 amino acids in length that is covalently linked using a bioconjugation reaction or click reaction;
h is an integer from 1 to 5;
I is an integer from 1 to 5;
j is an integer from 1 to 5;
k is an integer from 1 to 10; and
Y is O or S or NH or CH₂; or
a compound of Formula (III), or an acceptable salt thereof: 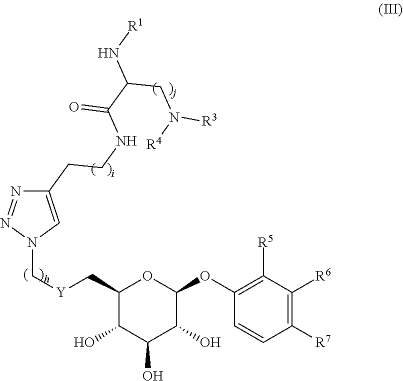
where R¹ is a suitable fluorophore;
R³ and R⁴ independently are H, C₁₋₄ alkyl, or a peptide of 1 to 10 amino acids in length that is covalently linked using a bioconjugation reaction or click reaction;
R⁵, R⁶ and R⁷ each independently are H, F, NO₂, or a suitable quencher;
h is an integer from 1 to 5;
i is an integer from 1 to 5;
j is an integer from 1 to 5; and
Y is O, S, NH or CH₂.

5. The compound of claim 4 wherein the compound of Formula (II) is:
i) a compound of Formula (IIa), or an acceptable salt thereof: 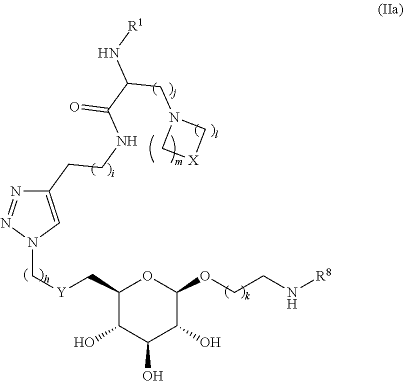
wherein
R¹ is a fluorophore and R⁸ is a quencher; or R¹ is a quencher and R⁸ is a fluorophore;
h is an integer from 1 to 5;
i is an integer from 1 to 5;
j is an integer from 1 to 5;
k is an integer from 1 to 10;
m is an integer from 1 to 4;
l is an integer from 1 to 4;
X is O, S, NH, CH₂ or NR⁹ wherein R⁹ is C₁₋₄ alkyl; and
Y is O, S, NH or CH₂;
ii) a compound of Formula (IIb), or an acceptable salt thereof: 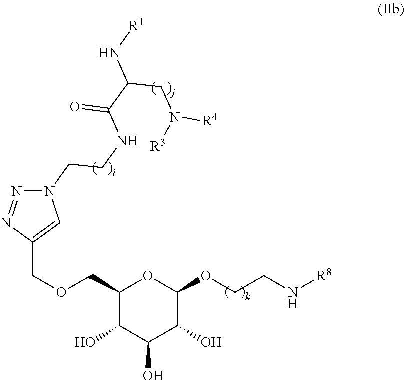
wherein
R¹ is a fluorophore and R⁸ is a quencher; or R¹ is a quencher and R⁸ is a fluorophore;
R³ and R⁴ each independently are H, C₁₋₄ alkyl, or a peptide of 1 to 10 amino acids in length that is covalently linked using a bioconjugation reaction or click reaction;
i is an integer from 1 to 5;
j is an integer from 1 to 5; and
k is an integer from 1 to 10;
iii) a compound of Formula (IIc), or an acceptable salt thereof. 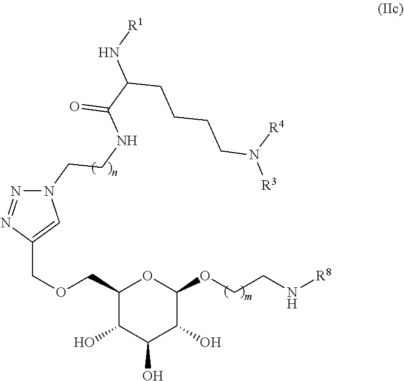
wherein
R¹ is a fluorophore and R⁸ is a quencher; or R¹ is a quencher and R² is a fluorophore;
R³ and R⁴ each independently are H, C₁₋₄ alkyl, or a peptide of 1 to 10 amino acids in length that is covalently linked using a bioconjugation reaction or click reaction;
n is an integer from 1 to 10; and
m is an integer from 1 to 5;
iv) a compound of Formula (IId), or an acceptable salt thereof. 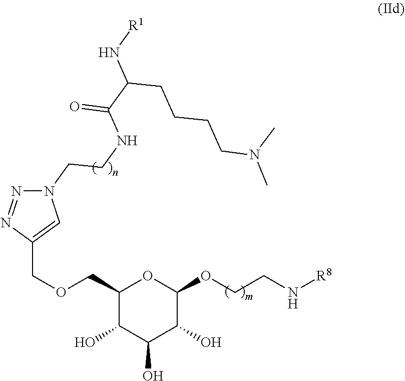
wherein
R¹ is a fluorophore and R⁸ is a quencher; or R¹ is a quencher and R⁸ is a fluorophore;
n is an integer from 1 to 10; and
m is an integer from 1 to 5;
v) a compound of Formula (IIe), or an acceptable salt thereof. 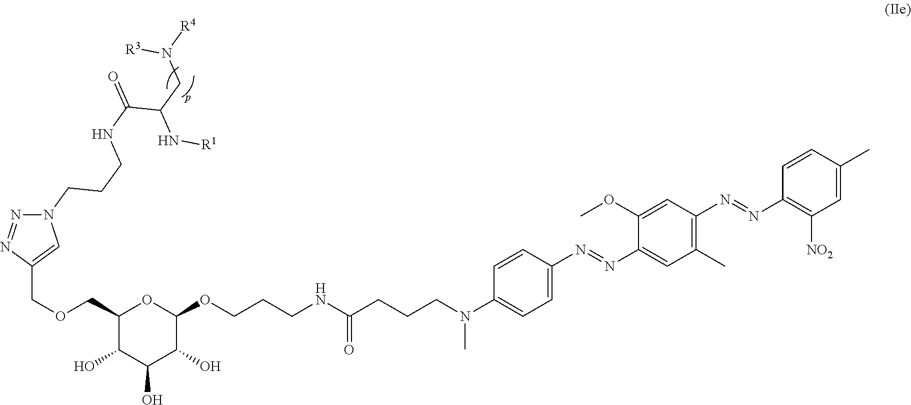
where
R¹ is a suitable fluorophore;
R³ and R⁴ each independently are H, C₁₋₄ alkyl, or a peptide of 1 to 10 amino acids in length that is covalently linked using a bioconjugation reaction or click reaction;
n is an integer from 1 to 10;
m is an integer from 1 to 5; and
p is an integer from 1 to 5;
vi) a compound of Formula (IIf), or an acceptable salt thereof. 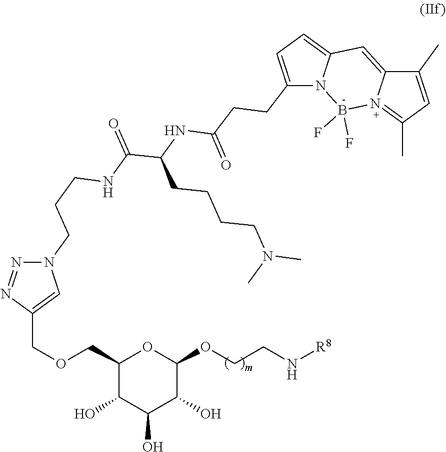
where R⁸ is a suitable quencher; and
m is an integer from 1 to 5;
vii) a compound of Formula (IIg), or an acceptable salt thereof: 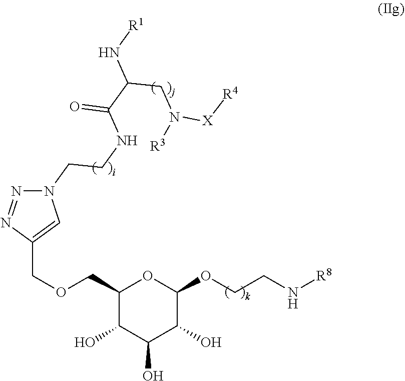
wherein
R¹ is a fluorophore and R⁸ is a quencher; or R¹ is a quencher and R⁸ is a fluorophore;
R³ is H or C₁₋₄ alkyl;
R⁴ is a peptide between 1 to 10 amino acid residues in length;
X is the product of a click reaction;
i is an integer from 1 to 5;
j is an integer from 1 to 5; and
k is an integer from 1 to 10;
viii) a compound of Formula (IIh), or an acceptable salt thereof: 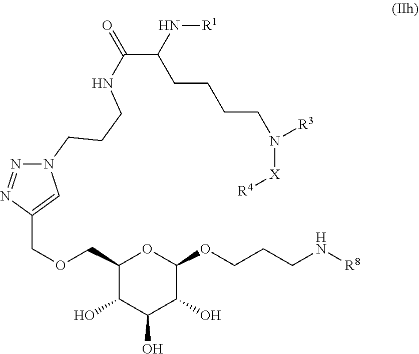
wherein
R¹ is a fluorophore and R⁸ is a quencher; or R¹ is a quencher and R⁸ is a fluorophore;
R³ is H or C₁₋₄ alkyl;
R⁴ is a peptide between 1 to 10 amino acid residues in length; and
X is the product of a click reaction;
ix) a compound of Formula (IIi), or an acceptable salt thereof: 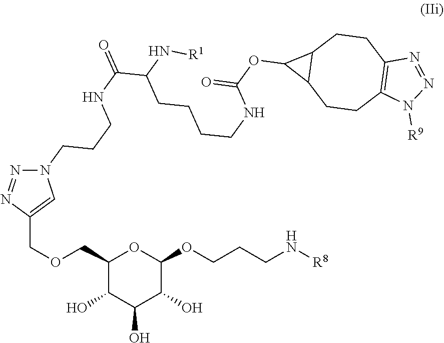
wherein
R¹ is a fluorophore and R⁸ is a quencher; or R¹ is a quencher and R⁸ is a fluorophore; and
R⁹ is a peptide between 1 to 10 amino acid residues in length;
x) a compound of Formula (IIj), or an acceptable salt thereof: 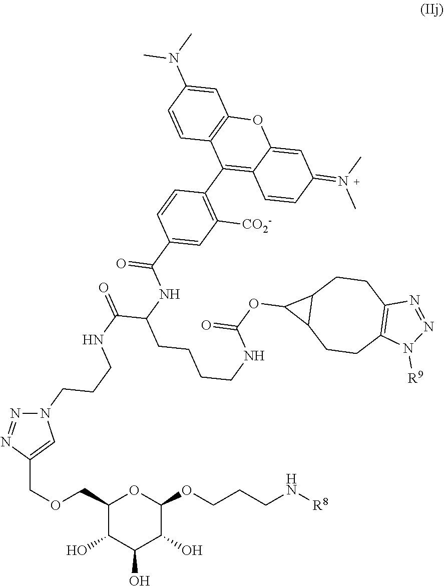
wherein
R² is a quencher; and
R⁹ is a peptide between 1 to 10 amino acids in length.
xi) a compound of Formula (IIk), or an acceptable salt thereof: 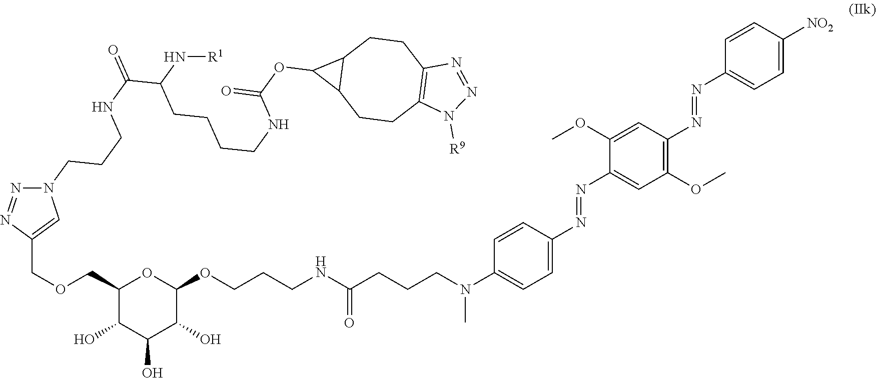
wherein
R¹ is a fluorophore; and
R⁹ is a peptide between 1 to 10 amino acid residues in length; or
xii) a compound of Formula (IIl), or an acceptable salt thereof: 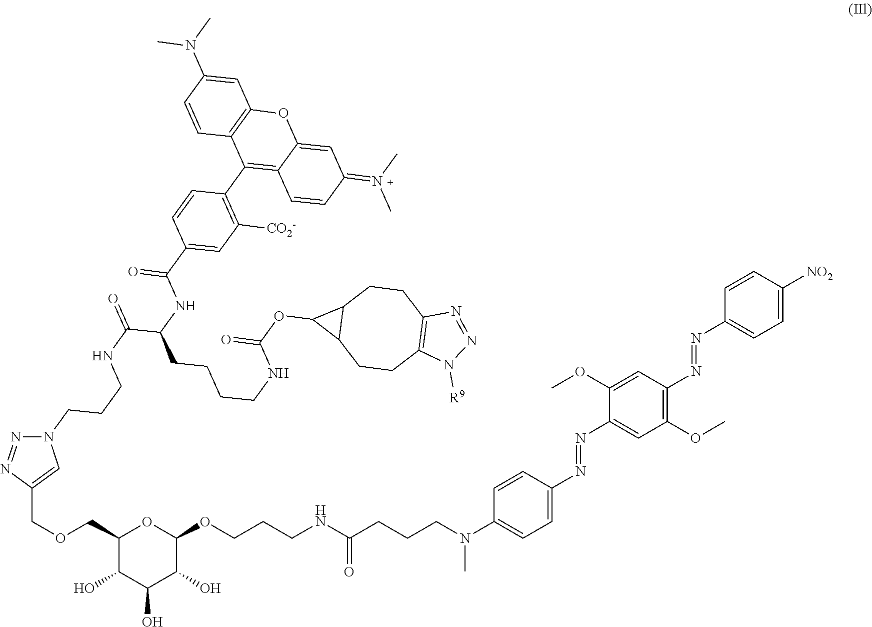
wherein R⁹ is a peptide between 1 to 10 amino acid residues in length.

6. The compound of claim 4 wherein the compound of Formula (III) is:
i) a compound of Formula (IIIa), or an acceptable salt thereof: 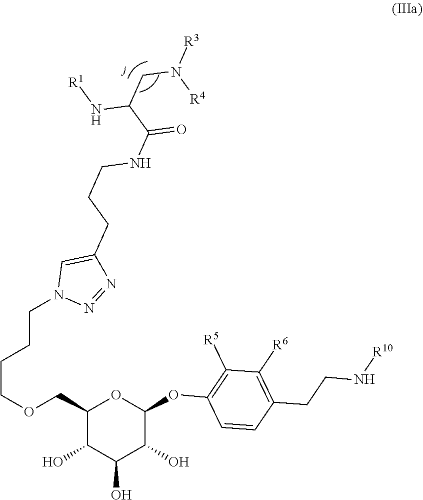
wherein
R¹ is a fluorophore;
R³ and R⁴ each independently are H, C₁₋₄ alkyl or a peptide of 1 to 10 amino acids in length that is covalently linked using a bioconjugation reaction or click reaction;
R⁵ and R⁶ each independently are H, F, or NO₂;
R¹⁰ is a quencher; and
j is an integer from 1 to 5;
ii) a compound of Formula (IIIb), or an acceptable salt thereof: 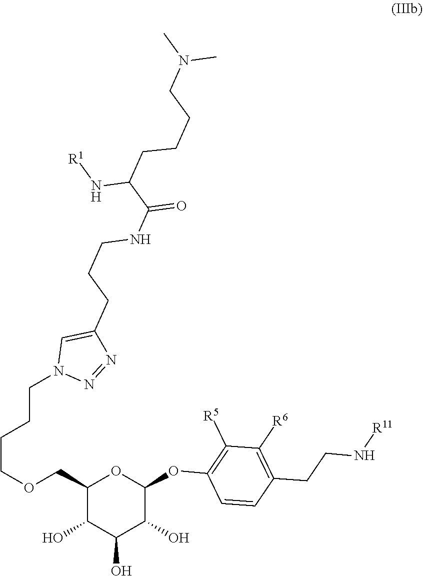
wherein
R¹ is a fluorophore;
R⁵ and R⁶ each independently are H, F, or NO₂; and
R¹¹ is a quencher;
iii) a compound of Formula (IIIc), or an acceptable salt thereof: 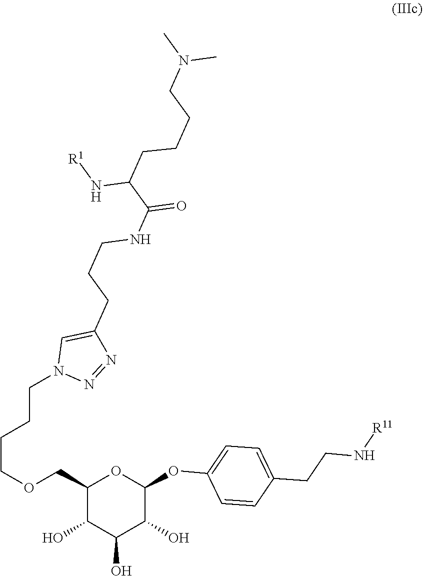
wherein
R¹ is a fluorophore; and
R¹¹ is a quencher;
iv) a compound of Formula (IIId), or an acceptable salt thereof: 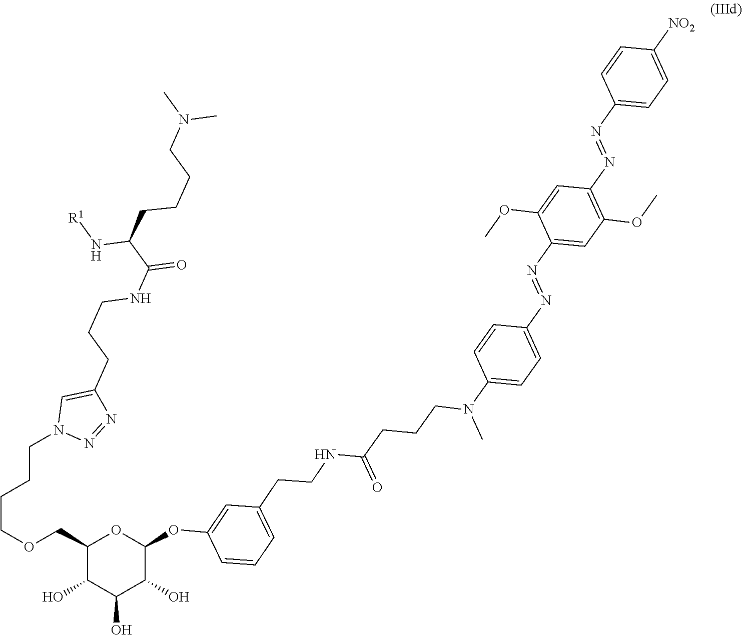
wherein R¹ is a fluorophore; or
v) a compound of Formula (IIIe), or an acceptable salt thereof: 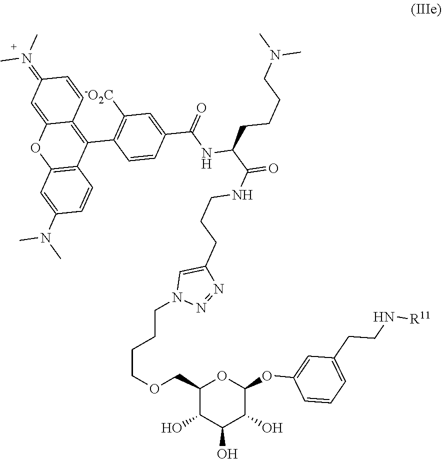
wherein R¹¹ is a quencher.

7. The compound of claim 1 wherein the compound is a fluorescence-quenched substrate for an acid β-glucosidase (GCase).

8. The compound of claim 7 wherein the GCase is a mammalian GCase.

9. A method for determining or localizing GCase activity in a cell, the method comprising:
a) providing a test cell and a control cell;
b) contacting the test cell with the compound of claim 1;
c) determining fluorescence intensity in the test cell and the control cell, wherein an increase in fluorescence intensity of the test cell when compared to the control cell is indicative of GCase activity, or visualizing fluorescence intensity in the test cell and the control cell, wherein an increase in fluorescence intensity in a location in the test cell when compared to the fluorescence intensity in a corresponding location in the control cell is indicative of GCase activity.

10. (canceled)

11. The method of claim 9 wherein the location is the endoplasmic reticulicum, Golgi apparatus or lysosomal compartment.

12. The method of claim 9 further comprising determining the effect of a GCase modulator in a cell, the method comprising:
contacting the test cell with a GCase modulator;
wherein a difference in fluorescence intensity of the test cell when compared to the control cell is indicative of GCase modulation.

13. The method of claim 12 wherein the GCase modulator is a GCase inhibitor or a GCase activator or a GCase chaperone.

14. A method for determining the efficacy of a GCase-directed therapy, the method comprising:
a) providing a test cell, wherein the test cell is obtained from a subject treated with a GCase-directed therapy, and a control cell, wherein the control cell is obtained from a subject not treated with a GCase-directed therapy;
b) contacting the test cell and the control cell with the compound of claim 1;
c) measuring fluorescence intensity in the test cell and the control cell, wherein a difference in fluorescence intensity of the test cell when compared to the control cell is representative of the efficacy of the GCase-directed therapy.

15. The method of claim 9 wherein the cells are fibroblasts or PBMCs.

16. The method of claim 9 wherein the cells are derived from a tissue.

17. The method of claim 16 wherein the tissue is a skin punch.

18. A method of screening for a GCase activity enhancer, the method comprising:
a) providing a test cell and a control cell;
b) contacting the test cell with a test compound;
c) contacting the test cell and the control cell with the compound of claim 1;
d) determining fluorescence intensity for the test cell and the control cell, wherein an increase in fluorescence intensity in the test cell when compared to the control cell indicates that the test compound is a GCase activity enhancer.

19. The method of claim 9 wherein:
R¹ is 4-((4-((E)-(2-methoxy-5-methyl-4-((E)-(4-methyl-2-nitrophenyl)diazenyl)phenyl)diazenyl)phenyl)(methyl)amino)butanoyl;
R⁸ is 3-(5,5-difluoro-7-(1H-pyrrol-2-yl)-5H-dipyrrolo[1,2-c:2\l′-f][1,3,2]diazaborinin-4-ium-5-uid-3-yl)propanoyl;
h is 1;
i is 2;
k is 2; and
j is 3.

20. A compound of claim 1 or a salt thereof wherein:
k is 2; and
j is 3.


SEQUENCE LISTING (5 sequences)

SEQ ID NO: 1 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - protein, 41 aa, Homo sapiens
QSVRLGSWDRGMQYSHSIITSLLYHVVGWTDWNLALNPEGG

SEQ ID NO: 3 - protein, 41 aa, Homo sapiens
SKFIPEGSQRVGLVASQKNDPDAVALMHPDGSAVVVVLNRS

SEQ ID NO: 4 - protein, 41 aa, Homo sapiens
GKGSLKGQPGDIYHQTWARYIVKFLDAYAEHKLQFWAVTAE

SEQ ID NO: 5 - protein, 41 aa, Homo sapiens
PTWLKTNGAVNGKGSLKGQPRDIYHQTWARYFVKFLDAYAE